(19) United States
(12) Patent Application Publication
(10) Pub. No.: US 2025/0186609 A1
(43) Pub. Date: Jun. 12, 2025

(54) SIALIC-ACID LIGAND DECORATED THERAPEUTICS

(71) Applicant: AVICEDA OPHTHALMICS, INC., Cambridge, MA (US)

(72) Inventors: Michael Tolentino, Lakeland, FL (US); Derek Y Kunimoto, Paradise Valley, AZ (US); Anitha Krishnan, Braintree, MA (US); Mohamed A. Genead, Newton Center, MA (US); Rajesh Shinde, Lexington, MA (US); Gerardus J.P.H. Boons, Athens, GA (US); Quiang Liu, Zhongshan City (CN); Anthony R. Prudden, Athens, GA (US); Pradeep Chopra, Athens, GA (US)

(21) Appl. No.: 18/026,535

(22) PCT Filed: Sep. 16, 2021
(86) PCT No.: PCT/US21/50671

§ 371 Date: Mar. 15, 2023

Related U.S. Application Data
(60) Provisional application No. 63/079,292, filed on Sep. 16, 2020.

(51) Int. Cl. A61K 47/69; A61K 9/00; A61K 9/19; A61K 47/59; A61K 47/60; A61P 27/02

(52) U.S. Cl. CPC A61K 47/6937; A61K 9/0048; A61K 9/19; A61K 47/593; A61K 47/60; A61P 27/02

(57) ABSTRACT

The present disclosure provides methods and compositions for modulating the activity of self-associated molecular pattern recognition receptors such as for example, Siglec (sialic-acid-binding immunoglobulin-type lectins) and complement factor H (CFH). Modulating the activity of infectious organisms such as viral influenza A, B, C, SARS-CoV1, 2, and cancer/tumor cells such as lung, breast and skin cancers. The compositions comprise a particle, comprising a molecule represented by the following structural formula:

P-L-G,

wherein P is a biocompatible polymer scaffold comprising at least one biocompatible polymer defined herein, G is a polysialic acid (PSA) comprising from 5 to 200 repeat units of sialic acid; and L is a covalent linker, or a pharmaceutically acceptable salt thereof.

This application is the U.S. National Stage of International Application No. PCT/US2021/050671, filed Sep. 16, 2021, which designates the U.S., published in English, and claims the benefit of U.S. Provisional Application Nos. 63/079.292, filed on Sep. 16, 2020. The entire teachings of the above applications are incorporated herein by reference.

### TECHNICAL FIELD

The present disclosure provides methods and compositions for modulating the activity of self-associated pattern recognition receptors such as, for example, Siglecs (sialic-acid-binding immunoglobulin-type lectins) and complement factor H (CFH). The provided compositions include, for example, nanoparticles, microparticles, other polymer structures decorated with glycan structures that have end terminal sialic-acids that bind to, agonize or antagonize, self-associated molecular pattern recognition receptors and infectious associated sialic-acid binding moieties that allow entry, propagation and evasion of immune surveillance in the host.

The binding to such self-associated pattern recognition receptors, and/or agonizing or antagonizing their activity, can resolve innate, adaptive, multimodal, inflammatory, or complement-mediated immune responses, thereby providing treatment of diseases of: (1) acute inflammation such as viral, bacterial, allergen, transplant rejection, or autoimmune induced inflammation: (2) chronic inflammation such as chronic obstructive pulmonary disease, or rheumatic disorders; and (3) chronic non-resolving inflammation of the innate and adaptive form such as exudative or non-exudative macular degeneration, or Alzheimer's disease.

The provided compositions can also be used to block, or antagonize, self-associated molecular pattern recognition receptors, which allow cancer cells, infectious agents such as viral, bacterial, helminthic, parasitic or damaged-associated molecular patterns (DAMP) to evade immune surveillance, detection and clearance by the innate or adaptive immune system.

The provided composition can also be used to prevent infectious agents such as Haemophilus influenzae, SARS-CoV-1, SARS-CoV-2, and Streptococcus from entering host cells by binding to the sialic-acid receptor and/or sialidase that attaches to host's sialic-acid and facilitates entry and propagation in the host cell.

### BACKGROUND

The ability to recognize self is what down regulates the body's immune system so that it does not destroy its own healthy host cells. The composition of the glycome (carbohydrate moieties that coat all cells) of a particular cell determines whether a cell is recognized as a self-associated cell, non-self-cell, or damaged cell. During the process of immune surveillance and inflammation, the immune system checks the glycome signature of an encountered cell to determine if the cell requires elimination via immune activation, or if the cell constitutes an undamaged host cell that should signal a suppression of immune activation or inflammatory resolution. The receptors or binding regions found on inflammatory cells that are responsible for recognizing this glycome signature are considered self-associated pattern recognition receptors.

The largest family of self-associated molecular pattern recognition receptors are called Siglecs (sialic-acid-binding immunoglobulin-type lectins). Currently there are 16 described Siglecs. Siglecs are found on the surface of inflammatory cells with different Siglec expression patterns found on different inflammatory cells. When presented with a specific sialic-acid ligand pattern on the surface of a healthy host cell, an agonized inhibitory Siglec receptor will activate the immunoglobulin tyrosine kinase inhibitory motif (ITIM), which recruits src homology 2 domain-containing protein tyrosine phosphatase 1 and 2 (SHP-1 and 2), both phosphatases that dephosphorylate kinases that keep the inflammatory cell in an activated state. This inhibitory Siglec-controlled mechanism can shut down activated inflammatory cells profoundly, resulting in resolution of inflammation. Different Siglecs have different sialic-acid signatures that bind and agonize the receptor, resulting in the profound deactivation of inflammatory cells.

Agonizing Siglec 3, 5, 7, 9, 10, 11, or 15 will dephosphorylate all the activated (phosphorylated) tyrosine kinases within a given cell resulting in intracellular shut down of activation of that particular cell. The sialic-acid ligands as well as the density and presentation of these ligands to the particular Siglec receptor determine its ability to agonize, antagonize, or block the receptor binding site.

Antagonizing Siglec 14 or 16, which activate inflammation via the immunoglobulin tyrosine kinase activation motif (ITAM) is another mechanism to deactivate inflammation. When Siglec 14 or 16 are agonized, ITAM is activated and the tyrosine residues within ITAM become phosphorylated by the SRC family of kinases, which creates a conformational change allowing the motif to become a docking site for proteins containing the SH2 domain. Agonizing Siglec 14 and 16 also can be used to activate inflammation for the treatment of infectious diseases or in the field of oncology.

Complement factor H (CFH) represents another sialic-acid binding self-associated pattern recognition receptor. CFH is responsible for resolving activation of the complement cascade, especially the alternative complement pathway. CFH downregulates the complement cascade by binding and degrading complement factor 3Bb (C3Bb). C3Bb represent the amplification factor that propagates the complement cascade. CFH is the central regulator of the alternative complement pathway. Because the alternative complement pathway is constitutively activated CFH acts as the brakes that prevent the complement cascade from accelerating and causing cellular lysis through the membrane attack complex. When CFH is activated and binds with an appropriate self-associated molecular pattern (a sialic acid ligand presented to CFH appropriately) then CFH binds to C3Bb which prevents Bb from binding C3Bb preventing the formation of C3BbBb. C3BbBb is the rate limiting intermediate that propagates and accelerates the complement cascade. If CFH is not bound to an appropriate self-associated molecular pattern (sialic acid ligand presented in the correct configuration) then CFH does not bind to C3Bb resulting in the accelerated formation of C3BbBb and amplified formation of membrane attack complex and unbridled cell lysis of both pathogenic and native cells.

CFH is made up of 20 complement control protein (CCP) modules. There are two sialic-acid binding regions on CFH, the 4-6 CCP region and the 19-20 CCP region of CFH. Binding of these regions by sialic acid is believed to open the binding area for C3Bb, which would deactivate the complement cascade. It is believed that simultaneous binding of these 2 sites is required to form a conformational change in CFH that enhances its binding to C3Bb.

Antagonizing or blocking the binding site of Siglec 3, 5, 7, 9, 10, 11, or 15 is a method for treating conditions that agonize Siglec with self-associated molecular pattern (SAMP)-mimicking surface sialic-acid ligands to evade immune surveillance or immune activation. Conditions that use this method include cancer and infections. Cancers have been shown to express sialic-acid structures on their surface to evade immune activation of macrophages, natural killer (NK) cells, and monocytes. Streptococcus B also expresses a sialic-acid ligand on its surface that binds Siglec 7 to avoid immune attack.

Sialic-acid is also used as an entry point for several family of viruses such as Influenza A. Influenza B, Influenza C, SARS-CoV-1, or SARS-CoV-2. Binding receptors on these viruses can be hemagglutinin esterase (viral HE), Neuraminidase (viral N), or a viral capsid moiety such as spike protein (viral SP) that binds to sialic-acid ligands on the surface of host cells and facilitates viral entry into host cells, or CD147 which is a sialic-acid binding lectin used for infective entry by SARS-CoV-2 and Plasmodium falciparum. Binding these sialic-acid receptors with a decoy sialic-acid ligand can prevent virus from infecting host cells as well as prevent egress of viral particles from an infected cell.

A need exists for improved compositions and methods for agonizing sialic-acid binding self-associated pattern recognition receptors, for treatment of diseases resulting from acute, chronic, or aberrant immune system activation. A need also exists for blocking these SAMP receptors from being commandeered by cancer and infections to avoid immune surveillance and attack. The present invention provides for nanoparticles that can present sialic-acid ligands in a density that will agonize, block, or antagonize a particular Siglec receptors specifically and profoundly.

### SUMMARY

In one embodiment, the present invention is a particle, comprising a molecule represented by the following structural formula:

P-L-G,

wherein: P is a biocompatible polymer scaffold comprising at least one biocompatible polymer selected from the group consisting of polyglycolic acid, poly(L-lactic acid), poly(lactic-co-glycolic acid), polycaprolactone, poly(3-hydroxybutyric acid), poly(ethylene glycol), polyethylene oxide, Pluronic F127, Pluronic F68, poloxamer, poly(hydroxymethylmethacrylate), polyvinyl alcohol, poly(vinvlpyrrolidone), hyaluronic acid, heparin, heparin sulfate, sialic-acid and chitosan; G is a polysialic acid (PSA) comprising from 5 to 200 repeat units of sialic acid; and L is a covalent linker, or a pharmaceutically acceptable salt thereof.

In another embodiment, the present invention is a method of treating a subject suffering from an ophthalmic disease, comprising: administering to the subject a therapeutically effective amount of a particle according to any of the embodiments described herein.

In another embodiment, the present invention is a method of treating a subject suffering from cancer, comprising: administering to the subject a therapeutically effective amount of a particle according to any of the embodiments described herein, wherein the cancer is acute lymphoblastic leukemia, chronic myelogenous leukemia, acute myelogenous leukemia, non-Hodgkin's or Hodgkin's lymphoma.

In another embodiment, the present invention is a method of treating a subject suffering from an infectious disease, comprising: administering to the subject a therapeutically effective amount of a particle according to any of the embodiments described herein, wherein the infectious disease is caused by Streptococcus group B, Streptococcus pneumonia, E. coli, Pseudomonas aeruginosa, Neisseria meningitidis, Campylobacter jejuni, Tyrpanosoma cruzi, HIV, influenza A, B, or C, Sars CoV1, Sars Co V2, or Herpes viridae.

In another embodiment, the present invention is method of modulating a cell-mediated inflammatory response in an immune cell, comprising: contacting the immune cell with a particle according to any of the embodiment described herein.

In another embodiment, the present invention is a pharmaceutical composition comprising a particle according to any of the embodiment described herein, and a pharmaceutically acceptable carrier.

In another embodiment, the present invention is a method of inhibiting complement activation in a subject, the method comprising: administering to the subject a therapeutically effective amount of a particle according to any of the embodiments described herein.

In another embodiment, the present invention is a method of treating a subject suffering from complement hyperactivation disease, comprising administering to the subject a therapeutically effective amount of a particle according to any of the embodiments described herein.

In another embodiment, the present invention is a method of manufacturing a particle, the method comprising: reacting a biocompatible polymer scaffold P, the biocompatible polymer scaffold P comprising a first labile moiety, and a glycan G, the glycan G comprising a second labile moiety, under the condition sufficient to produce an adduct L of the first labile moiety and a second labile moiety, thereby producing a molecule represented by the following structural formula:

P-L-G,

wherein: P comprises at least one biocompatible polymer selected from the group consisting of polyglycolic acid, poly(L-lactic acid), poly(lactic-co-glycolic acid), polycaprolactone, poly(3-hydroxybutyric acid), poly(ethylene glycol), polyethylene oxide, Pluronic F127, Pluronic F68, poloxamer, poly(hydroxymethylmethacrylate), polyvinyl alcohol, poly(vinylpyrrolidone), hyaluronic acid, heparin, heparin sulfate, sialic-acid and chitosan; and G is a polysialic acid (PSA) comprising from 5 to 200 repeat units of sialic acid.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments disclosed herein.

FIG. 1 Modular nanoparticle with dibenzocyclooctyne (DBCO) functional group for copper (CU)-free click chemistry.

FIG. 2. Modular nanoparticle with alkyne functional group for catalyzed alkyne-azide cycloaddition (CuAAC) click chemistry. The copper-free Click Chemistry is based on the reaction of an alkyne such as a dibenzylcyclooctyne (DBCO) moiety with an azide-labeled moiety. This reaction is known as strain-promoted alkyne azide cycloaddition (SPAAC).

FIG. 3. Polysialic-acid azide ligand synthesis.

FIG. 4. Modular nanoparticle using DBCO for conjugation of sialyllactose-polysialic azide-azide ligand.

FIG. 5. 2-3 sialyllactose or 2-6 sialyllactose ligand conjugation to modular PLGA-PEG-DBCO nanoparticle.

FIG. 6. Reaction of alkyne-PEG-PLGA nanoparticles with polysialic-acid-azide using CuAAC click chemistry to form polysialic-acid-functionalized nanoparticles.

FIG. 7. Reaction of alkyne-PEG-PLGA nanoparticles with NeuAcα2-3Galβ1-4Glc-azide using CuA AC click chemistry to form polysialic-acid-functionalized nanoparticles.

FIG. 8. Reaction of nanoparticles formed with alkyne-PEG-PLGA and tetrazine-PEG-PLGA with polysialic acid-azide and NeuAcα2-3Galβ1-4Glc-trans-cyclooctene respectively using CuAAC click chemistry to form nanoparticles functionalized with polysialic acid and NeuAcα2-3Galβ1-4Glc.

FIG. 9. Cellular mechanism for modulation of Siglec activity.

FIG. 10. Nanoparticles illustrating different densities of ligands.

FIG. 11. Sialic acid nanoparticles bind with high affinity and in a dose dependent fashion (a) to siglec 11 (b) to siglec 9 (c) to siglec 7 (d) to siglec 5.

FIG. 12. Sialic acid nanoparticles are non-toxic to (a) peripheral blood monocyes and (b) THP-1 monocyte derived macrophages as demonstrated by an MTT assay.

FIG. 13. Showing that exemplary nanoparticles increase IL-10 in activated macrophages.

FIG. 14A and FIG. 14B, collectively are bar plot showing that exemplary nanoparticles increase CFH production.

FIG. 15 Showing that exemplary nanoparticles inhibit TNF-alpha in LPS-activated macrophages.

FIG. 16 Showing that exemplary nanoparticles significantly reduce VEGF in LPS-activated macrophages.

FIG. 17 is a synthetic scheme for a non-reducing end conjugation of PSA to an azide functional group.

FIG. 18 is a schematic diagram of a work flow for Thermal Hydrolysis of PSA.

FIG. 19 is a by ¹H-NMR spectrum of hydrolyzed PSA used to determine its Degree of Polymerization (DP).

FIG. 20 is a bar plot showing suppression of TNF-alpha production in LPS-challenged THP-1 cells following incubation with nanoparticles described herein.

FIG. 21 is a bar plot showing dose dependent increase in anti-inflammatory mediator IL-10 in LPS-challenged M1 macrophages following treatment with AT-007-NP04.

FIG. 22A and FIG. 22B show down-regulation of proinflammatory mediators IL-6 (B) and TNF-α (A) in LPS-challenged M1 macrophages following treatment with AT-007-NP06.

FIG. 23 is a bar plot showing the outer nuclear layer thickness (μM) from animals injected with blank-NP. AT-007 and AT-007-NP02.

FIG. 24 is a schematic diagram of the experimental setup used to measure the effect of AT-007-NP04 on differentiation of fibrocytes.

FIG. 25 is a bar plot showing % fibrocyte differentiation following treatment with the indicated agent.

FIG. 26 is a schematic diagram of the experimental setup used to measure the effect of AT-007-NP06 in delaying NET formation and delaying cell death measured using Sytox orange dye.

FIG. 27 shows the kinetic curve as a function of time for the % SyTOX positive.

FIG. 28 is a schematic diagram of Complement Pathway.

FIG. 29 is a schematic diagram of the nanoparticles described herein binding to C3b Displacing Bb to shut down complement amplification.

FIG. 30A is a sensogram illustrating a strong binding of AT-007-NP04 on complement factor H enhanced binding to C3b determined by surface plasmon resonance (SPR-Biacore).

FIG. 30B is a sensogram illustrating a strong binding of AT-007-NP04 on complement factor H enhanced binding to C3b determined by surface plasmon resonance (SPR-Biacore).

FIG. 31A is a schematic diagram illustrating the setup of an experiment results of which are shown in FIG. 31B and described herein.

FIG. 31B is a bar plot illustrating the ability of AT-007-NP06 nanoparticle to directly prevent complement activation via alternative or classical pathway.

FIG. 32A, FIG. 32B, and FIG. 32C are the plots showing the ELISA-measured binding affinity of AT-007-NP04 and AT-007-NP06 to CFH protein.

FIG. 33 is a plot demonstrating that batch AT-007-NP04 exhibits binding affinity towards Siglec 7, 9 and 11 as measured by absorbance at 490 nm.

FIG. 34A through FIG. 34D are sensograms (the results of BIACore™ PSR measurement) of PSA to Siglecs 7, 9 and 13.

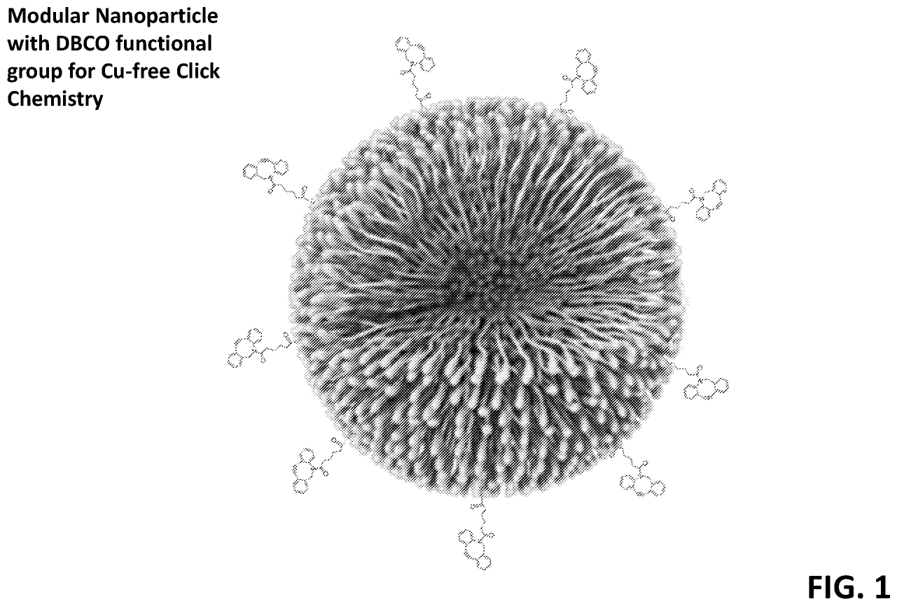

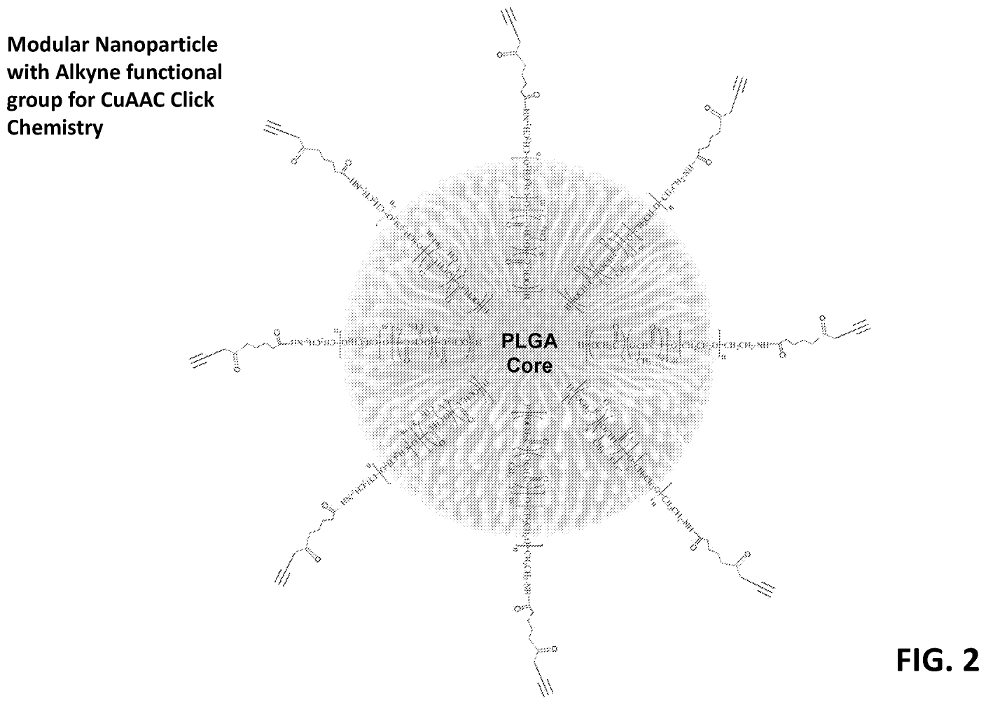

### DETAILED DESCRIPTION

As used herein, a “sialic acid ligand” refers to any monosaccharide or polysaccharide derivative of a sialic acid that is cognate to at least one of the sialic acid receptors. A sialic acid refers to neuraminic acid or any chemical modification of neuraminic acid, either naturally occurring or synthetically derived. The structural formula of neuraminic acid is reproduced below:

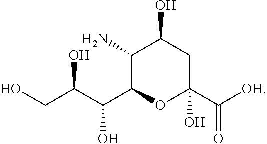 [SMILES: N[C@H]1[C@H]([C@H](O)[C@H](O)CO)O[C@](O)(C(=O)O)C[C@@H]1O]

Examples of a sialic acid derivative include N-acetylneuraminic acid (Neu5Ac), represented by the following structural formula:

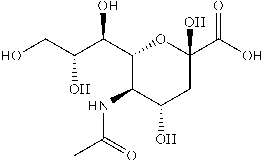 [SMILES: CC(=O)N[C@H]1[C@H]([C@H](O)[C@H](O)CO)O[C@](O)(C(=O)O)C[C@@H]1O]

and N-Glycolylneuramiyc acid (Neu5Gc), represented by the following structural

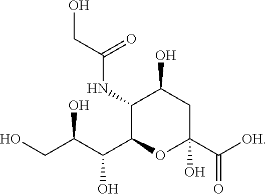 [SMILES: O=C(CO)N[C@H]1[C@H]([C@H](O)[C@H](O)CO)O[C@](O)(C(=O)O)C[C@@H]1O]

As used herein, a carbohydrate residue is a monosaccharide in which one or more positions are modified for covalent linkage.

As used herein, an “infectious agent” is a viral, bacterial, or a parasitic agent, and the receptor can be a capsid/capsule, membrane or nuclear glycan binding molecules/proteins/enzymes (lectins) such as hemagglutinin esterase, coronavirus spike protein, viral neuraminidase/sialidase.

As used herein, an “average cross-sectional width” is the widest part in a non-spherical nanoparticle, averaged over an ensemble of particles.

As used herein, the term “particle” includes a microparticle and a nanoparticle, as defined herein.

The present disclosure provides therapeutic agents comprising sialic-acid ligands for use as immune system modulators, i.e., suppressors or activators of the immune system, inhibitors of viral/bacterial/parasitic infectivity, unmasking of cancer cells or damage-associated molecular patterns (DAMPs) to enhance immune surveillance. Target cell populations include those expressing Siglec receptors. CFH CCP 4-6, 19-20, viral HE, viral N, viral SP, and CD147. In a specific embodiment, the delivery vehicles comprise polymers formulated as nanoparticles or microparticles, tethered (conjugated or linked) to ligands comprising sialic acids, sialic-acid derivatives, sialic-acid analogs, sialic-acid monomers, and/or sialic-acid polymers (collectively referred to herein as “sialic-acid ligands”) for presentation on the nanoparticle surface. The tethered sialic acid functions as a ligand for targeted binding of the nanoparticle to receptors, such as Siglec receptors, expressed on the surface of targeted cells.

In one aspect, the present disclosure provides nanoparticles comprising polymers that provide for tethering via covalent chemical conjugation of sialic-acid ligands for presentation on the nanoparticle surface. The nanoparticles can be used to contact immune cells expressing sialic-acid-binding immunoglobulin-type lectins (Siglecs) in order to modulate inflammatory processes. It has been determined that providing sialic-acid ligands, capable of targeting and binding to immune cells expressing sialic-acid-binding immunoglobulin-like lectins (Siglecs) can be used to modulate an inflammatory response in the targeted cells and associated environment. Siglecs are members of the self-associated pattern recognition family of receptors and include Siglec isotypes that are expressed selectivity on different cell populations. Accordingly, the ability to design nanoparticles that bind selectively to specific Siglec receptors allows one to target binding to a desired cell population of interest. Such binding of the nanoparticle to the Siglec receptor may be used as a means for modulating the signal transduction activity of the Siglec receptor within the cell of interest, resulting in a decrease in inflammatory responses or enhancement of anti-inflammatory responses in a treated subject.

Presentation of a sialic-acid ligand on a nanoparticle surface means that the sialic-acid ligands are decorated on the nanoparticle such that they are available to be bound by a Siglec receptor on a target cell, or organism. Suitably they may be provided to bind, activate or block the receptor. Without wishing to be bound by theory, the presentation of the sialic-acid on a nanoparticle requires the sialic-acid ligand to be presented at a specific concentration density in order to modulate inflammatory response, enhance immune surveillance or block infectivity.

A single nanoparticle may be decorated with a multivalent complex of sialic-acid ligands, which will allow for multivalent binding of different Siglec receptors by this single nanoparticle resulting in modulation of the inflammatory response. Nanoparticles decorated with a unique ligand can also be mixed with other ligand-decorated nanoparticles that may target different Siglec receptors, again enabling desired modulation of the inflammatory response. In some embodiments, the presentation of sialic-acid ligand on the surface of a nanoparticle, or microparticle, can provide for an increased uptake of the particle by a cell of at least about two-fold, at least about three-fold, at least about four-fold, at least about five-fold, at least about six-fold, or at least about 10-fold. In some embodiments, the presentation of sialic-acid ligand on the surface of nanoparticle or microparticle can decrease an inflammatory response. In a non-limiting embodiment, the decrease in an inflammatory response is over about two-fold, over about three-fold, over about four-fold, over about five-fold, over about 10-fold, over about 20-fold, over about 50-fold, over about 100-fold, over about 500-fold or over about 1000-fold.

The nanoparticle or microparticles may be used for systemic delivery or local delivery to target diseased tissues in a subject in need of treatment resulting in modulation of an inflammatory response in said subject to resolve innate and adaptive inflammation, activate innate and/or adaptive immunity when enhanced immune surveillance is desired, or reduce infectivity of infectious organisms. The targeted immune cells or virus should possess Siglec receptors or viral sialic-ligand binding regions, respectively. The activity of the innate immune system includes, for example, the cellular response of the innate immune system: the non-cellular/humoral response of the innate immune system, the complement system, the alternative complement pathway, the amplification loop of the alternative complement pathway, and/or the amplification loop of the alternative complement pathway activated by complement factor H. The activity of the adaptive immune system involves dendritic cell maturation and presentation to T cells, T-cell activation, T-cell modulation, T-cell checkpoint inhibition or activation, neutrophil NETosis, and B-cell activation. The reduction of infectivity includes reduction of viral ingress into host cells, reduction in reproduction of viral particles, or reduction in inflammatory response to the viral infection.

As used herein, “subject” refers to the subject being treated according to the provided treatment methods. A subject can be human, a primate, canine, feline, bovine, equine, murine, etc. Subject also refers to those animals being used for laboratory testing.

As used herein, “nanoparticle” refers to a particle, composed of one or more polymers, whose size in nanometers (nm) includes a range of linear dimensions between 10 nanometers to 2000 nanometers. As used herein, “linear dimension” refers to the distance between any two points on the surface of a nanoparticle measured in a straight line. Nanoparticles of the present disclosure can be irregular, oblong, spindle, rod, cylindrical, pancake, discoid, spherical, biconcave, or red blood cell shaped. Linear dimension can be measured using multiple methods including but not exclusive to transmission electron microscopy or tunable resistive pulse sensing which are some of the standard means of determining nanoparticle size. One of the widely used techniques for measuring the size of nanoparticles is dynamic light scattering (DLS) that can provide the diameter and polydispersity of the nanoparticles. DLS assumes that the nanoparticles are spherical in nature, and the size of the nanoparticles are the average diameter (or radius) of such assumed spheres. In such measurements, the nanoparticles can be described to have a size range of 10 nm to 1000 nm or 1 nm to 500 nm.

As used herein, “microparticle”, refers to a microscopic particle, composed of one or more polymers, whose size in micrometers (μm) includes a greatest cross-sectional width less than 1000 μm and which is greater than or equal to 1 μm.

Several types and configurations of nanoparticles are encompassed by the present disclosure. For example, nanoparticles may be composed of a range of materials including, but not limited to, a biodegradable polymer, biocompatible polymer, a bioabsorbable polymer, or a combination thereof. Biocompatible refers to polymers that do not undesirably interfere with biological function of tissues. The terms biodegradable, bioabsorbable, and bioerodible, as well as degraded, eroded, and absorbed, are used interchangeably (unless the context shows otherwise) and refer to polymers and metals that are capable of being degraded or absorbed when exposed to bodily fluids such as blood, and components thereof such as enzymes, and that can be gradually resorbed, absorbed, and/or eliminated by the body.

The polymer backbone of the nanoparticle, upon which the sialic-acid ligands are linked, may be composed of naturally occurring polymers, such as carbohydrates or proteins, or may be composed of synthetic polymers. The polymer backbone will have a unique terminal functional group to provide for tethering of the sialic-acid ligand to the nanoparticle surface. The polymer backbone may first be joined with a plurality of sialic-acid ligands prior to forming the nanoparticle via chemical conjugation methods, or the polymer backbone may first be formed into a nanoparticle and then the functional groups displayed on the surface of the nanoparticle can be joined with sialic-acid ligands via chemical conjugation methods.

Suitable nanoparticles include polymer particles and hydrogel particles. As used herein, a “polymer” refers to a molecule(s) composed of a plurality of repeating structural units connected by covalent bonds. As used herein, a “polymer particle” refers to a solid or porous particle in contrast to the shell-like structure of liposomes and polymersomes and the relatively open structures of hydrogel particles. As used herein, a “hydrogel particle” refers to a cross-linked network of polymer chains that is absorbent but stable in an aqueous environment.

Polymers that may be used to prepare nanoparticles include, but are not limited to, poly(N-acetylglucosamine) (Chitin), Chitosan, poly(3-hydroxyvalerate), poly(D,L-lactide-co-glycolide), poly(1-lactide-co-glycolide) poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), Poly((D,L)Lactide)-b-Poly(ethylene glycol)-Azide, Poly(DL-lactide)-b-poly(ethylene glycol)-methyltetrazine, poly(D,L-lactide), poly(L-lactide-co-D,L-lactide). Poly(D,L-lactide)-b-poly(ethylene glycol)-carboxylic acid, Poly(ethylene glycol) methyl ether-block-poly(lactide-co-glycolide), Poly(lactide-co-glycolide)-b-poly(ethylene glycol)-b-poly(lactide-co-glycolide), Poly(lactide-co-glycolide)-b-poly(ethylene glycol)-azide, Poly(lactide-co-glycolide)-b-poly(ethylene glycol)-alkyne, Poly((D,L)Lactic acid)-b-Poly(ethylene glycol)-Azide. Poly((D,L)Lactic acid)-b-Poly(ethylene glycol)-alkyne, poly(caprolactone), Poly(caprolactone)-b-Poly(ethylene glycol), Polycaprolactone-b-poly(ethylene glycol), Poly(lactide-co-caprolactone)-b-poly(ethylene glycol)-b-poly(lactide-co-caprolactone), poly(L-lactide-co-caprolactone), Poly(L-lactide-co-caprolactone), poly(D,L-lactide-co-caprolactone), poly(glycolide-co-caprolactone), Poly(DL-lactide)-b-Poly(ethylene glycol)-b-Poly(DL-lactide), poly(trimethylene carbonate), polyester amide, poly(glycolic acid-co-trimethylene carbonate). Acrylate-Poly(caprolactone)-b-Poly(ethylene glycol)-alkyne, co-poly(ether-esters) (e.g. PEO/PLA), Poly(N-isopropylacrylamide-co-acrylic acid), Poly(N-isopropylacrylamide-co-methoxy poly(ethylene glycol) methacrylate), poly phosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), poly(vinylidene fluoride), poly(vinylidene fluoride-co-hexafluoropropylene), polyacrylonitrile, poly vinyl ketones, poly vinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, and fullerenes.

In one aspect, the nanoparticles are formed from a biodegradable polymer polycaprolactone, and in other embodiments formed of a polymer comprising polyglycolic acid, poly(L-lactic acid), poly(lactic-co-glycolic acid), polycaprolactone, poly(3-hydroxybutyric acid), In embodiments, the nanoparticle may be a polymeric particle, in particular a particle may be formed from a biodegradable polyester such as poly(lactide) (PLA), poly(glycolide)(PGA), poly lactic-10-glycolic acid (PLGA), poly(butyl cyanoacrylate) (PBCA), or N-(2-hydroxypropyl)methacrylamide (HPMA) copolymers. In another aspect, the nanoparticles are formed from a nonbiodegradable polymer such as poly(ethylene glycol), polyethylene oxide, Pluronic F127, Pluronic F68, poloxamer, poly(hydroxymethylmethacrylate), polyvinyl alcohol and poly(vinylpyrrolidone).

In some embodiments, the nanoparticles are formed from mixtures of biodegradable and nonbiodegradable polymers as block copolymers (BCPs), including a preferred embodiment of PLGA-block-PEG. A block copolymer comprises a polymer having two or more different polymer subunits linked by covalent bonds. In some embodiments the nanoparticles are formed from mixtures of biodegradable and nonbiodegradable polymers as block copolymers, including a preferred embodiment of PLGA-block-PEG. In yet another aspect, the nanoparticles are formed from naturally derived polymers in the form of hydrogel nanoparticles including formed from collagen, hyaluronic acid, heparin, heparin sulfate, chitosan, and alginate.

Methods for synthesis of nanoparticles are well known to those of skill in the art. (see, for example, Spence et al., Science Translational Medicine, 2015, 7: 303 303ra140 and references cited therein), for example, methods for synthesis of nanoparticles with known degradation rates are known to those skilled in the art, as described in U.S. Pat. No. 6,451,338 to Gregoriadis et al., U.S. Pat. No. 6,168,804 to Samuel et al. and U.S. Pat. No. 6,258,378 to Schneider et al., which are hereby incorporated by reference in their entirety.

Formulated nanoparticles or microparticles, tethered with sialic-acid ligands for use in selective binding to receptors expressed on target cells of interest are provided. The term sialic acid refers to any monosialic-acid, oligomeric sialic-acid, or polymeric sialic-acid or polysialic-acid, including disialic-acids which can bind to a Siglec receptor, in particular a sialic acid with binding specificity to inhibitory Siglec receptors, such as for example Siglec 7. In embodiments, a sialic acid for use in the presently disclosed compositions or methods can be any group of amino carbohydrates that are components of mucoproteins and glycoproteins in animal tissue and blood cells. In embodiments sialic acids (also known as nonulosonic acids) are members of a family of amino containing sugars containing nine or more carbon atoms, for example, N-acetylneuraminic acid (also known as 5-(acetylamino)-3,5-dideoxy-D-glycero-D-D-galacto-nonulosonic, lactaminic acid and O-sialic-acid).

In embodiments, it is envisaged that the sialic acids can be monosialic acids, or polysialic acids, including disialic acids. Polysialic acids may be linked 2→8 and/or 2→9, and/or 2→6, and/or 2→3, usually in the α-configuration. In embodiments wherein sialic acids or polysialic acids are tethered to the surface of the nanoparticle or microparticle. Polysialic acid is a homopolymer comprising of multiple sialic acid units. Polysialic-acid, maybe less than five sialic-acid units, preferably less than four sialic-acid units, less than three sialic-acid units long, and, most preferably, two sialic-acid units in length. In embodiments, the degree of polymerization (DP) may range from DP2 to over DP100. The DP may be between DP2 and DP100, between DP2 and 90, between DP2 and DP80, between DP2 and DP70, between DP2 and DP60, between DP2 and DP50, between DP2 and DP40, between DP2 and DP30, between DP2 and DP30, between DP2 and DP20, between DP2 and DP10. In a specific non-limiting embodiment, the the degree of polymerization is from DP3 to DP100. In alternative embodiments, polysialic acid can comprise five or more sialic-acid units. For example, a polysialic acid can comprise at least six sialic-acid units, at least seven sialic-acid units, or at least eight sialic-acid. In embodiments, the degree of polymerization (DP) may range from DP5 to DP1000. For example, the degree of polymerization can be from DP5 to DP500, from DP5 to DP100, from DP5 to DP90, between DP5 to DP80, from DP5 to DP70, from DP5 to DP60, from DP5 and DP50, from DP5 to DP40, from DP5 to DP30, from DP5 to DP20, from DP5 to DP10. In a specific non-limiting embodiment, the the degree of polymerization is from DP10 to DP400, from DP20 to DP300, or from DP30 to DP 200. In certain embodiments, the DP is from DP5 to DP30, for example, DP5, DP10, DP15, DP20, DP25, DP30, DP35, DP40, DP45 or DP50. In certain embodiments, DP is from DP5 to DP500. In example embodiment, DP is from DP10 to DP30.

In embodiments wherein an analog of sialic acid is used, the analog may have structural similarity to sialic acid as disclosed herein and have binding affinity to certain Siglecs. Suitable analogs would be known in the art. It is believed that the feature which influences binding of a sialic-acid ligand to a Siglec receptor is the charge-distance-coordination relationship between the carboxylic acid functionality of sialic acid.

In specific embodiments, the sialic acids are selected from NeuAcα2-3Galβ1-4Glc, NeuAcu2-3Galβ1-4GlcNAc, NeuAcα2-3Galβ1-3GlcNAc, NeuAcα2-3Galβ1-3GalNAc, NeuGcα2-3Galβ1-4GlcNAc, NeuGcα2-3Galβ1-3GlcNAc, NeuAcα2-6Galβ1-4Glc, NeuAcα2-6Galβ1-4GlcNAc, NeuAcα2-6GalNAc, Galβ1-3(NeuAcα2-6)GalNAc, NeuGcα2-6Galβ1-4Glc, NeuGcα2-6Galβ1-4GlcNAc, NeuGcα2-6GalNAc, NeuAcα2-8NeuAcα2-3Galβ1-4Glc, NeuAcα2-6Galβ1-4GlcNAc, NeuAcα2-3Galβ1-4[Fucα1-3]GlcNAc, NeuAcα2-6Galβ1-4GlcNAc6S, NeuAcα2-3Galβ1-4GalNAc, NeuAcα2-8NeuAc, NeuAcα2-3GalβSβ1-4GlcNAcα2-3Fuc, NeuAcα2-3Galβ1-4GlcNAc6Sα2-3Fuc, and NeuAcα2-8NeuAc or sialoside derivatives of such sialic acids, for example BPCNeuAc sialosides.

Many other synthetic/unnaturally occurring sialioside/sialic-acid derivatives are available. The sialic acids can be obtained from high throughput screening or cell-based microarrays to obtain the highest affinity binding sialic-acid analogs/derivatives for the respective Siglec receptor.

In embodiments, a sialic-acid for use in the present disclosure can be selected according to the Siglec receptor being targeted wherein the binding preference for particular Siglecs may be selected from Table 1.

TABLE 1

| | |
|---|---|
| Siglec 1 | NeuAcα2-3Galβ1-4GlcNAc |
| Siglec 2 | NeuAcα2-6Galβ1-4GlcNAc6S |
| Siglec 3 | NeuAcα2-6Galβ1-4GlcNAc |
| Siglec 4 | NeuAcα2-3Galβ1-4GalNAc |
| Siglec 5 | NeuAcα2-8NeuAc |
| Siglec 6 | NeuAcα2-8NeuAc |
| Siglec 7 | NeuAcα2-8NeuAcα2-3Galβ1-4Glc |
| Siglec 8 | NeuAcα2-3Gal6Sβ1-4GlcNAcα2-3Fuc |
| Siglec 9 | NeuAcα2-3Galβ1-4GlcNAc6Sα2-3Fuc |
| Siglec 10 | NeuAcα2-6Galβ1-4GlcNAc |
| Siglec 11 | NeuAcα2-8NeuAc |
| Siglec 11 | α2,8-linked polysialic-acid |
| Siglec 14 | NeuAcα2-8NeuAc |
| Siglec 15 | NeuAcα2-6GalNAc |
| Siglec 16 | NeuAcα2-8NeuAc |

In some embodiments, appropriate sialic-acid ligands or analogs can be conjugated to the nanoparticle according to the receptor specificity desired. In some embodiments, sialic acid, is at least one of 2-8 disialic-acid, or 2-6 or 2-3 variants with at least one position filled with a sialic-acid. In some embodiments, the sialic acid can be selected from at least one of alpha 2-8 di-acetylneuraminic acid, alpha-NeuNAc-(2→6)-β-D-Gal-(1→4)-D-Glc and alpha-NeuNAc-(2→3)-β-D-Gal-(1→4)-D-Glc. In particular embodiments, the nanoparticle can be provided with alpha 2-8 acetylneuraminic acid.

Sialic acids naturally bind to cells of the immune system via Siglec receptor interactions and can modulate the activity of said cells through binding. Accordingly, the nanoparticle disclosed herein can be designed to bind to specific cells of the immune system based on their binding affinity to specific Siglecs expressed on the surface of immune cells. For example, the activity of macrophages can be modulated by sialic-acid ligand bearing nanoparticles that bind to and agonizes Siglec 3, Siglec 5, Siglec 7, Siglec 9, Siglec 11, Siglec 12, Siglec 15, or bind to and antagonize Siglec 16, in each case such Siglecexpressed on the surface of macrophages. Potential therapeutic biological response is a polarization of the macrophage to M2c the resolution anti-inflammatory state for macrophages or M0 which is the sensescent state of macrophages. In another example, the activity of monocytes can be modulated by sialic-acid ligand bearing nanoparticles that bind to and agonizes Siglec 3, Siglec 5, Siglec 7, Siglec 9, Siglec 10, Siglec 13, or bind to and antagonize Siglec 14, in each case such Siglec expressed on the surface of monocytes. The biological response is inhibition of cytokine production of monocytes such as interleukin-1 beta (IL-Ibeta), IL-6, IL-8, IL-10, IL-12p40, IL-12p70, IL-23, IL-23p40, CCL17, CXCL10, MCP-1, tumor necrosis factor-alpha (TNF-alpha), and transforming growth factor-beta (TGF-beta1), interferon gamma (IFNγ). In another example, the activity of NK cells can be modulated by sialic-acid bound nanoparticles designed to bind to Siglec 7 expressed on the surface of NK cells. The biological response is inhibition of pyroptosis. Markers of pyroptosis are the downregulation of NOD-, LRR- and pyrin domain-containing protein 3 (NLRP3), down regulation of IL-Ibeta and reduction of granzyme B. In another example, the activity of eosinophils can be modulated by sialic-acid bound nanoparticles that bind and agonizes Siglec 7, Siglec 8 (including its murine homolog Siglec F), or Siglec 10, expressed on the surface of eosinophils. The biological response is induced cell death of eosinophils, reduction in mast cell degranulation, and reduction in histamine production. In another example, the activity of neutrophils can be modulated by sialic-acid bound nanoparticles designed to bind and agonize Siglec 3, Siglec 5, Siglec 9 or bind to and antagonize Siglec 14, in each case such Siglec expressed on the surface of neutrophils. The biological response is inhibition of NETosis. Markers of NETosis inhibition are the downregulation of neutrophil elastase, cathepsin G, lactoferrin and gelatinases. In another example, he activity of microglia of the central nervous system or dendritic cells in other tissues can be modulated by sialic-acid ligand bound nanoparticles designed to bind and agonize Siglec 3, Siglec 7, or Siglec 9, in each case such Siglec expressed on the surface of microglia of the central nervous system or dendritic cells. The biological response is upregulation of CD80, CD83, and/or CD 86 maturation and antigen presenting markers. In another example, the activity of B cells can be modulated by sialic-acid ligand bound nanoparticles designed to bind and agonize Siglec 2, Siglec 5, or Siglec 10, in each case such Siglec expressed on the surface of B cells. The biological cytokine response is inhibition of lymphotoxin, Il-6, interferon-gamma, and TNF. The biologic cell marker response is a downregulation of IGM formation, reduction of class switch DNA recombination, and/or reduction of class switched plasma cells. In another example, the activity of CD8+ T cells can be modulated by sialic-acid ligand bound nanoparticles designed to bind and agonize Siglec 7 or Siglec 9, in each each case such Siglec expressed on the surface of T cells. The biological cytokine response is inhibition of lymphotoxin. Il-6, interferon-gamma, and/or TNF. The biological response is inhibition of interferon-gamma, TNF, LAG-3 or CD160. The biologic cell marker response is a downregulation of 2b4, and PD-1. In another example, the activity of CD34+ T cells can be modulated by sialic acid ligand bound nanoparticles designed to bind and agonize Siglec 3, Siglec 5, Siglec 9, or Siglec 10, in each case such Siglec expressed on the surface of T cells. The biological cytokine response is inhibition of lymphotoxin. Il-6, interferon-gamma, and TNF. The biological response is inhibition of interferon-gamma, TNF, LAG-3 or CD160. The biologic cell marker response is a downregulation of 2b4, and PD-1. In another example, the activity of mast cells can be modulated by sialic-acid ligand bound nanoparticles that bind and agonize Siglec 3, Siglec 5, Siglec 6, or Siglec 8 (including its murine homolog Siglec F), in each case such Siglec expressed on the surface of eosinophils. The biological response is induced cell death of mast cells, reduction in mast cell degranulation and reduction in histamine production. In another example, the activity of basophils can be modulated by sialic-acid ligand bound nanoparticles that bind and agonize Siglec 3, Siglec 5, Siglec 6, or Siglec 8 (including its murine homolog Siglec F), in each case such Siglecexpressed on the surface of basophils. The biological response is induced cell death of basophils, reduction in mast cell degranulation, and reduction in histamine production. In another example, the activation of the alternative complement cascade can be modulated by a nanoparticle that contains a sialic-acid ligand that bind to CFH at the CCP region 4-6 and/or 19-20 and hyperactivates the function of both CFH and CFH with the Y402H polymorphism. The biological response would be the reduction in the formation of membrane attack complex and other complement factors such as C3bBb or C5 and/or an increase in the cleaved C3bBb breakdown products in both the wild type and the Y402H polymorphic CFH. Other downstream measures of CFH activation include the ability to reduce lysis in the sheeps RBC model of complement activation. In another example, the infectivity of a virus can be modulated by a nanoparticle that contains a sialic-acid ligand that bind neuraminidase or sialidase and is capable of inhibiting the cleavage of neuraminic/sialic-acid by the neuramnindase/sialidase expressed by a virus. This should reduce the release of viral particles in infections with influenza A, B, and C. In another example, the infectivity of a virus can be modulated by a nanoparticle that contains a sialic-acid ligand that bind to sialic-acid binding sites found on the capsid of viruses such as influzenzaA, influenza B, influenza C, SARS-CoV1, or SARS-CoV2's respective spike proteins. This should reduce the ability of these viruses to bind and infect respiratory tract cells or other host cells by blocking, interfering with, or modulating virus attachment or docking molecules and blocking their ability to bind and infect such host cells. In another example, the reduction in pulmonary inflammation can be modulated by a nanoparticle that contains a sialic-acid ligand that bind neuraminidase or sialidase and is capable of inhibiting the cleavage of neuraminic/sialic-acid by the neuramindase/sialidase expressed by epithelial cells of the bronchial, alveolar, and or respiratory tract. This should reduce the inflammation caused by degradation of the constitutive cis binding sialic-acid that bind to native pulmonary Siglecs, which are cleaved by sialidase and result in constitutive anti-inflammation. In another example, the activity of tumor associated macrophages can be modulated by sialic-acid ligand bearing nanoparticles that bind to and antagonize Siglec 3, Siglec 5, Siglec 7, Siglec 9, Siglec 11, Siglec 12, Siglec 15, or bind to and agonize Siglec 16, in each case such Siglec expressed on the surface of macrophages. Potential therapeutic biological response is a polarization of the macrophage to M1 and M2a, b, or d in order to unmask cancer cells from using tumor Siglec agonism to evade immune surveillance. The macrophage cellular response is an increase in phagocytosis and cytokine killing and destruction of tumor cells. In another example, the activity of tumor associated monocytes can be modulated by sialic-acid ligand bearing nanoparticles that bind to and antagonize Siglec 3, Siglec 5, Siglec 7, Siglec 9, Siglec 10, Siglec 13, or bind to and agonize Siglec 14, in each case such Siglec expressed on the surface of monocytes. The biological response is enhancement of cytokine production of monocytes such as IL-Ibeta, IL-6, IL-8, IL-10, IL-12p40, IL-12p70, IL-23, IL-23p40, CCL17, CXCL10, MCP-1, tumor necrosis factor-alpha (TNF-alpha), and transforming growth factor-beta (TGF-beta1), interferon gamma (IFNγ), which indicates the restoration of immune surveillance as it relates to attacking cancer cells that utilize Siglec agonism to escape tumor surveillance. Mononcyte cellular response will be an increase in phagocytosis and cytokine killing and destruction of tumor cells. In another example, the activity of tumor-associated NK cells can be modulated by sialic-acid bound nanoparticles designed to bind and antagonize Siglec 7 expressed on the surface of such NK cells. The biological response is enhancement of pyroptosis. Markers of pyroptosis are the upregulation of NLRP3, upregulation IL-1 beta, upregulation of granzyme B, activation of toll-like receptors 1-4. NK cellular response is an increase in pyroptotic cytokine killing and destruction of tumor cells. In another example, the activity of tumor-associated CD8+ T cells can be modulated by sialic-acid ligand bound nanoparticles designed to bind and antagonize Siglec 7 or Siglec 9, in each case such Siglec expressed on the surface of tumor associated T cells. The biological cytokine response is upregulation of lymphotoxin, Il-6, interferon-gamma, and TNF in the presence of cancer cells. The biologic cell marker response is a upregulation of 2b4, LAG-3, CD160 and PD-1. The T cell biologic response will be an increase in the activation of the adaptive immune system to attack and kill tumor cells. In another example, the activity of CD34+ T-cells can be modulated by sialic bound nanoparticles designed to bind and antagonize Siglec 3, Siglec 5, Siglec 9, or Siglec 10, in each case such Siglec expressed on the surface of T cells. The biological cytokine response is upregulation of lymphotoxin, IL-6, interferon-gamma, and TNF in the presence of cancer cells. The biologic cell marker response is a upregulation of 2b4, LAG-3, CD160, and PD-1. The T cell biologic response is an increase in the activation of the adaptive immune system to attack and kill tumor cells. In another example, the activity of osteoclasts can be modulated by sialic-acid ligand bound nanoparticles that bind to Siglec 15 expressed on the surface of osteoclasts.

Table 2 presents Siglecs and the cells types on which they are most commonly expressed, as well as an effective type of sialic-acid linkages for each Siglec.

TABLE 2

| | | Preferred |
|---|---|---|
| | | Sialic-acid |
| Siglec | Cell Types | Linkage |
| 1 | Macrophages | α2-3, α2-6 |
| 2 | B cells | α2-6 |
| 3 | Dendritic Cells, Myeloid progenitors, | α2-3, α2-6 |
| | Macrophages, Monocytes, Neutrophils, | |
| | Basophils, Mast Cells, Microglia | |
| 4 | Oligodendrocytes, Schwan Cells | α2-3 |
| 5 | Monocytes, Neutrophils, Basophils, B Cells | α2-8, 2-6 |
| 6 | Basophils, Mast Cells, Trophoblast | α2-6 |
| 7 | NK Cells, Dendritic Cells, Monocytes | α2-8, α2-6, |
| | | α2-3 |
| 8 | Eosinophils, Basophils, Mast Cells | α2-3, α2-6 |
| 9 | CD8+ T Cells, Dendritic Cells, Monocytes, | α2-3, α2-6 |
| | Macrophages Neutrophils, | |
| 10 | B Cells, Monocytes, Eosinophils | α2-3, α2-6 |
| | Macrophages, Microglia | α2-8 |
| 12 | Macrophages | ND |
| 13 | Monocytes* | ND |
| 14 | Neutrophils, Monocytes | 2-8. 2-6 |
| 15 | Macrophages, Osteoclasts | α2-6 |
| 16 | Macrophages | α2-8 |
| 17 | NK Cells | α2-6 |
| E | Neutrophils, Monocytes, Dendritic Cells | 2-3, 2-6, 2-8 |
| F | Eosinophils | 2-3, 2-6 |
| G | B Cells | 2-3, 2-6 |
| H | Dendritic Cells | ND |

*Expressed in chimpanzee
ND = Not Determined
Siglec E, F, G, and H are expressed in mice

The sialic-acid ligands tethered to the nanoparticle surface may be in the form of monomers, oligomers, or polymers. In certain embodiments, the sialic-acid ligands may be first joined onto a polymer backbone via chemical conjugation techniques, and then subsequently the polymer-conjugated-sialic-acid ligand construct can be formed into to the nanoparticle surface. In other embodiments, the polymers are formed into nanoparticles with exposed functional groups on the nanoparticle surface, such that these functional groups can be conjugated with sialic-acid ligands.

In another embodiment, screening methods may be employed to identify a nanoparticle that binds specifically to one or more Siglec receptor(s). Such identified nanoparticles may then be tested to determine whether said binding to the Siglec receptor induces a detectable cellular response

In another embodiment, a screening library and high throughput screening method is provided based on the use of a plurality of nanoparticles, wherein the linked sialic-acid ligands of the nanoparticles are varied. Such libraries may be used in methods for identifying a nanoparticle that binds specifically to a Siglec receptor comprising contacting the nanoparticle library to a cell expressing said a Siglec receptor or a support comprising a Siglec receptor. Once binding of a member of the nanoparticle library to a Siglec receptor has been identified, the nanoparticle may be tested to determine whether said binding to the Siglec receptor induces a detectable cellular response. In certain embodiments, the polymers are formed into nanoparticles with exposed functional groups on the nanoparticle surface, such that these functional groups can be used to tether the sialic-acid ligands to the nanoparticle surface. As used herein, “modular” nanoparticle refers to a nanoparticle having a specific functional group that can be used as a universal platform for joining one or more sialic-acid ligands to the nanoparticle in a desired manner.

Sialic-acid ligands comprising oligomers and polymers may be joined together by any combination of a2-3, a2-6, a2-8, or a2-9 glycosidic linkages. The type of glycosidic linkages joining the sialic acids or sialic-acid analogs to the nanoparticle surface can be controlled to maximize binding affinity to target Siglec receptors and enhance specificity for a particular Siglec. As different Siglecs are known to be differentially expressed by different cell types, the selection of specific types of sialic-acid linkages can be used to determine the type of cells to be contacted, or targeted, by the nanoparticles with the sialic-acid ligands. Sialic-acid ligands in the form of oligomers and polymers may have linear or branched structures. The branched structure of the oligomer or polymer form may be created by introduction of a glycosidic linkage different from adjacent glycosidic linkages. The oligomer and polymer forms may be homogeneous in composition, composed of a one type of sialic-acid, or they may be heterogeneous in composition, composed of a plurality of sialic-acid. The oligomer and polymer forms may also be comprised of other carbohydrate monomers, such as galactose, N-acetylgalactoseamine, glucose, N-acetylglucoseamine, mannose, N-acetylmannosamine, fucose, or other sugar/carbohydrates in addition to sialic-acids and/or sialic-acid analogs.

The sialic-acids may be naturally derived (e.g., Neu5Ac, Neu5Gc, Neu5Ac9Ac, etc) or may include any synthetically prepared sialic-acid analogs. Sialic-acid analogs are known in the art. In embodiments, such analogs can have substitutes at position C9. Analogs can also have substitutes at C1, C4, C5, C7, and C8. Analogs can include neuranuinic acid derivatives, sialosides, and any sugars comprising at least one neuraninic acid molecule.

The sialic analogs may be prepared by means of chemical synthesis, chemoenzymatic synthesis (e.g., one-pot multienzyme; OPME), or via mammalian or bacterial cellular synthesis such as by cell feeding of precursor carbohydrates (e.g., mannose derivatives), recombinant methods, or genetic engineering methods. The sialic-acid analogs prepared for use as nanoparticle ligands may be prepared using one-pot synthesis or microarray platform. Arrays of sialic-acid analog ligands can be prepared in-situ using HTS methods.

Chemical linkage of the sialic-acid ligand to the nanoparticle surface may be achieved through the use of click chemistry reactions. In such reactions, a chemical reaction occurs between a terminal functional group of a nanoparticle polymer and a terminal functional group of a sialic-acid ligand (referred to herein as “terminal functional group conjugate pairs”) resulting in linkage of the polymer and sialic-acid ligand. The types of terminal functional groups found on the surface of the polymer, and its binding partner ligand, will determine the type of click chemistry reaction that is to be used to chemically link the sialic-acid ligand to the surface of the nanoparticles. Additionally, the selection of polymers having specific terminal functional groups can be used to control the types, density and spatial arrangement of sialic-acid ligand conjugate partners to be presented on the surface of the nanoparticle. In embodiments, the polymers have specific functional groups that provide chemical conjugation sites on the formed nanoparticle surface including azide, alkyne, aryl ester, amide, amine, aryl amide, aldehyde, acetyl, substituted aryl ester, alkyl ester, alkyl ketone, aryl ketone, substituted aryl ketone, ketone, alkyl halide, amnioxy, alcohols, aza-ylide, carboxylic acid, ester, amide, bicyclononyne, dihydrazide, halo-carbonyl, halosulfonyl, hydrazide. N-hydroxysuccinimide, succinimidyl ester, monofluorinated and difluorinated cyclooctynes, isothiocyanate, iodoacetamide, maleimide, methylcyclopropene, hydrazine, nitrile, nitro, phosphine, phosphazide, tertazine, methyl-tetrazine, trans-cyclooctene, strained alkynes, dibenzocyclooctyne, biarvlazacyclooctynone, azadibenzvlcyclooctyne, vinyl, sulfonyl ester, thioester, thiocarboxylate, thioester a sulfonyl halide, thiol, and thiolene.

Such conjugation sites provide a position for linkage of the sialic-acid ligands to the surface of the nanoparticle through performance of click chemistry reactions. In one example, the nanoparticle is formed of a PLGA-PEG polymer with an azide or alkyne terminal functional group. In a non-limiting embodiment, blends of different polymers having different terminal functional groups may be used. Such polymers include, for example, PLGA-PEG-alkyne, PLGA-PEG-ester and PLGA-PEG-DBCO. In a specific aspect, a blend of PLGA-PEG-alkyne and PLGA-PEG-carboxylic acid may be prepared as nanoparticles. In another specific aspect, a blend of PLGA-PEG-akyne and PLGA-PEG-ester may be prepared as nanoparticles. In another specific embodiment, a blend of PLGA-PEG-DBCO and PLGA-PEG-carboxylic acid may be prepared as nanoparticles. In another specific aspect, a blend of PLGA-PEG-DBCO and PLGA-PEG-ester may be prepared as nanoparticles.

Although PLGA polymers can possess free terminal alkyne groups, many of these can be buried in the particle matrix and not be available for binding on the surface of the particle. In some embodiments, more alkyne groups may be introduced to the particle by providing a second polymer or copolymer surfactant or coating in addition to the first PGLA polymer or copolymer of the particle. Suitably the second polymer or copolymer can be branched or linear and can have a plurality of terminal alkyl groups wherein an alkyl group contains only carbon and hydrogen and forms the homologous series with the general formula CnH2n+1. In other embodiments, the sialic-acid ligands or analogs can be attached to the particle, for example a polymeric nanoparticle, via a covalent linkage.

In other embodiments, the sialic-acid ligands comprise terminal functional groups (i.e., conjugation sites) that provide for tethering at the nanoparticle surface. Such terminal functional groups include azide, alkyne, aryl ester, amide, amine, aryl amide, aldehyde, acetyl, substituted aryl ester, alkyl ester, alkyl ketone, aryl ketone, substituted aryl ketone, ketone, alkyl halide, amnioxy, alcohols, aza-ylide, carboxylic acid, ester, amide, bicyclononyne, dihydrazide, halo-carbonyl, halosulfonyl, hydrazide, N-hydroxysuccinimide, norbomene, oxanorbomadiene, succinimidyl ester, isothiocyanate, iodoacetamide, monofluorinated and difluorinated cyclooctynes, maleimide, methylcyclopropene, isocyanopropanoate, hydrazine, nitrile, nitro, phosphine, phosphazide, tertazine, methyl-tetrazine, trans-cyclooctene, strained alkynes, dibenzocyclooctyne, biarylazacyclooctynone, propargyl, isocyanide, azadibenzylcyclooctyne, vinyl, sulfonyl ester, thioester, thiocarboxylate, thioester a sulfonyl halide, thiol, and thiolene. Such conjugation sites provide a position for linkage of the sialic-acid ligands to the surface of the nanoparticle through performance of click chemistry reactions. The linkage of the functional group on the nanoparticle (e.g., alkyne) is typically via the conjugate click chemistry via conjugate click chemistry functional group (e.g. azide) C2 carbon linkage,

The functional group of the sialic-acid ligand may be found at different positions on the sialic-acid core molecular structure located at the C1, C2, C4, C5, C7, C8, or C9 position. Accordingly, linkage of the sialic-acid ligand to surface of the nanoparticle may occur via conjugation at the C1, C2, C4, C5, C7, C8, or C9 position, yielding different orientations of the ligand in 3-dimensional space on the nanoparticle surface, which can influence ligand presentation to the immune cell of interest. Ligand presentation can, thus, be controlled in this manner to elicit the desired cell response upon contacting an immune cell via receptor binding.

Sialic-acid oligomers and sialic-acid polymers, and sialic-acid analogs thereof, with known spacing and/or density of ligands, are to be presented on the surface of the nanoparticles as ligands for Siglec receptors. By tethering specific sialic-acid analog ligands to the surface of the nanoparticles, the nanoparticles can contact a known set of immune cells expressing Siglec receptors in order to elicit specific biological responses thereby modulating inflammation. The diversity of the sialic-acid composition, structure, density, and architecture presented on the surface of the nanoparticle provides a means for regulating the degree and direction of the modulation of the response of immune cells.

The plurality of sialic-acids and/or sialic-acid analogs, in the form of monomers, polymers or oligomers, and with adjoining glycans, can be tethered to the surface of the nanoparticle by means of chemical conjugation. Such chemical conjugation can include, for example, click chemistry, carbodiimide chemistry, reductive amination, or chemisorption.

In a preferred embodiment, click chemistry reactions are employed for linkage of the sialic-acid ligands to the nanoparticle surface. Such click chemistry reactions are characterized as a class of biocompatible small molecule reactions commonly used for bioconjugation, which is employed in chemical ligation to modify other molecules, biomolecules, nanoparticles, and other surfaces. In general, click chemistry reactions possess the following properties: modularity, insensitivity to solvent parameters, high chemical yields, insensitivity towards oxygen and water, regiospecificity and stereospecificity, and a large thermodynamic driving force (>20 kcal/mol) to favor a reaction with a single reaction product. Click chemistry reactions provide a high reaction specificity giving control of both regio- and stereo-specificity. The reaction specificity is of particular usefulness for achieving the desired presentation of the sialic-acid ligands on the surface of the nanoparticle, thereby permitting optimal binding of the nanoparticle to immune cell Siglec receptors. The bonds formed by the click reactions during conjugation provide accessibility to highly stable covalent bonds between the sialic-acid ligand and the nanoparticle, which do not undergo rearrangement or reaction or result in degradation or hydrolysis in biological conditions.

A variety of different click chemistry reactions may be used to link the sialic-acid ligand to the surface of the nanoparticle. The use of such click chemistry reactions provides a controlled reaction medium for generation of nanoparticles with desired sialic-acid ligand density and spatial arrangement. The density and spatial arrangements of the sialic-acid ligands on the nanoparticle surface can be controlled, for example, by controlling the amounts of polymer with functional groups used forming the nanoparticles, the polymer molecular weight, polymer density, the number of functional groups per polymer, solvent, types of functional groups, concentration of ligands, types of click chemistry employed and type of click chemistry conjugate pairs. The spatial arrangements of the ligands can also be controlled by the linkage of the sialic-acids and other carbohydrates on the ligands. The length of the oligomer and/or polymer ligands can also control the density and spatial arrangement.

In various embodiments, an average molecular weight of a polymer, e.g., PEG, PLGA, PEG-PLGA block copolymer, or polysialic acid, can be determined by any of the methods known in the art, such as anion-exchange chromatography, gel permeation chromatography, viscosity measurements, among others.

Click chemistry reactions used for tethering of the sialic-acid ligand to the polymer are well known to those of skill in the art and include, for example, Huisgen 1,3-dipolar cycloaddition, copper(I)-catalyzed azide-alkyne cycloaddition (CuAAC) yielding 1,3-substituted products, ruthenium-catalyzed alkyne-azide cycloaddition (RuAAC) yielding 1,5-substituted triazoles, strain promoted alkyne azide cycloaddition (SPAAC) yielding 1,4-substituted products, strain-promoted alkyne-nitrone cycloaddition, alkene and tetrazine inverse-demand Diels-Alder, tetrazine trans-cyclooctene ligation, thiol-ene reaction, thiol-yne reaction, Staudinger reaction, [4+1] cycloaddition, quadricyclane ligation [2+2+2] cycloaddition, norbomene cycloaddition, and alkene tetrazole photoclick reaction.

The tethering of the sialic acids, or sialic-acid analogs (in the form of monomers, polymers, or oligomers), to the surface of the nanoparticles is performed in such a way so as to provide presentation of the sialic-acid ligand for maximum binding affinity to the Siglec receptors expressed on the surface of immune cells or sialic-acid ligand receptor expressed on the surface of viral particles. The ligand density can be controlled to provide the desired multivalent or polyvalent ligand interactions with the Siglec receptors when contacting the immune cells, as such interactions are correlated with a desired cellular immune response. Multivalent or polyvalent sialic-acid-receptor interactions may be controlled based upon the density of the ligands provided on the nanoparticle surface, and this density can influence the response elicited by the immune cells upon contact.

Suitably, the sialic acid or analog thereof may be immobilized on the surface of the nanoparticle. The sialic acid may be bound directly to the nanoparticle or via a linker such as polyethylene glycol. The nanoparticle may be derivatized or activated to allow binding of the sialic acid or analog. Alternatively, the nanoparticle may be derivatized or activated to allow binding of a linker to a nanoparticle and the linker may be attached to sialic acid. By linking the sialic acid or an analog thereof to a nanoparticle, the nanoparticle can be adapted to target a cell comprising a Siglec receptor to induce binding of the Siglec receptor such that production of pro-inflammatory cytokines within the cell is inhibited or production of anti-inflammatory cytokines is increased, thereby suppressing a pro-inflammatory immune response.

As disclosed, the use of click chemistry reactions requires that both polymers of the nanoparticle and the sialic-acid ligand be functionalized to permit the desired conjugation of the ligand to the nanoparticle surface. In one embodiment, the terminal alkyne is presented on the nanoparticle surface for covalent chemical conjugation with an azide-presenting sialic-acid ligand through performance of a copper(I)—catalyzed azide—alkyne (CuAAC) reaction; a copper-free reaction; a strain-promoted azide-alkyne (SPAAC) reaction; a tetrazine-alkene ligation reaction, or a trans-cyclooctene (TCO)-tetrazine reaction. The azide functional group can be added to the sialic-acid ligand using for example, sialyltransferase ST8SIA4.

In one aspect, Ligand/Nanoparticle conjugate pairs can be prepared with copper(I) azide-alkyne cycloaddition with sialic-acid ligands with azide and nanoparticles with alkyne functional groups, or with sialic-acid ligands with alkynes and nanoparticles with azide functional groups. Sialic-acid ligands with dibenzylcyclooctyne, difluorooctyne, or biarylazacyclooctynone can be reacted with azides via SPACC. Ligands with trans-cyclooctene can be reacted with nanoparticles having tetrazine functional groups.

In a specific non-limiting embodiment, PLGA-PEG-alkyne or PLGA-PEG-carboxylic acid nanoparticles are formed and then modified by covalently tethering a a2-8 polymer sialic-acid-azide ligand to the surface of the nanoparticle using click chemistry. Alkyne-functionalized PLGA nanoparticles are prepared via emulsion method based upon a protocol used by Greene et al. (Chem Sci 2018), yielding the core nanoparticle construct. The formed nanoparticle provides alkyne functional groups on the surface available for covalent chemical conjugation via click chemistry methods, including aCuAAC click chemistry reaction.

In a specific non-limiting embodiment, PLGA-PEG-DBCO or PLGA-PEG-carboxylic acid nanoparticles are formed and then modified by covalently tethering a a2-8 polymer sialic-acid-azide ligand to the surface of the nanoparticle using an SPACC click chemistry reaction. DBCO-functionalized PLGA nanoparticles are prepared via emulsion method based upon a protocol used by Greene et al. (Chem Sci 2018), yielding the core nanoparticle construct. The formed nanoparticle provides DBCO functional groups on the surface available for covalent chemical conjugation via click chemistry methods, including SPAAC click chemistry reactions.

In a specific non-limiting embodiment, PLGA-PEG-DBCO or PLGA-PEG-carboxylic acid nanoparticles are formed and then modified by covalently tethering a NeuAcα2-3Galβ1-4Glc-azide ligand to the surface of the nanoparticle using SPACC click chemistry reactions. DBCO-functionalized PLGA nanoparticles are prepared via emulsion method based upon a protocol used by Greene et al. (Chem Sci 2018), yielding the core nanoparticle construct. The formed nanoparticle provides DBCO functional groups on the surface available for covalent chemical conjugation via click chemistry methods, including SPAAC click chemistry reactions.

In a specific non-limiting embodiment, PLGA-PEG-DBCO or PLGA-PEG-carboxylic acid nanoparticles are formed and then modified by covalently tethering a NeuAcα2-6Galβ1-4Glc-azide ligand to the surface of the nanoparticle using Strain Promoted Azide-Alkyne Cycloaddition click chemistry. DBCO-functionalized PLGA nanoparticles are prepared via emulsion method based upon a protocol used by Greene et al. (Chem Sci 2018), yielding the core nanoparticle construct. The formed nanoparticle provides DBCO functional groups on the surface available for covalent chemical conjugation via click chemistry methods, including SPAAC click chemistry reactions.

The nanoparticles are prepared by mixing Poly(D,L-lactide-co-glycolide-COOH)-PEG-COOH (PLGA 10,000 Da-PEG-COOH 5000 Da) and Poly(lactide-co-glycolide)-b-Poly(ethylene glycol)-Azide (PLGA-PEG-alkyne, 10,000 Da PLGA:1,000 PEG Da) at a 75:25 (w/w) ratio of PLGA-COOH:PLGA-alkyne (DBCO). This 75:25 ratio is one embodiment of the density of azide functional groups on the nanoparticle surface. Other ratios of PLGA-PEG-COOH to PLGA-PEG-Alkyne (DBCO) used for nanoparticle preparation are 95:5, 90:10, 85:15, 80:20, 70:30, 65:35, 60:40, 55:45, 50:50. The ratio of PLGA-PEG-COOH to PLGA-PEG-Alkyne (DBCO) is designed to provide sufficient space between the functional groups to permit efficient conjugation of the polymer ligands and allow for the desired ligand density to be achieved.

Nanoparticles can be prepared comprising one or more polymers possessing different click chemistry functional groups for pairing with their sialic-acid ligand conjugation partner thereby allowing for the presentation of one or more types of sialic-acid ligands, with different densities and/or spatial arrangement, on the nanoparticle surface. Use of different conjugate pairs can be designed into the nanoparticle by employing one or more nanoparticle polymer/ligand pair.

In specific non-limiting embodiment, PLGA-PEG-DBCO//PLGA-PEG-Carboxylic acid nanoparticles are formed. A subsequent two-step process reaction is then used to conjugate two different ligands to the surface of the nanoparticle using heterogenous click chemistry. In the first step then modified by covalently tethering a NeuAcα2-3Galβ1-4GlcNAc-azide ligand to the surface of the nanoparticle using Strain Promoted Azide-Alkyne Cycloaddition click chemistry. In the second step reaction step, after washing of the nanoparticles to remove impurities, an a2-8 oligomeric sialic-acid-azide can be conjugated to the surface using CuAAC click chemistry. DBCO-/alkyne-functionalized PLGA nanoparticles are prepared via emulsion method based upon a protocol used by Greene et al. (Chem Sci 2018), yielding the core nanoparticle construct. The formed nanoparticle provides DBCO and alkyne functional groups on the surface available for covalent chemical conjugation via click chemistry methods in step-wise fashion. SPAAC followed by CuAAC.

By having different click chemistry functional groups available on the nanoparticle surface, different types of sialic-acid ligands can be conjugated to the surface of the nanoparticles. The density of the different functional groups can be controlled by the ratio of the different polymers to one another, the concentration of the polymers, and the type of click chemistry conjugate pairs, the type of click chemistry reactions, and the size and shape of the sialic-acid ligand. The number of different ligands that can be presented on the surface can be determined by those skilled in the art. In a non-limiting embodiment, the number of different ligands present on the nanoparticle surface is in the range of 1 to 20. The number of different ligands include, for example, is 1, 2, 3, 4, 5, 6, 7, 8, 9, 10, 11, 12, 13, 14, 15, 16, 17, 18, 19, or 20. In another embodiment, the number of different ligands present on the nanoparticle surface is in the range of 2 to 20. The number of different ligands include, for example, is 2, 3, 4, 5, 6, 7, 8, 9, 10, 11, 12, 13, 14, 15, 16, 17, 18, 19, or 20. In another embodiment, the nanoparticle will comprise at least two different sialic-acid ligands. In another embodiment, the nanoparticle will comprise least three different sialic-acid ligands. In another embodiment, the nanoparticle will comprise least four different sialic-acid ligands. In another embodiment, the nanoparticle will comprise least five different sialic-acid ligands.

In general, the density of the functional groups on the nanoparticle surface dictates the maximum ligand density that can be tethered to the surface of the nanoparticle via covalent chemical conjugation via click chemistry. The ligand density can be controlled and quantified in terms of the number of functional groups per square nanometer of surface area. The density reached allows for the transition of the polymer sialic-acid ligands to transition from mushroom confirmation to brush confirmation. The brush confirmation provides for the highest density packing of the polymer polysialic-acid. Tuning the density of the ligands on the nanoparticle surface provides a means by which the biological response of the target immune cells contacted by the nanoparticles can be modulated. The density of ligands presented on the surface of the nanoparticles can be quantified as nmol of ligands per mg of the total nanoparticle solids. The density can range from 0.05 nmol/mg to 50nmol/mg of the nanoparticles. The diameter of the nanoparticles can range from 25 nm to 200 nm.

The density of the ligands on the nanoparticle surface can be controlled by several methods including chemical conjugation techniques, ligand density on the polymer, ligand type, solvent, pH, and ionic strength. Ligand density on the surface of the nanoparticles can be tuned such that contacting immune cells with such nanoparticles results in an immune-modulating response, including an anti-inflammatory biological response. Control of the ligand density can also be used to modulate the magnitude of the desired anti-inflammatory response.

In some embodiments, the sialic acid or analogs thereof can be presented on the nanoparticle in groups of at least 2, at least 5, at least 10, at least 15, at least 20 or at least 25, at least 50, at least 100, at least 200, or at least 400. In some embodiments, the sialic acid or analogs thereof can be spaced on the surface of the nanoparticle such that they or the nanoparticle can bind to more than one Siglec receptor. In some embodiments, the sialic acid or analogs thereof can be spaced on the surface of the nanoparticle such that they or the nanoparticle can bind to multiple Siglec receptors presented on individual cell types, which may vary in the quantity of Siglec receptors presented on their plasma membrane.

In some embodiments the nanoparticle can comprise a polymer that includes sialic acid at a concentration in the range 0.05 nmol/mg of sialic acid to nanoparticles to 250 nmol/mg of sialic-acid to nanoparticles, preferably 0.5 nmol/mg to 25 nmol/mg, and most preferably 0.5 to 15 nmol of sialic-acid per mg of nanoparticle. In embodiments, a device can be coated with such a nanoparticle. In alternative embodiments a device can be formed from a polymer, for example wherein the device is a microparticle or nanoparticle, wherein sialic-acid is provided in the polymer at a concentration in the range 0.05 nmol/mg of sialic-acid to nanoparticle to 250 nmol/mg of sialic-acid to nanoparticle, preferably 1 nmol/mg to 25 μg/mg, and most preferably 2 to 15 nmol of sialic-acid per mg of nanoparticle.

In some embodiments, a nanoparticle can have a greatest cross-sectional width or diameter of less than about 1000 nm, less than about 500 nm, less than about 250 nm or less than about 200 nm. In embodiments, a nanoparticle can have a width greater than about 1 nm, greater than about 10 nm, greater than about 50 nm, or greater than about 100 nm. In embodiments, a nanoparticle coated with sialic-acid or a sialic-acid analog can have a greatest cross-sectional width or diameter in the range of about 130 nm to about 170 nm, more preferably a width of about 150 nm. In embodiments these range of sizes can be average widths of nanoparticles. In embodiments, at least 80% of the nanoparticles live within a disclosed range.

Suitably, in some embodiments around at least 80%, more preferably at least 90% of the particles have a greatest cross-sectional width between 130 nm to 170 nm. In embodiments, the particles can have an average greatest cross-sectional width of 150 nm with the particles having no width greater or less than a value not within one standard deviation of 150 nm. In some embodiments, the nanoparticle can have a volume equal to that of a sphere with a diameter between 10 nm to 500 nm, suitably between 50 nm to 250 nm, or 100 nm to 200 nm, or 130 nm to 170 nm.

In a preferred, non-limiting embodiment, the nanoparticle can have a volume equal to that of a sphere with a diameter of about 100 nm.

In another aspect of the invention, the linkage of the nanoparticles with sialic-acid ligands provides a means for the nanoparticles to evade the immune system, i.e., opsonization and phagocytosis via the reticuloendothelial system (RES). It is known that PEGylation of nanoparticles, i.e., the coating of nanoparticles with polyethylene glycol, provides barrier of protection from detection by immune cells. However, PEG has disadvantages of toxicity, immunogenicity, reduced cellular uptake, reduced binding, and nonbiodegradable or bioresorbable properties. Sialic acid coating of nanoparticles overcomes the disadvantages of PEG, and provides for a natural, non-immunogenic nanoparticle coating that can evade the RES and immune detection. Therefore, the nanoparticles disclosed herein possess the ability to evade immune detection and mitigate immunogenic response.

The nanoparticles, or microparticles, disclosed herein may further comprise a bioactive agent encapsulated within, adhered to the surface of, or integrated into the structure of said nanoparticles. For example, the nanoparticle can further comprise at least one of an antibiotic, an anti-viral agent, an anti-inflammatory, a cytokine, a cytokine inhibitor, an immunomodulator, an immunotoxin, an anti-angiogenic agent, an anti-hypertensive agent, an anti-edema agent, a radiosensitizer, an oligonucleotide comprising DNA or RNA, a peptide, an anti-cancer agent, or any combination thereof. Methods of preparing nanoparticles that include a bioactive agent encapsulated within, adhered to a surface of, or integrated into the structure of the nanoparticle are known to those skilled in the art.

The present disclosure further provides pharmaceutical or veterinary compositions comprising the sialic-acid ligand linked nanoparticles disclosed herein. Such a pharmaceutical composition is formulated to be compatible with its intended route of administration. Examples of routes of administration include both parenteral and non-parenteral administration methods including, for example, intravenous, intravitreal, oral, intraocular, subretinal, subtenons, intrascleral, periocular, intravenous, inhalational nasal and oral, intramuscular, intra-material, intraspinal, intrathecal, intracranial, intradermal, transdermal (topical), transmucosal, subcutaneous, pulmonary lavage, gastric lavage, intrahepatic, subcutaneous, and rectal administration.

Suitably, in some embodiments nanoparticles can be parenterally administered. After parenteral administration, nanoparticles can selectively accumulate in particular tissues or body locations. In some embodiments, nanoparticles can deliver a therapeutic payload to the cell or tissue. In some embodiments, nanoparticles can access diseased tissue through an enhanced permeability and retention effect.

In general, pharmaceutical compositions are provided comprising an effective amount of a nanoparticle with pharmaceutically acceptable diluents, preservatives, solubilizers, emulsifiers, adjuvants, and/or carriers. Such compositions include diluents of various buffer content (e.g., Tris-HCl, acetate, phosphate), pH and ionic strength; additives such as detergents and solubilizing agents (e.g., tween 80, polysorbate 80), anti-oxidants (e.g., ascorbic acid, sodium metabisulfite), preservatives (e.g., thimerosal, benzyl alcohol) and bulking substances (e.g., lactose, mannitol). Such compositions may influence the physical state, stability, rate of in vivo release, and rate of in vivo clearance of the nanoparticle. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be formulated into a dried powder, such as lyophilized form.

The present disclosure provides for a method of treating immune and inflammatory-related diseases, including, but not limited to, dry and wet macular degeneration, retinal vascular disease, diabetic retinopathy, diabetic macular edema, cystoid macular edema, proliferative diabetic retinopathy, proliferative vitreoretinopathy, dry eye, allergic conjunctivitis, rheumatoid arthritis, inflammatory arthritis, lupus, nephritis, immune complex nephropathy, allergic esophagitis, allergic gastritis, hepatitis, fibrotic diseases of the liver, idiopathic pulmonary fibrosis, acute respiratory distress syndrome, sepsis, bacterial and viral infections, influenza. SARS-CoV-1 and SARS-CoV-2, HIV/AIDS, Group B streptococcal infection, Neisseria infection, cancers involving solid organs, or hematopoetic cancers, in each case in an afflicted subject through the administration of such pharmaceutical compositions. The present disclosure provides a method of modulating an inflammatory response in a cell, the method comprising: providing sialic acid or analogs thereof to a cell, wherein the sialic acid or analogs are presented on a nanoparticle such that a pro-inflammatory response in a cell is suppressed or an anti-inflammatory response in increased in the cell. In embodiments, the method provides for the suppression of a pro-inflammatory response. In alternative embodiments, the method provides for the increase in an anti-inflammatory response. In some embodiments, the method provides for the enhancement of a pro-inflammatory response in situations such as infections, or cancer.

The term “treatment” or “treating” as used herein to characterize a method or process that is aimed at (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition: or (5) curing the disease, disorder, or condition. A treatment may be administered prior to the onset of the disease, disorder, or condition, for a prophylactic or preventive action. Alternatively, or additionally, the treatment may be administered after initiation of the disease, disorder, or condition, for a therapeutic action.

Depending on the route of administration and disease, effective doses may be calculated according to the body weight, body surface area, primary organ/tumor size, and/or number, sizes, and/or types of metastases of the subject to be treated. Optimization of the appropriate dosages can readily be made by one skilled in the art considering pharmacokinetic data observed in human clinical trials. The final dosage regimen will be determined by considering various factors which modify the action of the drugs, e.g., the drug's specific activity, the severity of the damage and the responsiveness of the patient, the age, condition, body weight, sex and diet of the patient, the severity of any present infection, time of administration, the use (or not) of other therapies, and other clinical factors.

In one aspect, the pro-inflammatory response can be suppressed by at least 10%, at least 20%, at least 30%, at least 40%, at least 50%, at least 60%, at least 70%, at least 80%, at least 90%, or at least 99%. In alternative embodiments, the anti-inflammatory response can be increased by at least 10%, at least 20%, at least 30%, at least 40%, and at least 50%, at least 60%, at least 70%, at least 80%, at least 90%, at least 99%. In other embodiments the compositions disclosed herein can provide for the suppression of a pro-inflammatory response and an increase in an anti-inflammatory response.

Suitably pro-inflammatory cytokines can be measured to determine the efficacy of nanoparticle drug treatment. Such measurements can be made during the actual treatment of a subject, or alternatively, during animal testing of the nanoparticles disclosed herein. In embodiments, pro-inflammatory cytokines can include, for example, TNF-α and IL-6. Suitably anti-inflammatory cytokines can also be measured, for example IL-10. The skilled person would know of suitable assay methods to measure such cytokines. For example, the Bio-Plex™ Cytokine Assay (Bio-Rad) may be used. To determine whether a cell produces greater or less proinflammatory cytokines, a suitable method which can be used is that cells are resuspended and seeded at 2×10⁵ cells/ml and 200 μl per well in a 96 well plate. They can then be left to adhere to the plate overnight and be treated with LPS and ligands for 24 hours at range of concentrations. Supernatant can then be removed and stored at −70° C. Cytokine levels can then be assessed by ELISA (R&D systems). As will be appreciated a similar method can be applied to determine anti-inflammatory cytokines.

In an embodiment, TNF-α levels can be suitably determined by coating a 96 well plate with TNF-α capture antibody diluted in 1× phosphate buffered saline (PBS) overnight. All steps can be carried out at room temperature. The wells can be washed three times in 1×PBS/0.1% Polyoxyethylene sorbitan monolaurate (Tween 20) before being blocked for one hour with 1% BSA (BDH) dissolved in 1×PBS. The washing step can be repeated and 50 μl of treated cell supernatants or standards ranging from 2000 μg/ml to 0 μg/ml can be added to the wells and left for 2 hours. Subsequently supernatant can be aspirated out, the wells washed 3 times and 50 μl of TNF-α detection antibody diluted in 1% BSA/1×PBS can be added for 2 hours. Again, wells can be washed three times and Horse Radish Peroxidase (HRP) conjugated antibody can be added at 1 in 200 dilution in 1% BSA/1×PBS for 20 minutes. At this stage, the plate can be covered in aluminum foil. Once wells have been washed 3,3′,5,5′-tetramethylbenzidine (TMB) can be added for 20 minutes and again protected from light. 1M hydrochloric acid can be added to halt the reaction and absorbance read on a plate reader at 450 nM. TNF-α concentrations can then be extrapolated from the standard curve. As will be appreciated, a similar methodology can be applied to determine other cytokine levels, substituting the TNF-α detection antibody for a detection antibody or other agent specific for the applicable cytokine.

In an embodiment, IL-10 levels can be suitably determined by coating a 96 well plate with IL-10 capture antibody diluted in 1× phosphate buffered saline (PBS) overnight. All steps can be carried out at room temperature. The wells can be washed three times in 1×PBS/0.1% Polyoxyethylene sorbitan monolaurate (Tween 20) before being blocked for one hour with 1% BSA (BDH) dissolved in 1×PBS. The washing step can be repeated and 50 μl of treated cell supernatants or standards ranging from 2000 μg/ml to 0 μg/ml can be added to the wells and left for 2 hours. Subsequently supernatant can be aspirated out, the wells washed 3 times and 50 μl of IL-10 detection antibody diluted in 1% BSA/1×PBS can be added for 2 hours. Again, wells can be washed three times and Horse Radish Peroxidase (HRP) conjugated antibody can be added at 1 in 200 dilution in 1% BSA/1×PBS for 20 minutes. At this stage, the plate can be covered in aluminum foil. Once wells have been washed 3,3′,5,5′-tetramethylbenzidine (TMB) can be added for 20 minutes and again protected from light. 1M hydrochloric acid can be added to halt the reaction and absorbance read on a plate reader at 450 nM. IL-10 concentrations can then be extrapolated from the standard curve. As will be appreciated, a similar methodology can be applied to determine other cytokine levels, substituting the IL-10 detection antibody for a detection antibody or other agent specific for he applicable cytokine.

To determine whether the treated subject or test animal produces a greater or lesser pro-inflammatory response, a method that may be used is analysis of serum cytokine levels. For example, this may be achieved by the collection of 50 μl blood from the treated subject using a capillary tube. This blood is allowed to clot at room temperature for 30 minutes prior to centrifugation at 1300 rpm to pellet red blood cells. Serum is decanted to a clean micro-centrifuge tube and analyzed by ELISA. For more extensive analysis, a larger volume of blood (approximately 600 μl-1 ml) may be taken by direct cardiac puncture, thus allowing for a greater volume of serum to be collected and analyzed by ELISA or such other technique. Other suitable techniques to determine whether the treated subject or test animal produces a greater or lesser pro-inflammatory response will be known in the art, particularly to detect and measure cytokines.

In some embodiments of the method, wherein the nanoparticle is provided, it has been determined that binding to a Siglec receptor on a cell can inhibit production of pro-inflammatory cytokines by the cell and induce anti-inflammatory cytokines. In embodiments, the binding of a nanoparticle to a Siglec receptor on a cell can result in activation of the receptor and can induce internalization of the receptor and the nanoparticle into the cell. Production of pro-inflammatory cytokines by the cell can be inhibited and/or the production of anti-inflammatory cytokines can be increased following internalization of a nanoparticle.

Accordingly, a method of treating an inflammatory disease, in a subject in need thereof, is provided, said method comprising administering to a subject sialic-acid or an analog thereof, wherein the sialic acid or the analog is presented on a nanoparticle such that a pro-inflammatory immune response is suppressed or an anti-inflammatory immune response is increased in the subject.

Said method may comprise: identifying a subject having a pro-inflammatory immune response and/or suffering from a disorder associated with or caused by a pro-inflammatory immune response or at risk of developing a pro-inflammatory immune response or a disorder associated with or caused by a pro-inflammatory immune response, administering to a subject sialic-acid or analogs thereof, wherein the sialic-acid or analogs are presented on a nanoparticle.

In specific embodiments, the method can be used to treat a subject with pulmonary disease, including inflammatory and non-inflammatory conditions of the lung, but not exclusive to tuberculosis, chronic obstructive pulmonary disorder (COPD), asthma, acute lung injury, acute respiratory distress syndrome, cystic fibrosis, bronchiectasis, pulmonary fibrosis interstitial lung disease, pulmonary vascular disease, influenza, viral pneumonia, bacterial pneumonia, allergic bronchitis, nonallergic bronchitis, rhinitis, and fibrosing alveolitis.

In some embodiments, the method can be used for the treatment of rheumatic diseases including but not exclusive to rheumatoid arthritis, fibromyalgia, systemic lupus erythematosus, systemic sclerosis (scleroderma), psoriatic arthritis, ankylosing spondylitis, sjogrens syndrome, polymyalgia rehumatica, gout, osteoarthritis, infectious arthritis, and juvenile idiopathic arthritis.

In some embodiments, the method can be used for the treatment of gastrointestinal inflammation including but not exclusive to Crohn's disease, ulcerative colitis, irritable bowel syndrome, celiac disease, diverticulitis, gastroesophageal reflux, lactose intolerance, peptic ulcer, cholecystitis, gastritis, colitis, pancreatitis, autoimmune hepatitis, hepatitis, infectious hepatitis, and pancreatitis.

In some embodiments, the method can be used for the treatment of cardiovascular diseases including but not exclusive to septic shock, atherosclerosis, diastolic dysfunction, heart failure, cardiac fibrosis, coxsackie myocarditis, congenital heart block, autoimmune myocarditis, giant cell myocarditis, and inflammation.

In some embodiments, the method can be used for the treatment of renal inflammation including but not exclusive to kidney transplant rejection, glomerulonephritis, acute nephritis, cystitis, prostatitis diabetic nephritis, diabetic kidney disease, and urinary tract infections.

In some embodiments, the method can be used for the treatment of dermatologic inflammation including but not exclusive to dermatitis, eczema, inflammatory rashes, scleroderma, keloid, acne, sarcoidosis, tinea cruris, tinea corporis, tinea pedis, tinea capitis, tinea unguium, rosacea, vitiligo, lichen sclerosis, autoimmune urticaria, dermatomyositis, and hidradenitis suppurativa.

In some embodiments, the method can be used for the treatment of neurological inflammation including but not exclusive to neuromyelitis, multiple sclerosis, encephalitis, neuro sarcoid, Alzheimers, amyotrophic lateral sclerosis, and huntingtons chorea

In some embodiments, the method can be used for the treatment of autoimmune inflammation including but not exclusive to diabetes, SLE, multiple sclerosis, sjogrens syndrome. Addison's disease, Graves Disease, Hashimotos thyroiditis, myasthenia gravis, autoimmune vasculitis, celiac disease, pernicious anemia, alopecia areata, autoimmune hepatitis, autoimmune angioedema, autoimmune encephalomyelitis, autoimmune inner ear disease, Guillain barre. Kawasaki disease, lambert-eaton syndrome, Vogt-Koyanagi-Harada Syndrome, systemic vasculitis, giant cell arteritis, sarcoidosis, and polyarteritis nodosa.

In some embodiments, the method can be used for the treatment of viral inflammation including but not exclusive to influenza A, B, C, SARS-CoV1, SARS-CoV2, Newcastle Disease, Sendai virus, Polyomavirus, HIV, Flavivirus, Caclivirus, Herpes virus, Picoronovirus, and Coronavirus.

In some embodiments, the method can be used for the treatment of fungal inflammation including but not exclusive to fungemia, fungal abscess, fungal keratitis, candidiasis, tinea pedis, and tinea cruris.

In some embodiments, the method can be used for the treatment of parasitic inflammation including but not exclusive to amoebiasis, giardiasis, toxoplasmosis, and toxocara.

In some embodiments, the method can be used for the treatment of fibrotic disease including but not exclusive to idiopathic pulmonary fibrosis, myelofibrosis, hepatic fibrosis, cardiac fibrosis with dystolic dysfunction and CHF, kidney fibrosis, retinal fibrosis, dermal fibrosis, and scarring,

In some embodiments, the method can be used for the treatment of acute life threatening inflammation including but not exclusive to sepsis and cytokine storm sialic-acid In a specific embodiment, provided are methods of treating a plurality of ocular inflammatory diseases such as macular degeneration, uveitis, optic neuritis, neuromyelitis, and inflammation arising from infections of the eye, eye exposure to drugs and toxins, and general immune disorders including autoimmune disorders. In a non-limiting embodiment, provides useful methods are provided for preventing, treating, or ameliorating a macular degeneration such as dry macular degeneration, wet macular degeneration, geographic atrophy, intermediate macular degeneration and age-related macular degeneration in a patient. The methods of treating, preventing or ameliorating ocular inflammation, including macular degeneration, comprise administering a composition of sialic-acid ligand nanoparticles to a patient suffering from, or a risk of developing, ocular inflammation such as macular degeneration.

In some embodiments, an ophthalmic preparation is provided as an eye drop, an eye ointment or an ophthalmic injection. For an ophthalmic injection, intravitreous or subconjunctival injection, may be used to administer the nanoparticles.

Co-administration of additional compounds having applications in methods to treat, prevent or ameliorate a macular degeneration may be co-administered in conjunction with the nanoparticle containing pharmaceutical compositions used for treating macular degeneration. For example, anti-angiogenic pharmaceuticals for the treatment of wet age-related macular degeneration such as pegaptanib sodium, ranibizumab, bevacizumab, aflibercept and brolucizumab can be used as a combination. While particular embodiments of the present disclosure have been shown and described, it will be obvious to those skilled in the art that changes and modifications can be made without departing from the disclosure in its broader aspects. Therefore, the appended claims are to encompass within their scope all such changes and modifications as fall within the true spirit and scope of this disclosure.

Accordingly, in a first example embodiment, the present invention is a particle, comprising a molecule represented by the following structural formula:

P-L-G,

In a 1^{st} aspect of the 1^{st} example embodiment, P is a biocompatible polymer scaffold comprising at least one biocompatible polymer selected from the group consisting of polyglycolic acid, poly(L-lactic acid), poly(lactic-co-glycolic acid), polycaprolactone, poly(3-hydroxybutyric acid), poly(ethylene glycol), polyethylene oxide, Pluronic F127, Pluronic F68, poloxamer, poly(hydroxymethylmethacrylate), polyvinyl alcohol, poly(vinylpyrrolidone), hyaluronic acid, heparin, heparin sulfate, sialic-acid and chitosan: G is a polysialic acid (PSA) comprising from 5 to 200 repeat units of sialic acid; and L is a covalent linker, or a pharmaceutically acceptable salt thereof.

In a 2^{nd} aspect of the 1^{st} example embodiment, the polymer scaffold comprises a block copolymer PLGA-PEG. The remainder of features and example features of the 1^{st} example embodiment are as they are defined with respect to its varkious aspects

In a 3^{rd} aspect of the 1^{st} example embodiment, P is represented by the following structural formula:

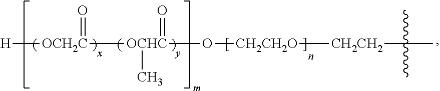

wherein the symbol [symbol] represents the point of attachment of the polymer to the linker L, and further wherein: x is an integer from 0 to 20, for example from 0 to 10, y is an integer from 0 to 20, for example from 0 to 10, m is an integer from 1 to 1000, for example from 1 to 500, n is an integer from 5 to 450, provided that x and y are not simultaneously 0. The remainder of features and example features of the 1st example embodiment are as they are defined with respect to its various aspects.

In a 4^{th} aspect of the 1^{st} example embodiment, nG is represented by any one of the following structural formulas:

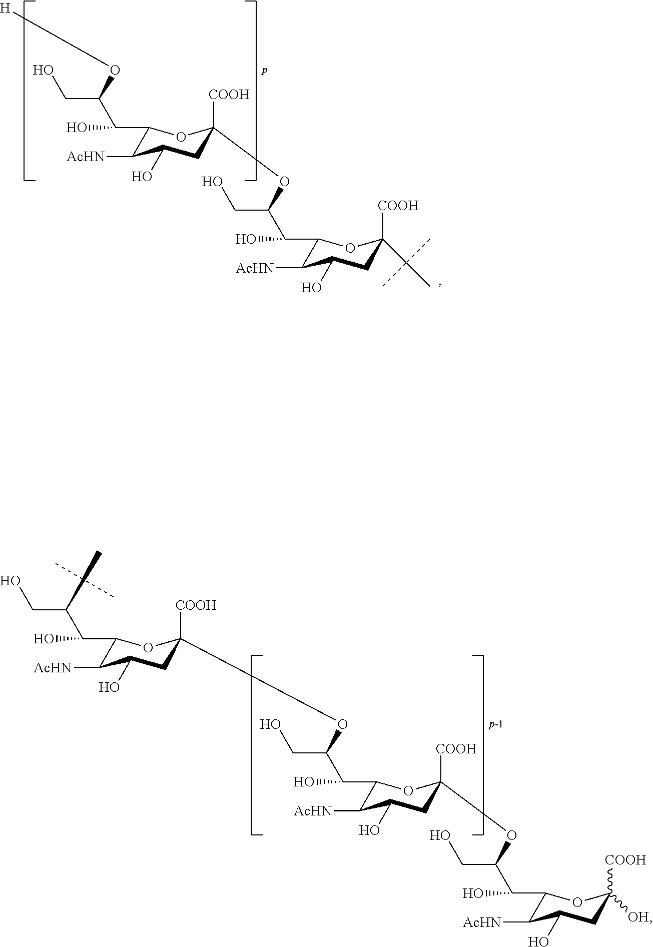

or a pharmaceutically acceptable salt thereof, wherein the symbol [symbol] represents the points of attachment of G to the linker L, and further wherein p, also known as the degree of polymerization (DP) is an integer from 4 to 198. The remainder of features and example features of the 1st example embodiment are as they are defined with respect to its various aspects.

In a 5^{th} aspect of the 1^{st} example embodiment the value of p is selected from any one of the following ranges: from 10 to 20, from 20 to 30, from 30 to 40, from 40 to 50, and from 50 to 60. For example, the value of p can be from 5 to 25 or from 10 to 20. For example, the value of p can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 43, 44, 45, 46, 47, 48, 49, 50, 51, 52, 53, 54, 55, 56, 57, 58, 59, 60. The remainder of features and example features of the 1^{st} example embodiment are as they are defined with respect to its various aspects.

In a 6^{th} aspect of the 1^{st} example embodiment, the linker L is represented by any one of the following structural formulas, wherein the symbol [symbol] represents the point of attachment of the linker L to G, and the symbol [symbol] represents the point of attachment of the linker L to P:

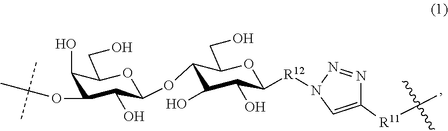

wherein: R¹¹ is —C(O)NH— or —CH₂—NH—C(O)—CH₂—O—; and R¹² is absent or is any one of —O—(CH₂)₁₋₁₀—, —(O—CH₂CH₂)₁₋₁₀—, —N(X¹¹)—(CH₂)₁₋₁₀—, —N(X¹²)—O—(CH₂)₁₋₁₀—, or —NHNH—(CH₂)₁₋₁₀—, wherein X¹¹ is H or acetyl, and X¹² is H or methyl;

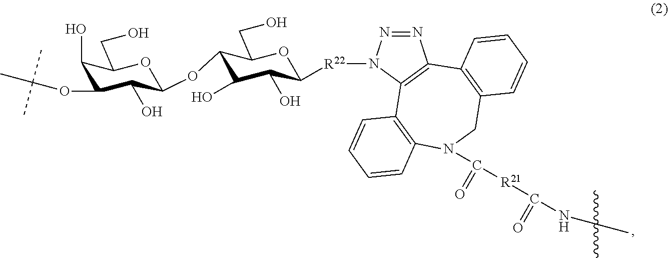

wherein: R²¹ is —(CH₂)₁₋₁₀— or —(CH₂CH₂—O)₁₋₁₀—(CH₂)₁₋₁₀—; and R² is absent or is any one of —O—(CH₂)₁₋₁₀—, —(O—CH₂CH₂)₁₋₁₀—, —N(X²¹)—(CH₂)₁₋₁₀—, —N(X²²)—O—(CH₂)₁₋₁₀—, or —NH—NH—(CH₂)₁₋₁₀—, wherein X²¹ is H or acetyl, and X²² is H or methyl;

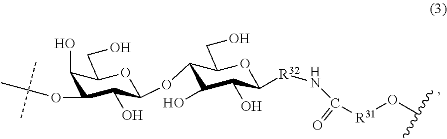

wherein: R³¹ is —(CH₂)₁₋₁₀—; and R³² is absent or is any one of —O—(CH₂)₁₋₁₀—, —(O—CH₂CH₂)₁₋₁₀—, —N(X¹¹)—(CH₂)₁₋₁₀—, —N(X¹²)—O—(CH₂)₁₋₁₀—, or —NHNH—(CH₂)₁₋₁₀—, wherein X³¹ is H or acetyl, and X³² is H or methyl;

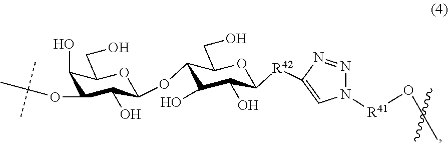

wherein: R⁴¹ is —(CH₂)₁₋₁₀—; and R⁴² is absent or is any one of —O—(CH₂)₁₋₁₀—, —(O—CH₂CH₂)₁₋₁₀—, —N(X⁴¹)—(CH₂)₁₋₁₀—, —N(X⁴²)—O—(CH₂)₁₋₁₀—, or —NHNH—(CH₂)₁₋₁₀—, wherein X⁴¹ is H or acetyl, and X⁴² is H or methyl;

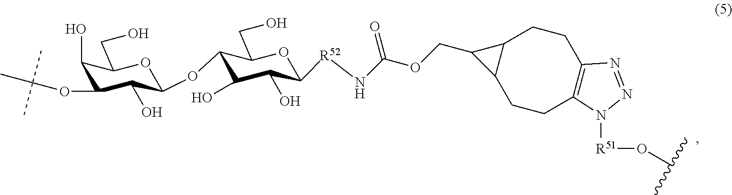

wherein: R⁵¹ is —(CH₂)₁₋₁₀—; and R⁵² is absent or is any one of —O—(CH₂)₁₋₁₀—, —(O—CH₂CH₂)₁₋₁₀—, —N(X⁵¹)—(CH₂)₁₋₁₀—, —N(X⁵¹)—O—(CH₂)₁₋₁₀—, or —NHNH—(CH₂)₁₋₁₀—, wherein X⁵¹ is H or acetyl, and X⁵² is H or methyl;

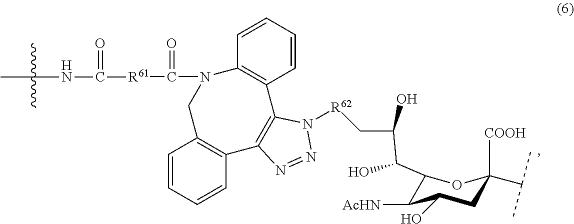

wherein: R⁶¹ is —(CH₂)₁₋₁₀— or —(CH₂CH₂—O)₁₋₁₀—(CH₂)₁₋₁₀—; R² absent or is —(CH₂)₁₋₁₀—O—(CH₂)₁₋₁₀—NH—;

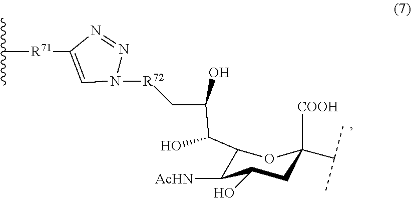

wherein: R⁷¹ is —NHC(O)— or —OCH₂—C(O)NH—CH₂—; and R⁷ is —(CH₂)₁₋₁₀—O—(CH₂)₁₋₁₀—NH—;

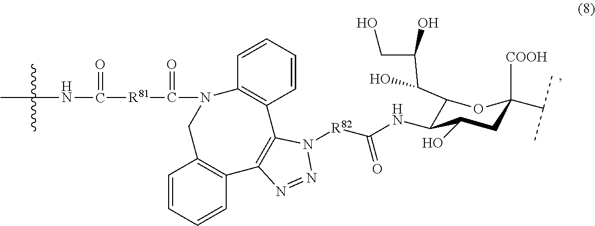

wherein: R⁸¹ and R⁸², each independently is —(CH₂)₁₋₁₀— or —(CH₂CH₂—O)₁₋₁₀—(CH₂)₁₋₁₀—;

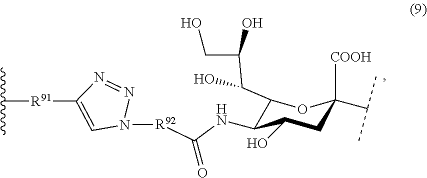

wherein: R⁹¹ is —NHC(O)— or —OCH₂—C(O)NH—CH₂—; and R⁹² is —(CH₂)₁₋₁₀— or —(CH₂CH₂—O)₁₋₁₀—(CH₂)₁₋₁₀—;

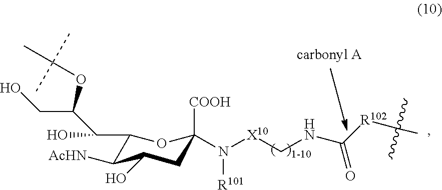

wherein: R¹⁰¹ is H or methyl; X¹⁰ is O or NH; and R¹⁰² is —CH₂O—, or a moiety represented by any one of the following structural formulas:

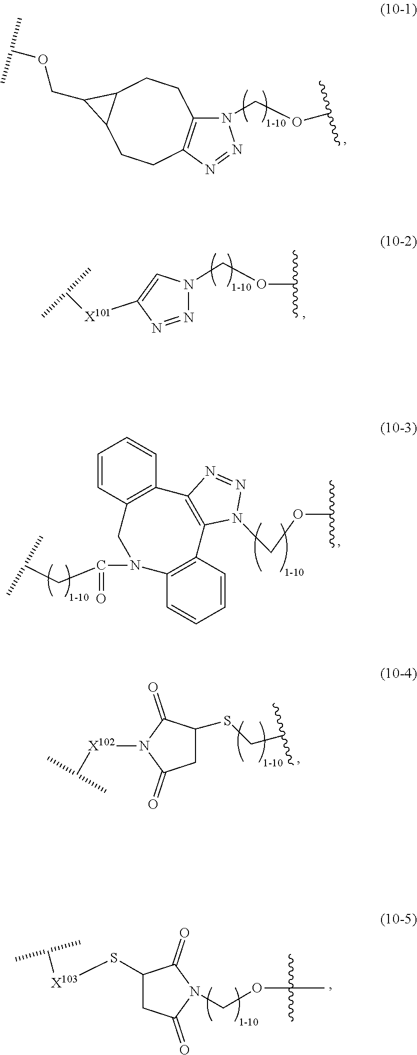

wherein X¹⁰¹, X¹⁰², and X¹⁰³, each independently is —(CH₂)₁₋₁₀— or —CH₂CH₂—(OCH₂CH₂)₁₋₁₀, and wherein the symbol [symbol] represents the point of attachment to the carbonyl A;

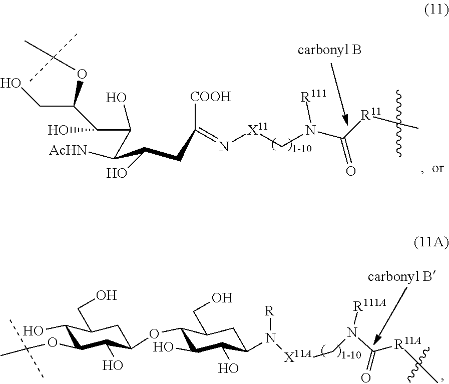

wherein: R¹¹¹ and R^{111A}, each independently, is H or a C1-C3 alkyl; X¹¹ and X^{11A}, each independently, is O or NH; and R¹¹ and R^{11A}, is independently, is —CH₂O—, or a moiety represented by any one of the following structural formulas:

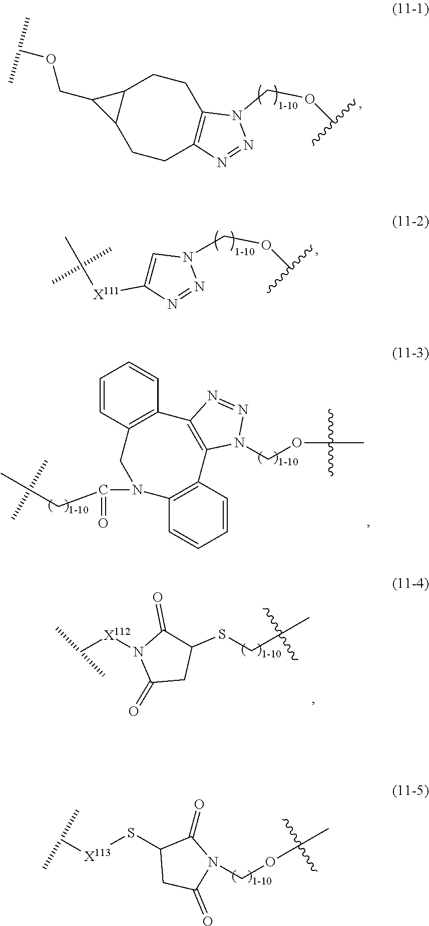

wherein X¹¹¹, X¹¹², and X¹¹³, each independently is —(CH₂)₁₋₁₀— or —CH₂CH₂—(OCH₂CH₂)₁₋₁₀, and wherein the symbol [symbol] represents the point of attachment to the carbonyl B or the carbonyl B′;

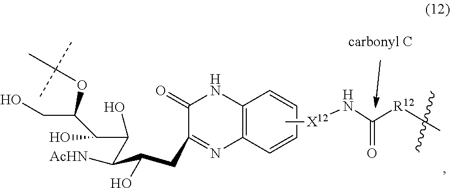

wherein: X¹² is —(CH₂)₁₋₁₀— or —C(O)—(CH₂)₁₋₁₀—; and R¹² is —CH₂O—, or a moiety represented by any one of the following structural formulas

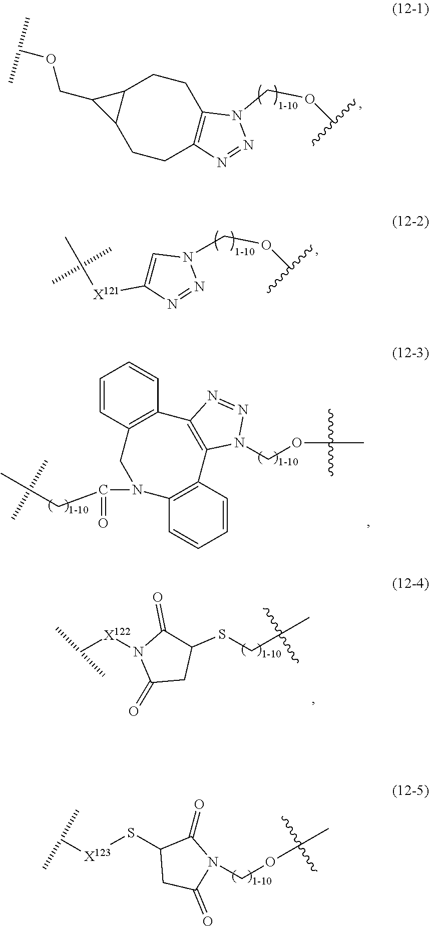

wherein X¹²¹, X¹²², and X¹²³, each independently is —(CH₂)₁₋₁₀— or —CH₂CH₂—(OCH₂CH₂)₁₋₁₀, and wherein the symbol [symbol] represents the point of attachment to the carbonyl C; and

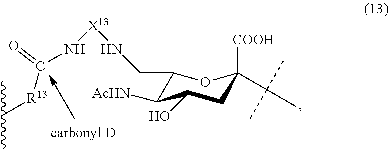

wherein: X¹¹ is -Ph- or —CH₂-Ph-CH₂—, wherein Ph is phenyl; and R¹³ is is —CH₂O—, or a moiety represented by any one of the following structural formulas:

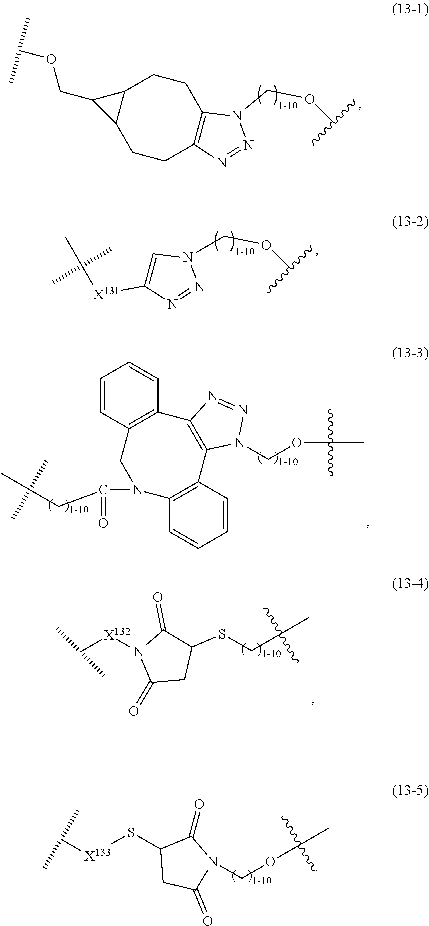

wherein X¹³¹, X¹³², and X¹³³, each independently is —(CH₂)₁₋₁₀— or —CH₂CH₂—(OCH₂CH₂)₁₋₁₀—, and wherein the symbol [symbol] represents the point of attachment to the carbonyl D:

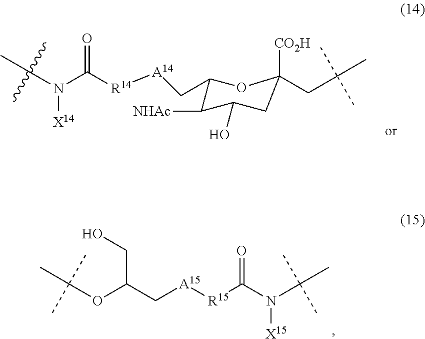

wherein: X¹⁴ and X¹⁵ each independently is H or methyl, A¹⁴ and A¹⁵ each independently is NR^{A}, NR^{A}NR^{B}, O or S, wherein R^{A} and R^{B} for each occurrence independently is selected from H, C1-4 alkyl and C6-C18 aryl; and R¹⁴ and R¹⁵ each independently is —(CH₂)₁₋₁₀— or —CH₂CH₂—(OCH₂CH₂)₁₋₁₀—. The remainder of features and example features of the 1^{st} example embodiment are as they are defined with respect to its various aspects.

In a 8^{th} aspect of the 1^{st} example embodiment, the linker L is represented by the following structural formula:

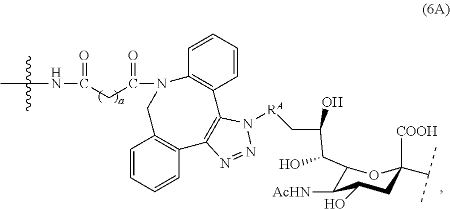

wherein: a is an integer from 2 to 6; and R^{A} is absent or is R^{A} is absent or is —(CH₂)₁₋₂—O—(CH₂)₁₋₂—NH—. The remainder of features and example features of the 1 example embodiment are as they are defined with respect to its various aspects.

In a 9^{th} aspect of the 1^{st} example embodiment, the P is a PLGA(10 k)-PEG(5 k). The remainder of features and example features of the 1^{st} example embodiment are as they are defined with respect to its various aspects.

In a 10 aspect of the 1^{st} example embodiment, the weight of G per unit weight of P (ligand density) is from 0.5 μg/mg to 100 μg/mg. For example, ligand density can be from 10 to 75 μg/mg or from 10 to 75 μg/mg. In other examples, ligand density can be 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mg. The remainder of features and example features of the 1^{st} example embodiment are as they are defined with respect to its various aspects.

In a 2^{nd} example embodiment, the present invention is a method of treating a subject suffering from an ophthalmic disease, comprising administering to the subject a therapeutically effective amount of a particle of any of the aspects of the 1^{st} example embodiment.

In a 1^{st} aspect of the 2^{nd} example embodiment, the ophthalmic disease is a dry age-related macular degeneration, a wet age-related macular degeneration, non-proliferative diabetic retinopathy, proliferative diabetic retinopathy, macular edema, uveitis, dry eyes, conjunctivitis, thyroid ophthalmopathy, endophthalmitis, retinal degeneration, glaucoma, retinal vein occlusions, blepharitis, keratitis, an ocular infection, or a cataract. The remainder of features and example features of the 2^{nd} example embodiment are as they are defined with respect to its various aspects.

In a 2^{nd} aspect of the 2^{nd} example embodiment, the sialic acid ligand is an agonist of one or more of Siglec 3, 5, 7, 8, 9, 10, 11, or 15, and antagonist of one or more of Siglec 14 or 16. The remainder of features and example features of the 2^{nd} example embodiment are as they are defined with respect to its various aspects.

In the 3^{rd} example embodiment, the present invention is a method of treating a subject suffering from an inflammatory disease, comprising: administering to the subject a therapeutically effective amount of a particle according to any aspect of the 1^{st} example embodiment.

In a 1^{st} aspect of the 3^{rd} example embodiment, the route of administration is one or more of intravenous, intravitreal, oral, intraocular, subretinal, subtenons, intrascleral, periocular, inhalational nasal and oral, intramuscular, intra-areterial, intraspinal, intrathecal, intracranial, intradermal, transdermal (topical), transmucosal, subcutaneous, pulmonary lavage, gastric lavage, and intrahepatic, subcutaneous, or rectal.

In a 2^{nd} aspect of the 3^{rd} example embodiment, the inflammatory disease is tuberculosis, chronic obstructive pulmonary disorder (COPD), asthma, acute lung injury, acute respiratory distress syndrome, cystic fibrosis, bronchiectasis, pulmonary fibrosis interstitial lung disease, pulmonary vascular disease, influenza, viral pneumonia, bacterial pneumonia, allergic bronchitis, nonallergic bronchitis, rhinitis, or fibrosing alveolitis. The remainder of features and example features of the 3^{rd} example embodiment are as they are defined with respect to its various aspects.

In a 3^{rd} aspect of the 3^{rd} example embodiment, the inflammatory disease is rheumatoid arthritis, fibromyalgia, systemic lupus erythematosus, systemic sclerosis (scleroderma), psoriatic arthritis, ankylosing spondylitis, sjogrens syndrome, polymyalgia rehumatica, gout, osteoarthritis, infectious arthritis or juvenile idiopathic arthritis. The remainder of features and example features of the 3^{rd} example embodiment are as they are defined with respect to its various aspects.

In a 4^{th} aspect of the 3^{rd} example embodiment, the inflammatory disease is Crohn's disease, ulcerative colitis, irritable bowel syndrome, celiac disease, diverticulitis, gastroesophageal reflux, lactose intolerance, peptic ulcer, cholecvstitis, gastritis, colitis, pancreatitis, autoimmune hepatitis, hepatitis, infectious hepatitis, or pancreatitis. The remainder of features and example features of the 3^{rd} example embodiment are as they are defined with respect to its various aspects.

In a 5^{th} aspect of the 3^{rd} example embodiment, the inflammatory disease is septic shock, atherosclerosis, diastolic dysfunction, heart failure, cardiac fibrosis, coxsackie myocarditis, congenital heart block, autoimmune myocarditis, or giant cell myocarditis. The remainder of features and example features of the 3^{rd} example embodiment are as they are defined with respect to its various aspects.

In a 6^{th} aspect of the 3^{rd} example embodiment, the inflammatory disease is kidney transplant rejection, glomerulonephritis, acute nephritis, cystitis, prostatitis, diabetic nephritis, or diabetic kidney disease. The remainder of features and example features of the 3^{rd} example embodiment are as they are defined with respect to its various aspects.

In a 7^{th} aspect of the 3^{rd} example embodiment, the inflammatory disease is dermatitis, eczema, inflammatory rashes, scleroderma, keloid, acne, sarcoidosis, tinea cruris, tinea corporis, tinea pedis, tinea capitis, tinea unguium, rosacea, vitiligo, lichen sclerosis, autoimmune urticaria, dermatomyositis, or hidradenitis suppurativa. The remainder of features and example features of the 3^{rd} example embodiment are as they are defined with respect to its various aspects.

In a 8^{th} aspect of the 3^{rd} example embodiment, the inflammatory disease is diabetes. SLE, multiple sclerosis, sjogrens syndrome, Addison's disease, Graves' Disease, Hashimotos thyroiditis, myasthenia gravis, autoimmune vasculitis, celiac disease, pernicious anemia, alopecia areata, autoimmune hepatitis, autoimmune angioedema, autoimmune encephalomyelitis, autoimmune inner ear disease, Guillain barre, Kawasaki disease, lambert-eaton syndrome. Vogt-Koyanagi-Harada Syndrome, systemic vasculitis, giant cell arteritis, sarcoidosis, or polyarteritis nodosa. The remainder of features and example features of the 3^{rd} example embodiment are as they are defined with respect to its various aspects.

In a 9^{th} aspect of the 3^{rd} example embodiment, the inflammatory disease is neuromyelitis, multiple sclerosis, encephalitis, neuro sarcoid. Alzheimer's, amyotrophic lateral sclerosis, or Huntington's chorea, transverse myelitis, Guillain barre syndrome, Parkinsons disease, or benign essential tremors. The remainder of features and example features of the 3^{rd} example embodiment are as they are defined with respect to its various aspects.

In a 10^{th} aspect of the 3^{rd} example embodiment, the inflammatory disease is a viral disease caused by influenza A, influenza B, influenza C, SARS-CoV1, SARS-CoV2, Newcastle Disease virus, Sendai virus, Polyomavirus, HIV, Flavivirus, Caclivirus. Herpes virus, Picoronovirus, or Coronavirus. The remainder of features and example features of the 3^{rd} example embodiment are as they are defined with respect to its various aspects. The remainder of features and example features of the 3^{rd} example embodiment are as they are defined with respect to its various aspects.

In a 11^{th} aspect of the 3^{rd} example embodiment, the inflammatory disease is a bacterial disease caused by gram negative bacteria, gram positive bacteria, aerobic bacteria, anaerobic bacteria, or antibiotic-resistant bacteria. The remainder of features and example features of the 3^{rd} example embodiment are as they are defined with respect to its various aspects.

In a 12^{th} aspect of the 3^{rd} example embodiment, the inflammatory disease is fungemia, fungal keratitis, candidiasis, tinea pedis, or tinea cruris. The remainder of features and example features of the 3^{rd} example embodiment are as they are defined with respect to its various aspects.

In a 13^{th} aspect of the 3^{rd} example embodiment, wherein the inflammatory disease is amoebiasis, giardiasis, toxoplasmosis, or toxocara. The remainder of features and example features of the 3^{rd} example embodiment are as they are defined with respect to its various aspects.

In a 14^{th} aspect of the 3^{rd} example embodiment, the inflammatory disease is idiopathic pulmonary fibrosis, myelofibrosis, hepatic fibrosis, cardiac fibrosis with dystolic dysfunction and CHF, kidney fibrosis, retinal fibrosis, dermal fibrosis, and scarring. The remainder of features and example features of the 3^{rd} example embodiment are as they are defined with respect to its various aspects.

In a 15^{th} aspect of the 3^{rd} example embodiment, the inflammatory disease is sepsis, cytokine storm, or sickle cell disease. The remainder of features and example features of the 3^{rd} example embodiment are as they are defined with respect to its various aspects.

In a 16^{th} aspect of the 3^{rd} example embodiment, the sialic acid ligand is an agonist of one or more of Siglec 3, 5, 7, 8, 9, 10, 11, or 15, and an agonist of one or more of Siglec 14 or 16. The remainder of features and example features of the 3^{rd} example embodiment are as they are defined with respect to its various aspects.

In a 4^{th} example embodiment, the present invention is a method of treating a subject suffering from cancer, comprising administering to the subject a therapeutically effective amount of a particle according to any aspect of the 1^{st} example embodiment.

In a 1^{st} aspect of the 4^{th} example embodiment, the cancer is a primary lung cancer, a metastatic lung cancer, a breast cancer, a colon cancer, a brain cancer, an oral cancer, an esophageal cancer, a gastric cancer, a biliary tract cancer, a hepatic cancer, rhabdomyosarcoma, a colorectal cancer, a pancreatic cancer, an ovarian cancer, a uterine cancer, a cervical cancer, a testicular cancer, a prostate cancer, a renal cell cancer, a spinal cancer, a neuroblastoma, a neuroendocrine cancer, an ocular cancer, nasopharyngeal cancer, or a dermal cancer.

In a 2^{nd} aspect of the 4^{th} example embodiment, the sialic acid ligand is an antagonist of one or more of Siglec 3, 5, 7, 8, 9, 10, 11, or 15, and an agonist of one or more of Siglec 14 or 16. The remainder of features and example features of the 4′ example embodiment are as they are defined with respect to its various aspects.

In a 3^{rd} aspect of the 4^{th} example embodiment, the method further comprises administering to the subject a therapeutically amount of a checkpoint inhibitor selected from ipilimumab, nivolimumab, pebrolizumab, atezolizumab, avelumab, durvalumab, or cemiplimab. The remainder of features and example features of the 4^{th} example embodiment are as they are defined with respect to its various aspects.

In a 4^{th} aspect of the 4^{th} example embodiment, administering the particle is concurrent or sequential with a radiation therapy. The remainder of features and example features of the 4^{th} example embodiment are as they are defined with respect to its various aspects.

In a 5^{th} aspect of the 4^{th} example embodiment, the method further comprises administering to the subject a therapeutically amount of a second therapeutic agent selected from cyclophosphamide, methothrexate, 5-fluorouracil, vinorelbine, doxorubicin, cyclophosphamide, docetaxel, bleomycin, dacarbazine, mustine, vincristine, procarbazine, prednisolone, epirubicin, cisplatin, tamoxifen, taxotere, a Her2 neu inhibitors, an anti-VEGF inhibitor, an EGFR inhibitor, an ALK inhibitor, sorafenib, or a mTOR inhibitor. The remainder of features and example features of the 4^{th} example embodiment are as they are defined with respect to its various aspects.

In a 5^{th} example embodiment, the present invention is a method of treating a subject suffering from cancer comprising administering to the subject a therapeutically effective amount of a particle according to any aspect of the 1′ example embodiment. In a 1^{st} aspect of the 5^{th} example embodiment, the cancer is acute lymphoblastic leukemia, chronic myelogenous leukemia, acute myelogenous leukemia, non-Hodgkin's or Hodgkin's lymphoma.

In a 1^{st} aspect of the 5^{th} example embodiment, the method further comprises administering to the subject a therapeutically effective amount of daunorubicin, cytarabine, or imatinib.

In a 2^{nd} aspect of the 5^{th} example embodiment, administering the particle is concurrent with or sequential with stem cell transplant or bone marrow transplant. The remainder of features and example features of the 5^{th} example embodiment are as they are defined with respect to its various aspects.

In a 6^{th} example embodiment, the present invention is a method of treating a subject suffering from an infectious disease, comprising administering to the subject a therapeutically effective amount of a particle according to any aspect of the 1 example embodiment. In a 1^{st} aspect of the 6^{th} example embodiment, the infectious disease is caused by Streptococcus group B. Streptococcus pneumonia, E. coli, Pseudomonas aeruginosa, Neisseria meningitidis, Campylobacter jejuni, Tyrpanosoma cruzi, HIV, influenza A. B, or C, Sars CoV 1, Sars Co V2, or Herpes viridae.

In a 2^{nd} aspect of the 6^{th} example embodiment, the sialic acid ligand is an agonist or antagonist of one or more of Siglec 3, 5, 7, 8, 9, 10, 11, or 15, and an agonist or antagonist of one or more of Siglec 14 or 16. The remainder of features and example features of the 6^{th} example embodiment are as they are defined with respect to its various aspects.

In a 3^{rd} aspect of the 6^{th} example embodiment, the method further comprises administering to the subject a therapeutically effective amount of one or more of zanamivir, oseltamivir, valcyclovir, acyclovir, or zidovudine. The remainder of features and example features of the 5^{th} example embodiment are as they are defined with respect to its various aspects.

In a 4^{th} aspect of the 6^{th} example embodiment, the sialic acid ligand is cognate to Siglec 11. The remainder of features and example features of the 6^{th} example embodiment are as they are defined with respect to its various aspects.

In a 5^{th} aspect of the 6^{th} example embodiment, the sialic acid ligand is cognate to Siglec 9. The remainder of features and example features of the 6^{th} example embodiment are as they are defined with respect to its various aspects.

In a 6^{th} aspect of the 6^{th} example embodiment, the sialic acid ligand is cognate to Siglec 7. The remainder of features and example features of the 6^{th} example embodiment are as they are defined with respect to its various aspects.

In a 7^{th} aspect of the 6^{th} example embodiment, the sialic acid ligand is cognate to Siglec 5. The remainder of features and example features of the 6^{th} example embodiment are as they are defined with respect to its various aspects.

In a 7^{th} example embodiment, the present invention is a method of modulating a cell-mediated inflammatory response in an immune cell, comprising contacting the immune cell with a particle according to any aspect of the 1^{st} example embodiment.

In an 8^{th} example embodiment, the present invention is a pharmaceutical composition comprising a particle according to any aspect of the 1^{st} example embodiment and a pharmaceutically acceptable carrier. In a 1^{st} aspect of the 8^{th} example embodiment, the pharmaceutically acceptable carrier includes a PBS buffer or a saline solution.

In a 2^{nd} aspect of the 8^{th} example embodiment, the concentration the particles in the carrier is from 0.01 mg/ml to 10 mg/ml. The remainder of features and example features of the 8^{th} example embodiment are as they are defined with respect to its various aspects.

In a 9^{th} example embodiment, the present invention is a composition comprising the lyophilized or freeze-dried particle according to any aspect of the 1^{st} example embodiment.

In a 10^{th} example embodiment, the present invention is a method of manufacturing a particle, comprising: reacting a biocompatible polymer scaffold P, the biocompatible polymer scaffold P comprising a first labile moiety, and a glycan G, the glycan G comprising a second labile moiety, under the condition sufficient to produce an adduct L of the first labile moiety and a second labile moiety, thereby producing a molecule represented by the following structural formula:

P-L-G

wherein P comprises at least one biocompatible polymer selected from the group consisting of polyglycolic acid, poly(L-lactic acid), poly(lactic-co-glycolic acid), polycaprolactone, poly(3-hydroxybutyric acid), poly(ethylene glycol), polyethylene oxide, Pluronic F127, Pluronic F68, poloxamer, poly(hydroxymethylmethacrylate), polyvinyl alcohol, poly(vinylpyrrolidone), hyaluronic acid, heparin, heparin sulfate, sialic-acid and chitosan; and G is a polysialic acid (PSA) comprising from 5 to 200 repeat units of sialic acid.

In a 1^{st} aspect of the 10^{th} example embodiment, the first and the second labile moieties are selected from an amine, a carboxylic acid, an azide, an alkyne, a TCO, a tetrazine, a DCBO, and a dihydrazide.

In a 1^{st} aspect of the 10^{th} example embodiment, the first and the second labile moieties are selected from an azide, an alkyne, or a tetrazine, the adduct L comprises an alkyne-azide adduct or an alkyne-azide adduct, and wherein the conditions sufficient to produce the adduct are the conditions for: a copper(I)-catalyzed azide-alkyne reaction (CuAAC); a copper-free azide-alkyne reaction: a strain-promoted azide-alkyne reaction (SPAAC); a tetrazine-alkene ligation reaction; and a TCO-tetrazine reaction. The remainder of features and example features of the 10^{th} example embodiment are as they are defined with respect to its various aspects.

In a 2^{nd} aspect of the 10^{th} example embodiment. P comprises a PLGA-PEG copolymer. The remainder of features and example features of the 10^{th} example embodiment are as they are defined with respect to its various aspects.

In an 11^{th} example embodiment, the present invention is a method of inhibiting complement activation in a subject, the method comprising administering to the subject a therapeutically effective amount of a particle according to any of the aspect of the 1^{st} example embodiment.

In a 1^{st} aspect of the 11^{th} example embodiment, the subject produces excessive Complement Component C3, C3b.

In a 2^{nd} aspect of the 11^{th} example embodiment, the particle is an agonist of Siglec 11. The remainder of features and example features of the 11^{th} example embodiment are as they are defined with respect to its various aspects.

In a 3^{rd} aspect of the 11^{th} example embodiment, the particles binds Complement Factor H. The remainder of features and example features of the 11^{th} example embodiment are as they are defined with respect to its various aspects.

In a 12^{th} example embodiment, the present invention is a method of treating a subject suffering from complement hyperactivation disease, comprising administering to the subject a therapeutically effective amount of a particle according to any aspect of the 1^{st} example embodiment.

In a 1^{st} aspect of the 12^{th} example embodiment, the route of administration is one or more of intravenous, intravitreal, oral, oral rinse, intraocular, subretinal, subtenons, intrascleral, periocular, inhalational nasal and oral, intramuscular, intra-areterial, intraspinal, intrathecal, intracranial, intradermal, transdermal (topical), transmucosal, subcutaneous, pulmonary lavage, gastric lavage, and intrahepatic, subcutaneous, rectal, intra-articular.

In a 2^{nd} aspect of the 12^{th} example embodiment, the complement hyperactivation disease is dry and wet macular degeneration, paroxysmal nocturnal hematuria, systemic lupus erythematosis, sepsis, anti-phospholipid syndrome, alzheimers, stroke, myocaridal infarction, shock, organ transplant rejection, biomaterial implant rejection, COPD exacerbation, gingivitis/periodontal disease, systemic inflammatory response syndrome. The remainder of features and example features of the 11^{th} example embodiment are as they are defined with respect to its various aspects.

### Synthetic Schemes

### Synthetic Example 1

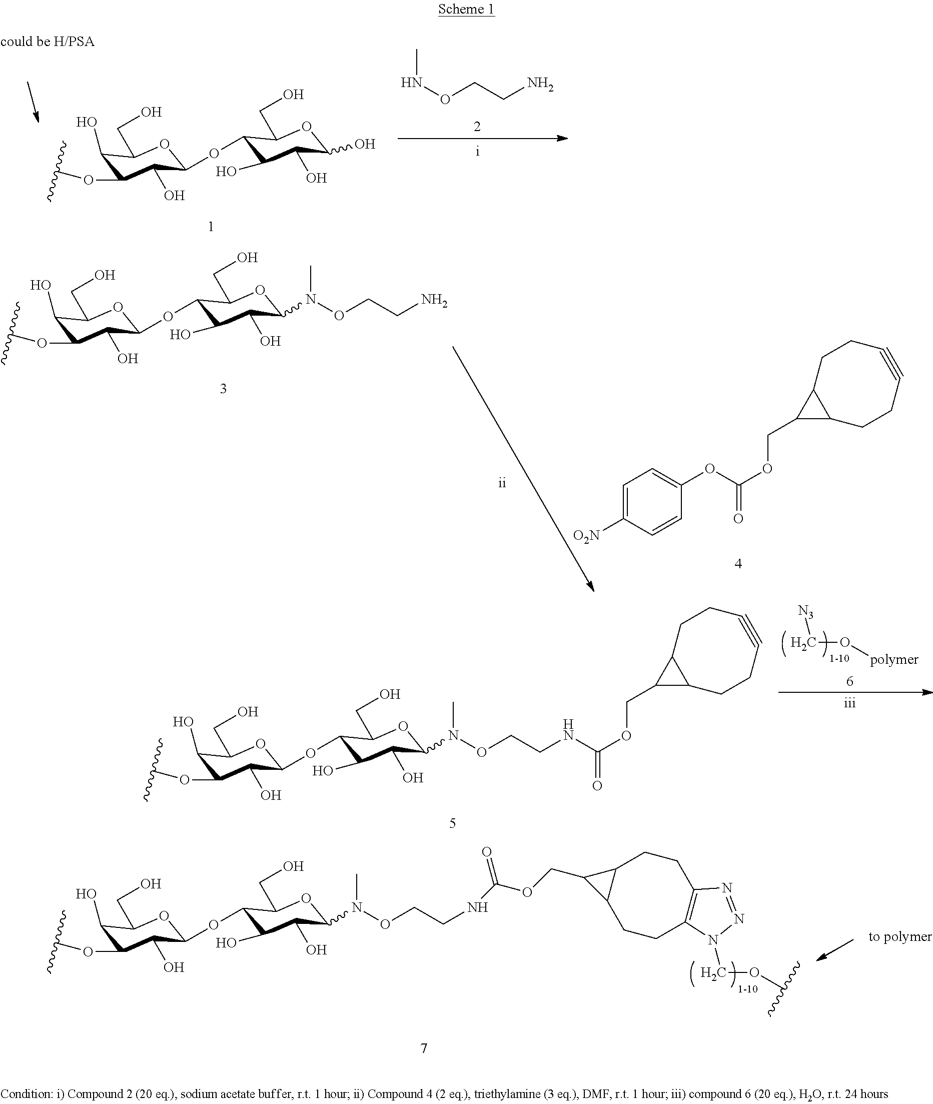

Compound 3: To a flask, compound 1 (1 eq.), linker 2 (20 eq.) and sodium acetate buffer were added and stirred at room temperature for 1 hour. The mixture was purified by P2 bio-gel filtration using 0.1 M ammonium bicarbonate as eluent to yield compound 3.

Compound 5: Compound 3 (1 eq.), BCN—PNP 4 (2 eq.), triethylamine (3 eq.) and DMF were added into a flask and the mixture was stirred at room temperature for 1 hour. Purification of the product by C18 column chromatography get compound 5.

Compound 7: Compound 5 (1 eq.), H₂O, azido polymer 6 (20 eq.) were added into a flask and the mixture was stirred at room temperature for 24 hours. The product was purified by size-exclusion chromatography to get compound 7.

### Synthetic Example 2

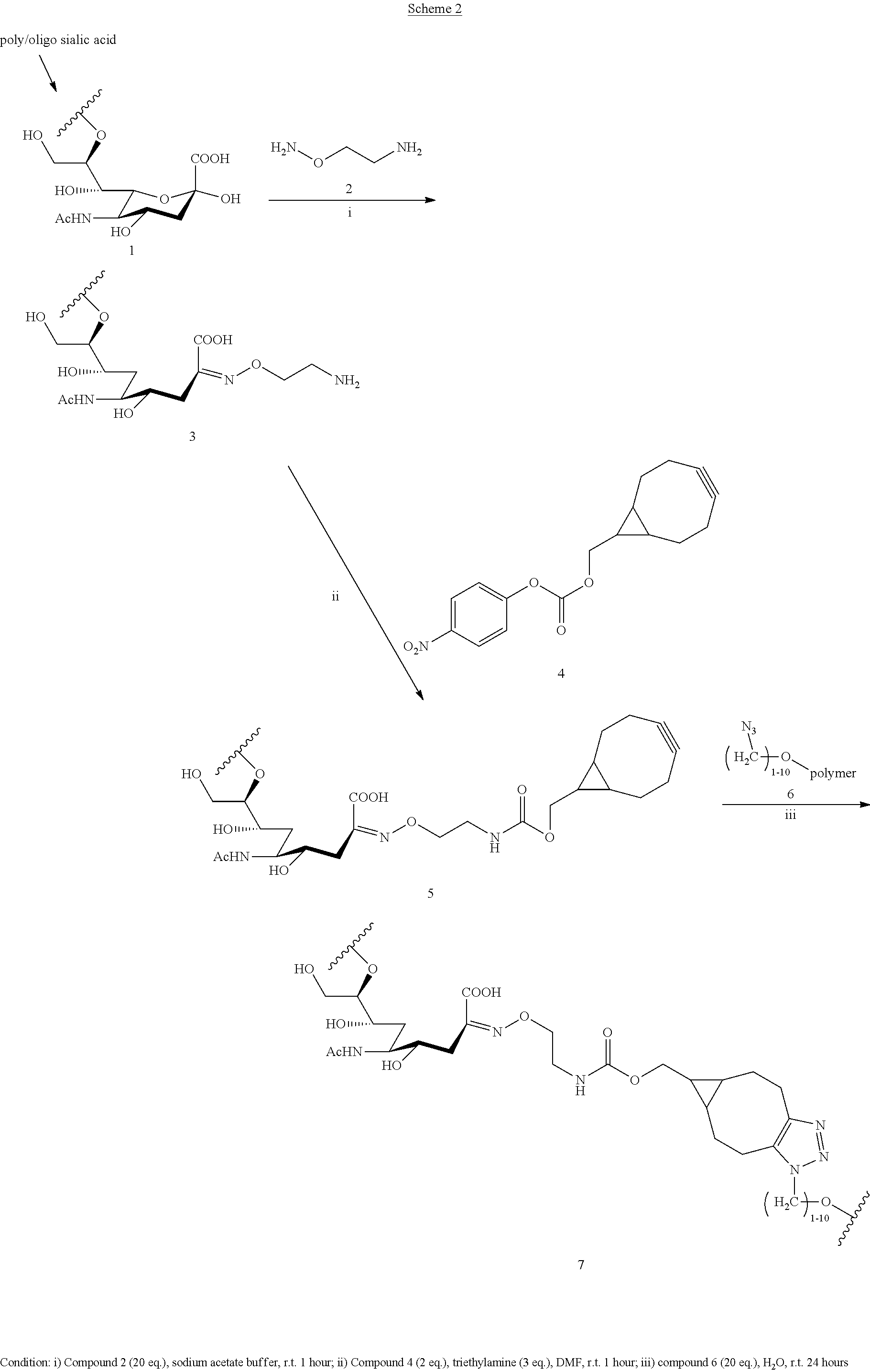

Compound 3: To a flask, compound 1 (1 eq.), linker 2 (20 eq.) and sodium acetate buffer were added and stirred at room temperature for 1 hour. The mixture was purified by P2 bio-gel filtration using 0.1M ammonium bicarbonate as eluent to yield compound 3.

Compound 5: Compound 3 (1 eq.), BCN—PNP 4 (2 eq.), triethylamine (3 eq.) and DMF were added into a flask and the mixture was stirred at room temperature for 1 hour. Purification of the product by C18 column chromatography get compound 5.

Compound 7: Compound 5 (1 eq.), H₂O, azido polymer 6 (20 eq.) were added into a flask and the mixture was stirred at room temperature for 24 hours. The product was purified by size-exclusion chromatography to get compound 7.

### Synthetic Example 3

Lactose modified with various appropriate linker such as aminoalkyl or amino oligo-ethyleneglycol can be prepared by chemical methods (Nat. Chem. 1, 611-622, 2009). The resulting compounds can be extended by a polysialic acid (PSA) employing microbial or mammalian sialyl transferases (Glycobiology, 18, 177-186, 2008). Examples of such modified lactoses are those represented by the following structural formulas:

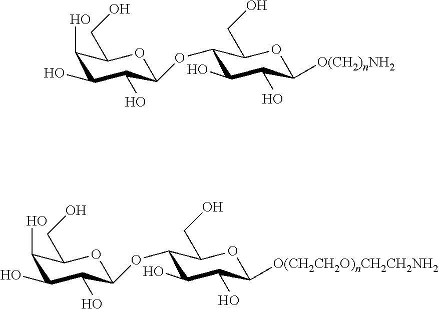

The amine of the linker provides a functional group for attached to polymers having an activated ester such as a N-hydroxysuccinimide (NHS) (Chem. Rev. 2016, 116, 3, 1434-1495). The amine can be converted into various functional groups for attachment to appropriately functionalized polymers. For example, the amine can be converted into an azide by an azido transfer reaction (ACS Comb. Sci. 2013, 15, 7, 331-334), which can then be conjugated with a polymer modified by a cyclooctyne such as BCN or DBCO (Accounts Chemical Research, 9, 805-815, 2011). Alternatively, the amine can be employed to install a BCN or DBCO moiety by employing activated ester or carbamates of these reagents. The resulting can be linked to polymers modified by an azido moiety. A thiol can be installed by reaction of the amine with S-(2-bromo-2-oxoethyl) ethanethioate followed by treatment with mild base. The thiol can then be reacted with maleimide modified polymers using standard conditions.

Microbes have been described that that can biosynthesize PSA linked to a lactose moiety (Richard et al. Glycobiology, 2016, vol. 26, no. 7, 723-731). In this case, an appropriate linker needs to be attached for conjugation to an appropriately functionalized polymer. This can be accomplished by reductive amination or treatment with an appropriately functionalized hydroxylamine (Glycobiology. 2006:16:21C-27C, and Chem. Commun., 2014, 50, 7132-7135). In this way an amino group can be installed that can be employed for direct conjugation to active ester functionalized polymer or derivatized as described above. An example synthetic scheme is shown below:

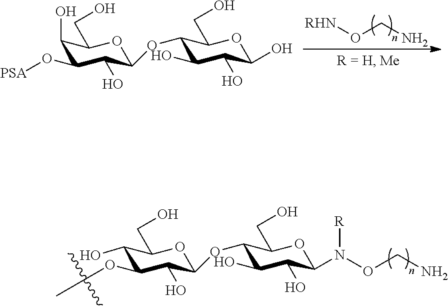

An amino group can also be installed at the non-reducing end as described in US patent 2015/0150997, the entire teachings of which are incorporated herein by reference.

Treatment of PSA with sodium periodate as described in US patent 2015/0150997 installs an aldehyde at the non-reducing end that can be employed to install various functional groups (i.e. amine, see Scheme 1) via reductive amination (Bioconjug Chem. 2008; 19(7): 1485-1490) or oxime or hydrazone ligation (Chem. Rev. 2017, 117, 15, 10358-10376, and Nat. Methods. 2009, 6(3): 207-209). See the following synesthetic Scheme 3 for non-reducing end modification of PSA:

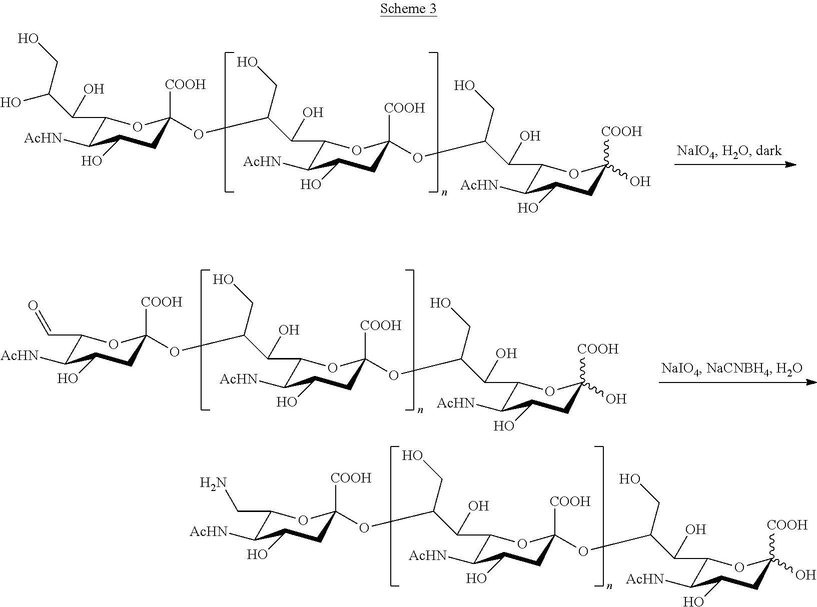

PSA can also be modified at the anomeric center with amino containing linker by reaction with phenylenediamine derivative (Glycobiology, 2016, vol. 26, no. 7, 723-731) to give a fluorescent quinoxalinone with amino terminus which could be further extended via chemistry mentioned above. See Scheme 4 below: PSA compound 1 could react with phenylenediamine 2 derivative functionalized with amino terminus, forming a fluorescent 3 which could be modified by various functional groups such as NHS functionalized polymer.

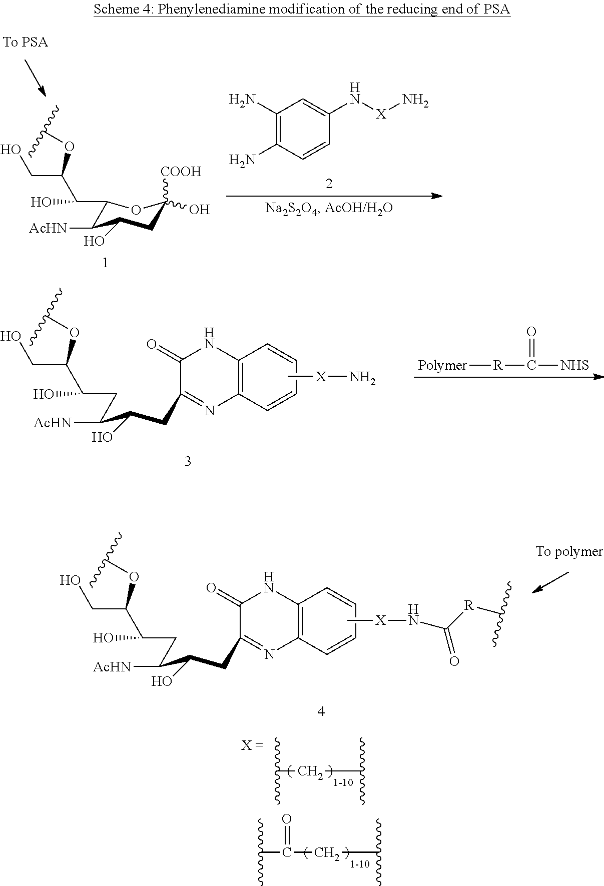

### Synthetic Example 4

The following reactions can be used to conjugate the PSA and the polymer via a triazole link.

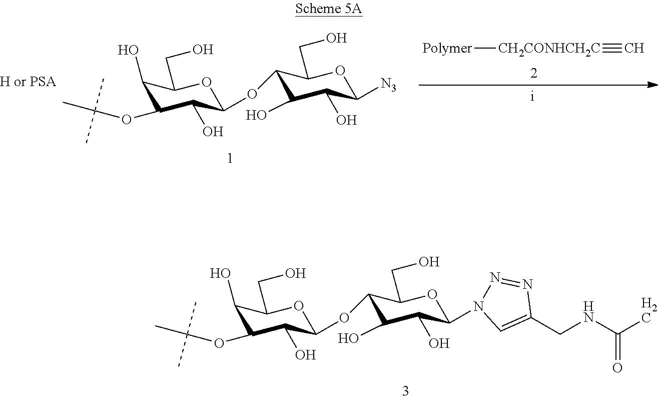

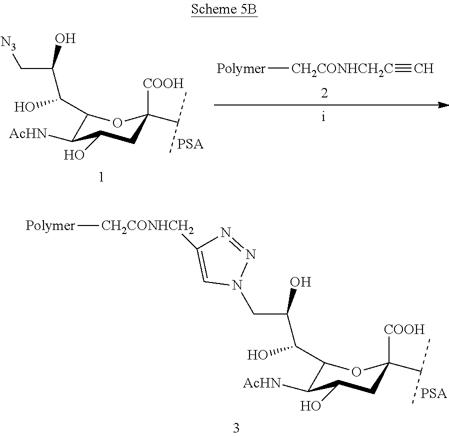

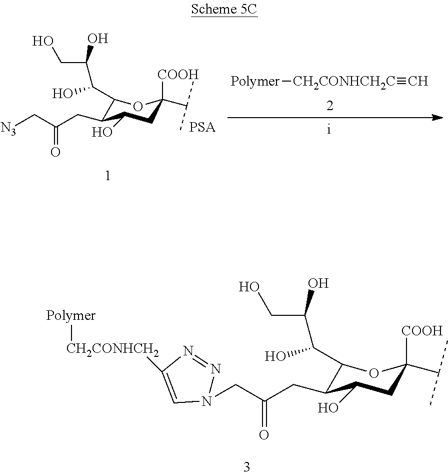

Conditions for each of the reactions shown in Schemes 5A through 5C are: CuSO4, THPTA, ascorbic acid

### Synthetic Example 5

The following reactions can be used to conjugate the PSA and the polymer via a DBCO link.

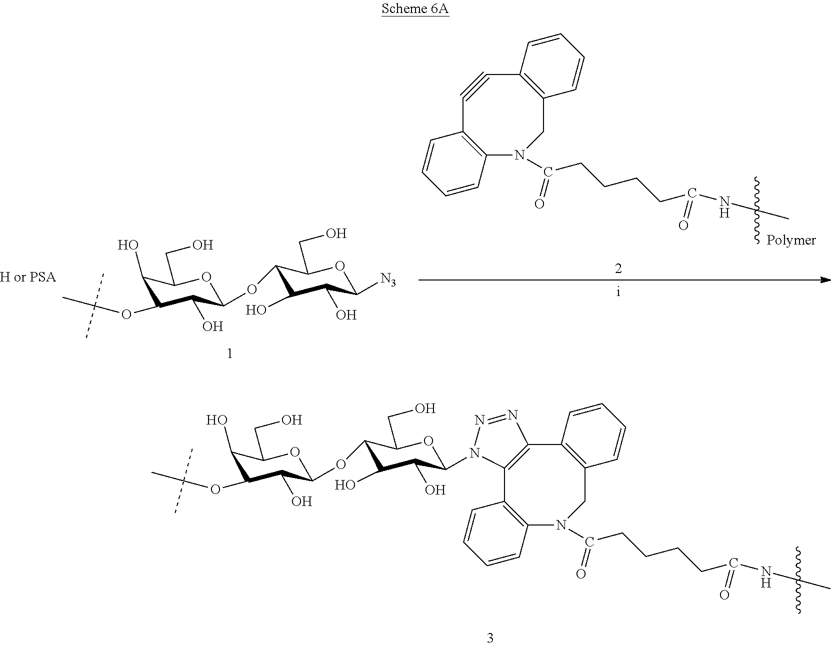

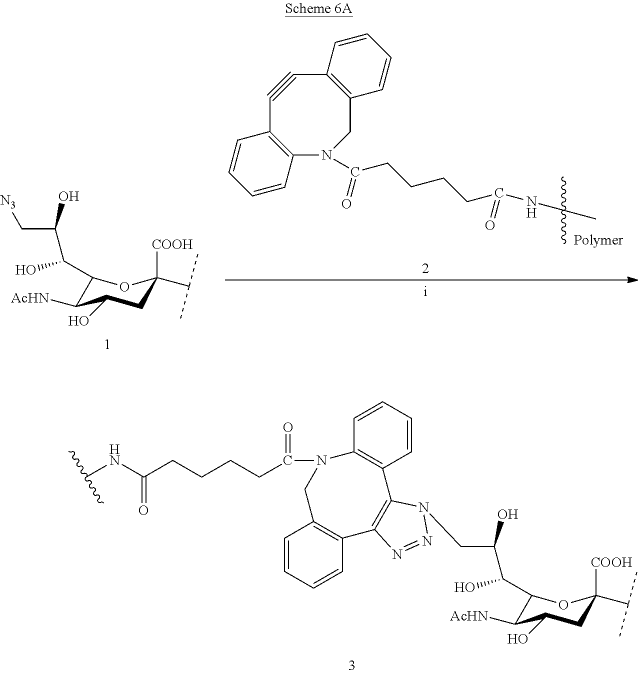

Conditions for each of the reactions shown in Schemes 6A and 6B are: H₂O, r.t. 24 hours.

### Synthetic Example 6

The following reactions can be used to conjugate the PSA and the polymer via a thio-maleimide link.

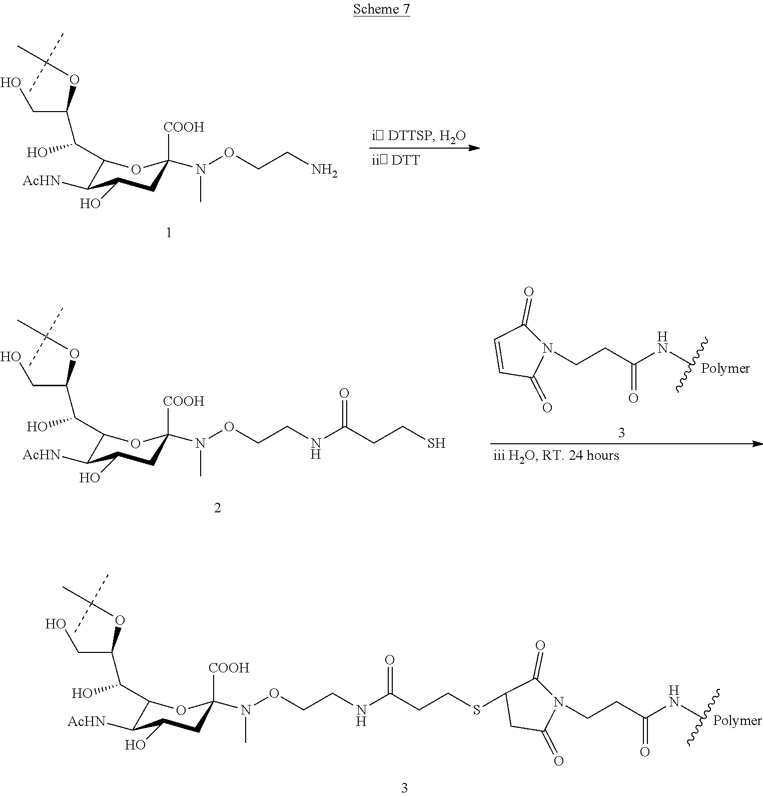

### Additional Embodiments

In certain embodiments, the present invention is defined by the following numbered example embodiments.

1. A particle, comprising:
a biocompatible polymer scaffold; and a glycan covalently attached to the scaffold, wherein the glycan comprises at least one sialic acid ligand, wherein the at least one sialic acid ligand comprises five or more carbohydrate residues.

2. The particle of Claim 1, wherein the average molecular weight of the glycan is from 900 Da to 100,000 Da.

3. The particle of Claim 1, wherein the particle is a nanoparticle.

4. The particle of any one of Claims 1-3, comprising at least a first sialic acid ligand and at least a second sialic acid ligand different from the first sialic acid ligand.

5. The particle of any one of Claims 1-4, wherein the at least one sialic acid ligand is cognate to a Siglec receptor selected from Sigelc 1, Siglec 2, Siglec 3, Siglec 4, Siglec 5, Siglec 6, Siglec 7, Siglec 8, Siglec 9, Siglec 10, Siglec 11, Siglec 12, Siglec 13, Siglec 14, Siglec 15, Siglec 16, Siglec 17, Siglec E. Siglec F, or Siglec H.

6. The particle of any one of Claims 1-4, wherein the at least one sialic acid ligand is cognate to a receptor selected from platelet immunoglobulin-like type 2 receptor (PILR-alpha or PILR-beta), platelet endothelial cell adhesion molecule (PECAM-1), neural cell adhesion molecule (NCAM) and basigin (CD147).

7. The particle of any one of Claims 1-4, wherein the at least one sialic acid ligand is cognate to a receptor derived from an infectious agent.

8. The particle of any one of Claims 1-4, wherein the biocompatible polymer of the scaffold comprises a biodegradable polymer.

9. The particle of Claim 8, wherein the biodegradable polymer is selected from the group consisting of polyglycolic acid, poly(L-lactic acid), poly(lactic-co-glycolic acid), polycaprolactone, and poly(3-hydroxybutyric acid).

10. The particle of any one of Claims 1-4, wherein the scaffold comprises a non-biodegradable polymer.

11. The particle of any one of Claims 1-4, wherein the scaffold comprises a polymer selected from the group consisting of poly(ethylene glycol), polyethylene oxide, Pluronic F127, Pluronic F68, poloxamer, poly(hydroxymethylmethacrylate), polyvinyl alcohol, poly(vinylpyrrolidone), hyaluronic acid, heparin, heparin sulfate, sialic-acid and chitosan.

12. The particle of any one of Claims 1-4, wherein the scaffold comprises a block copolymer PLGA-PEG.

13. The particle of Claim 12, wherein an average molecular weight of the PEG block is from 900 to 50,000 Da.

14. The particle of Claim 13, wherein the average molecular weight of the PEG is from 1,000 Da to 5000 Da.

15. The particle of any one of Claims 1-14, wherein the particle is non-spherical and has an average cross-sectional width of at least 50 nm and not greater than 750 nm.

16. The particle of any one of Claims 1-14, wherein the particle is spherical, and has a diameter of 20-2000 nm.

17. The particle of any one of Claims 1-14, wherein the particle is non-spherical, and wherein an average greatest cross-sectional width of the particle in from 1 micron to 5 microns

18. The particle of any one of Claims 1-17, wherein the sialic-acid ligand comprises one or more of Neu5Ac and Neu5Gc.

19. The particle of any one of Claims 1-18, wherein the sialic-acid ligand is a sialic acid polymer of one or both of Neu5Ac and Neu5Gc.

20. The particle of any one of Claims 11-17, wherein the sialic-acid ligand is covalently attached to the biocompatible polymer by a glycosidic a2-3, a2-6, a2-8, or a2-9 bond.

21. The particle of any one of Claims 1-4, wherein the sialic acid ligand is a polysaccharide having a degree of polymerization from DP5 to DP200.

22. The particle of any one of Claims 1-4, wherein the sialic acid ligand is a linear or branched polysaccharide.

23. The particle of any one of Claims 1-4, wherein the sialic-acid ligand is a polysaccharide of two or more of GlcNAc, GluNAc. GalNAc, ManAc, Fucose, and wherein the polysaccharide is terminated with a sialic acid.

24. The particle of any one of Claims 1-4, further comprising at least one second therapeutic agent.

25. The particle of Claim 27, wherein the second therapeutic agent selected from an antibody, a polypeptide, a lipid, a small molecule, or a sialic-acid modified cell.

26. The particle of any one of Claims 1-4, wherein the sialic-acid ligand is present at a density of 0.0001-10 molecules per nm² of the particle.

27. The particle of any one of Claims 1-4, wherein the sialic-acid ligand is cognate to Siglecs 3-16.

28. The particle of Claim 27, wherein Siglec 3-16 are found on the immune cells selected from the group consisting of macrophages, microglia, dendritic cells, NK cells, neutrophils, polymorphonuclear cells, T-cells CD8, T-Cells CD34, basophils, mast cells, and eosinophils.

29. The particle of Claim 28, wherein the immune cell is an activated macrophage and the at least one sialic acid ligand is cognate to Siglec 3, 5, 7, 9, 11, 12, 15 or 16.

30. The particle of Claim 28, wherein the immune cell is an activated monocyte and the at least one sialic acid ligand is cognate to Siglec 3, 5, 7, 9, 10, 13 or 14.

31. The particle of Claim 28, wherein the immune cell is an activated dendritic cell, and the at least one sialic acid ligand is cognate to Siglec 3, 7, or 9.

32. The particle of Claim 28, wherein the immune cell is an activated neutrophil, and the at least one sialic acid ligand is cognate to Siglec 3, 5, 9 or 14.

33. The particle of Claim 28, wherein the immune cell is an activated natural killer cell (NK), and the at least one sialic-acid ligand is cognate to Siglec 7.

34. The particle of Claim 28, wherein the immune cell is an activated B cell, and the at least one sialic-acid ligand is cognate to Siglecs 5 or 10.

35. The particle of Claim 28, wherein the immune cell is an activated CD8+ T-cell, and the at least one sialic-acid ligand is cognate to Siglecs 7 or 9.

36. The particle of Claim 28, wherein the immune cell is an activated CD34+ T-cell, and the at least one sialic-acid ligand is cognate to Siglecs 3, 5, 9, or 10.

37. The particle of Claim 28, wherein the immune cell is an eosinophil, and the at least one sialic-acid ligand is cognate to Siglecs 7, 8, or 10.

38. The particle of Claim 28, wherein the immune cell is an activated mast cell, and the at least one sialic-acid ligand is cognate to Siglecs 3, 5, 6, or 8.

39. The particle of Claim 28, wherein the immune cell is an activated basophil, and the at least one sialic-acid ligand is cognate to Siglecs 3, 5, 6, 8, or 14.

40. The particle of Claim 28, wherein the immune cell is an microglial cell, and the at least one sialic-acid ligand is cognate to Siglec 11.

41. The particle of any one of Claims 1-4, wherein the at least one sialic-acid ligand is cognate to complement factor H at the complement control protein (CCP) regions 4-6 and 19-20.

42. The particle of any one of Claims 1-4, wherein the at least one sialic acid ligand includes an inhibitor of a neuraminidase or a sialidase.

43. The particle of Claim 42, wherein the neuraminidase or the sialidase is an influenza A, B or C viral neuraminidase or sialidase.

44. The particle of Claim 42, wherein the neuraminidase or the sialidase is a neuraminidase or a sialidase expressed in a respiratory tract epithelial cell.

45. The particle of any one of Claims 1-4, wherein the at least one sialic-acid ligand is cognate to a viral capsid sialic acid binding site.

46. The particle of Claim 45, wherein the viral capsid sialic acid binding site is a hemagglutinin esterase on an Influenza A, B, or C virus.

47. The particle of Claim 45, wherein the viral capsid sialic acid binding site is a host attachment/infectivity site of a Spike protein of SARS-CoV1 or SARS-CoV2 virus.

48. The particle of any one of Claims 1-4, wherein: the biocompatible polymer is a PLGA-PEG copolymer; the sialic acid ligand is represented by the following structural formula:

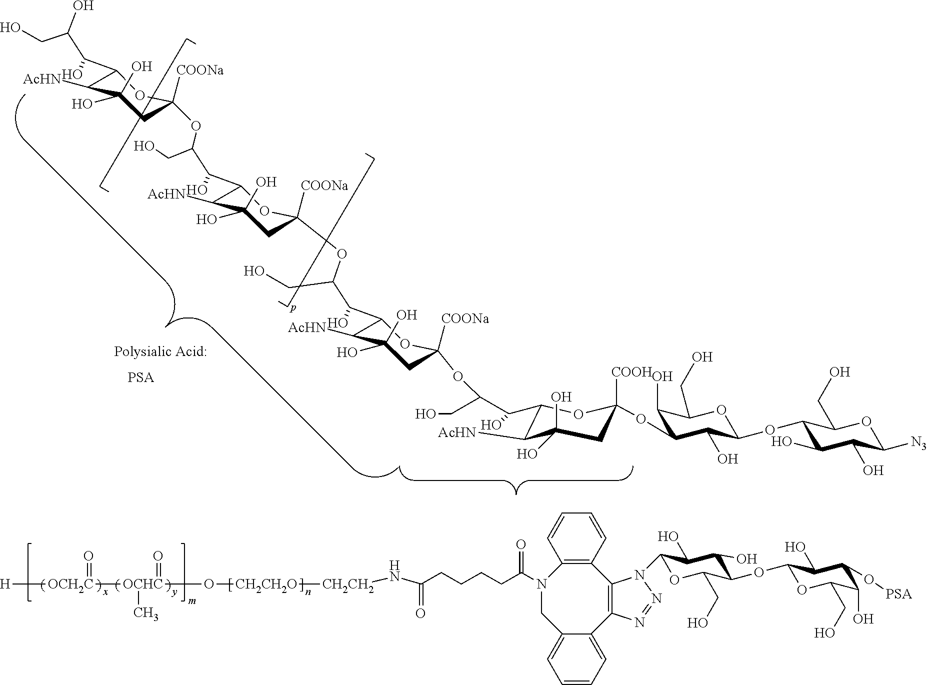

wherein x is an integer from 0 to 10, y is an integer from 0 to 10, m is an integer from 1 to 500, n is an integer from 5 to 450, and p is an integer from 1 to 200, provided that x and y are not simultaneously 0.

49. A method of modulating a cell-mediated inflammatory response in an immune cell, comprising: contacting the immune cell with a particle of any one of Claims 1-48.

50. A method of treating a subject suffering from an inflammatory disease, comprising: administering to the subject a therapeutically effective amount of a particle of any one of Claims 1-48.

51. The method of Claim 50, wherein the route of administration is one or more of intravenous, intravitreal, oral, intraocular, subretinal, subtenons, intrascleral, periocular, inhalational nasal and oral, intramuscular, intra-areterial, intraspinal, intrathecal, intracranial, intradermal, transdermal (topical), transmucosal, subcutaneous, pulmonary lavage, gastric lavage, and intrahepatic, subcutaneous, or rectal.

52. The method of Claim 50, wherein the inflammatory disease is tuberculosis, chronic obstructive pulmonary disorder (COPD), asthma, acute lung injury, acute respiratory distress syndrome, cystic fibrosis, bronchiectasis, pulmonary fibrosis interstitial lung disease, pulmonary vascular disease, influenza, viral pneumonia, bacterial pneumonia, allergic bronchitis, nonallergic bronchitis, rhinitis, or fibrosing alveolitis.

53. The method of Claim 50, wherein the inflammatory disease is rheumatoid arthritis, fibromyalgia, systemic lupus erythematosus, systemic sclerosis (scleroderma), psoriatic arthritis, ankylosing spondylitis, sjogrens syndrome, polymyalgia rehumatica, gout, osteoarthritis, infectious arthritis or juvenile idiopathic arthritis.

54. The method of Claim 50, wherein the inflammatory disease is Crohn's disease, ulcerative colitis, irritable bowel syndrome, celiac disease, diverticulitis, gastroesophageal reflux, lactose intolerance, peptic ulcer, cholecystitis, gastritis, colitis, pancreatitis, autoimmune hepatitis, hepatitis, infectious hepatitis, or pancreatitis.

55. The method of Claim 50, wherein the inflammatory disease is septic shock, atherosclerosis, diastolic dysfunction, heart failure, cardiac fibrosis, coxsackie myocarditis, congenital heart block, autoimmune myocarditis, or giant cell myocarditis.

56. The method of Claim 50, wherein the inflammatory disease is kidney transplant rejection, glomerulonephritis, acute nephritis, cystitis, prostatitis, diabetic nephritis, or diabetic kidney disease.

57. The method of Claim 50, wherein the inflammatory disease is dermatitis, eczema, inflammatory rashes, scleroderma, keloid, acne, sarcoidosis, tinea cruris, tinea corporis, tinea pedis, tinea capitis, tinea unguium, rosacea, vitiligo, lichen sclerosis, autoimmune urticaria, dermatomyositis, or hidradenitis suppurativa.

58. The method of Claim 50, wherein the inflammatory disease is diabetes, SLE, multiple sclerosis, sjogrens syndrome, Addison's disease, Graves Disease, Hashimotos thyroiditis, myasthenia gravis, autoimmune vasculitis, celiac disease, pomaceous anemia, alopecia areata, autoimmune hepatitis, autoimmune angioedema, autoimmune encephalomyelitis, autoimmune inner ear disease, Guillain barre, Kawasaki disease, lambert-eaton syndrome, Vogt-Koyanagi-Harada Syndrome, systemic vasculitis, giant cell arteritis, sarcoidosis, or polyarteritis nodosa.

59. The method of Claim 50, wherein the inflammatory disease is neuromyelitis, multiple sclerosis, encephalitis, neuro sarcoid, Alzheimers, amyotrophic lateral sclerosis, or Huntington's chorea, transverse myelitis, Guillain barre syndrome, Parkinsons disease, or benign essential tremors.

60. The method of Claim 50, wherein the inflammatory disease is a viral disease caused by influenza A, influenza B, influenza C, SARS-CoV1, SARS-CoV2, Newcastle Disease virus, Sendai virus, Polyomavirus, HIV, Flavivirus, Caclivirus, Herpes virus, Picoronovirus, or Coronavirus.

61. The method of Claim 50, wherein the inflammatory disease is a bacterial disease caused by gram negative bacteria, gram positive bacteria, aerobic bacteria, anaerobic bacteria, or antibiotic-resistant bacteria.

62. The method of Claim 50, wherein the inflammatory disease is fungemia, fungal keratitis, candidiasis, tinea pedis, or tinea cruris.

63. The method of Claim 50, wherein the inflammatory disease is amoebiasis, giardiasis, toxoplasmosis, or toxocara.

64. The method of Claim 50, wherein the inflammatory disease is idiopathic pulmonary fibrosis, myelofibrosis, hepatic fibrosis, cardiac fibrosis with dystolic dysfunction and CHF, kidney fibrosis, retinal fibrosis, dermal fibrosis, and scarring.

65. The method of Claim 50, wherein the inflammatory disease is sepsis, cytokine storm, or sickle cell disease.

66. The method of Claim 50, wherein the sialic acid ligand is an agonist of one or more of Siglec 3, 5, 7, 8, 9, 10, 11, or 15, and an agonist of one or more of Siglec 14 or 16.

67. A method of treating a subject suffering from cancer, comprising: administering to the subject a therapeutically effective amount of a particle of any one of Claims 1-48, wherein the cancer is a primary lung cancer, a metastatic lung cancer, a breast cancer, a colon cancer, a brain cancer, an oral cancer, an esophageal cancer, a gastric cancer, a biliary tract cancer, a hepatic cancer, rhabdomyosarcoma, a colorectal cancer, a pancreatic cancer, an ovarian cancer, a uterine cancer, a cervical cancer, a testicular cancer, a prostate cancer, a renal cell cancer, a spinal cancer, a neuroblastoma, a neuroendocrine cancer, an ocular cancer, nasopharangeal cancer, or a dermal cancer.

68. The method of Claim 67, wherein the sialic acid ligand is an antagonist of one or more of Siglec 3, 5, 7, 8, 9, 10, 11, or 15, and an agonist of one or more of Siglec 14 or 16.

69. The method of Claim 67, further comprising administering to the subject a therapeutically amount of a checkpoint inhibitor selected from ipilimumab, nivolimumab, pebrolizumab, atezolizumab, avelumab, durvalumab, or cemiplimab.

70. The method of Claim 67, wherein administering the particle is concurrent or sequential with a radiation therapy.

71. The method of Claim 67, further comprising administering to the subject a therapeutically amount of a second therapeutic agent selected from cyclophosphamide, methothrexate, 5-fluorouracil, vinorelbine, doxorubicin, cyclophosphamide, docetaxel, bleomycin, dacarbazine, mustine, vincristine, procarbazine, prednisolone, epirubicin, cisplatin, tamoxifen, taxotere, a Her2 neu inhibitors, an anti-VEGF inhibitor, an EGFR inhibitor, an ALK inhibitor, sorafenib, or a mTOR inhibitor.

72. A method of treating a subject suffering from cancer, comprising: administering to the subject a therapeutically effective amount of a particle of any one of Claims 1-48, wherein the cancer is acute lymphoblastic leukemia, chronic myelogenous leukemia, acute myelogenous leukemia, non-Hodgkin's or Hodgkin's lymphoma.

73. The method of Claim 72, further comprising administering to the subject a therapeutically effective amount of daunorubicin, cytarabine, or imatinib.

74. The method of Claim 72, wherein administering the particle is concurrent with or sequential with stem cell transplant or bone marrow transplant.

75. A method of treating a subject suffering from an infectious disease, comprising: administering to the subject a therapeutically effective amount of a particle of any one of Claims 1-48, wherein the infectious disease is caused by Streptococcus group B. Streptococcus pneumonia, E. coli, Pseudomonas aeruginosa, Neisseria meningitidis, Campylobacter jejuni, Tyrpanosoma cruzi, HIV, influenza A, B, or C, Sars CoV1, Sars Co V2, or Herpes viridae.

76. The method of Claim 75, wherein the sialic acid ligand is an agonist or antagonist of one or more of Siglec 3, 5, 7, 8, 9, 10, 11, or 15, and an agonist or antagonist of one or more of Siglec 14 or 16.

77. The method of Claim 76, further comprising administering to the subject a therapeutically effective amount of one or more of zanamivir, oseltamivir, valcyclovir, acyclovir, or zidovudine.

78. The method of Claim 77, wherein the sialic acid ligand is cognate to Siglec 11.

79. The method of Claim 77, wherein the sialic acid ligand is cognate to Siglec 9.

80. The method of Claim 77, wherein the sialic acid ligand is cognate to Siglec 7.

81. The method of Claim 77, wherein the sialic acid ligand is cognate to Siglec 5.

82. A method of treating a subject suffering from an ophthalmic disease, comprising: administering to the subject a therapeutically effective amount of a particle of any one of Claims 1-48.

83. The method of Claim 82, wherein the ophthalmic disease is a dry age-related macular degeneration, a wet age-related macular degeneration, non-proliferative diabetic retinopathy, proliferative diabetic retinopathy, macular edema, uveitis, dry eyes, conjunctivitis, thyroid ophthalmopathy, endophthalmitis, retinal degeneration, glaucoma, retinal vein occlusions, blepharitis, keratitis, an ocular infections, or a cataract.

84. The method of Claim 82, wherein the sialic acid ligand is an agonist of one or more of Siglec 3, 5, 7, 8, 9, 10, 11, or 15, and an antagonist of one or more of Siglec 14 or 16.

85. A pharmaceutical composition comprising a particle of any one of Claims 1-48 and a pharmaceutically acceptable carrier.

86. The pharmaceutical composition of Claim 85, wherein the pharmaceutically acceptable carrier includes a PBS buffer or a saline solution.

87. A pharmaceutical composition of Claim 85, wherein the concentration the particles in the carrier is from 0.01 mg/ml to 100 mg/ml.

88. A composition comprising the lyophilized or freeze-dried particle of any one of Claims 1-48.

89. A method of manufacturing a particle, the method comprising:
reacting a biocompatible polymer scaffold, the biocompatible polymer comprising a first labile moiety, and
a glycan, comprising at least one sialic acid ligand, wherein the glycan comprises a second labile moiety,
under the condition sufficient to produce an adduct of the first labile moiety and a second labile moiety,
wherein the sialic acid ligand comprises at least three sialic acid derivatives.

90. The method of Claim 89, wherein the first and the second labile moieties are selected from an amine, a carboxylic acid, an azide, an alkyne, a TCO, a tetrazine, a DCBO, and a dihydrazide.

91. The method of Claim 89, wherein the first and the second labile moieties are selected from an azide, an alkyne, or a tetrazine, the adduct selected from an alkyne-azide adduct or an alkyne-azide adduct and wherein the conditions sufficient to produce the adduct are the conditions for:
a copper(I)-catalyzed azide-alkyne reaction (CuAAC);
a copper-free azide-alkyne reaction;
a strain-promoted azide-alkyne reaction (SPAAC);
a tetrazine-alkene ligation reaction; and
a TCO-tetrazine reaction.

92. The method of any one of Claims 89-91, wherein the scaffold comprises a PEG polymer.

All documents, papers and published materials referenced herein, including books, journal articles, manuals, published patent applications and patents, are expressly incorporated herein by reference in their entireties.

### EXEMPLIFICATION

Table of Reagents:

| Materials | Manufacturer | Catalog | Lot Number |
|---|---|---|---|
| Benzyl Alcohol | Sigma | 108006-100mL | 01296BK |
| Cell & Molecular UltraPure Water | Intermountain Life | WPW-CMG-1X6 | 22101201 |
| Cell Culture Grade Water | Corning | 25-055-LB | 09220006 |
| CMP-Azido-Sialic acid (1 mM stock) | R&D Systems | ES102-100 | DESZ0520031 |
| CMP-Azido-Sialic acid (1.49 mg/ml) | R&D Systems | ES102-MTO | DESZ052005A |
| Colominic Acid Sodium Salt | Carbosynth | YC11298 | YC112981902; |
| | | | YC112981903; |
| | | | YC112982001 |
| Colominic Acid Sodium Salt | Sigma | C5762-1G | SLBX1715 |
| Cy3-Azide | Sigma | 777315-5mg | MKCG3741 |
| Cytidine 5′-Monophospho-N- | Sigma | C8271-5mg | SLCB1103 |
| Acetylneuraminic Acid Sodium | | | |
| Ethyl Acetate | EMD Millipore | EX0240-5 | 57325 |
| HyClone ™ Phosphate Buffered Saline | Cytiva | SH3025602 | AF29498797 |
| (PBS) | | | |
| Magnesium Chloride | Sigma | 221279 | MKCC1186 |
| (MnCl₂)Tetrahydrate | | | |
| PDLLA-FITC | NANOSOFT Polymers | 10913-10K | 109130105 |
| PLGA (Resomer RG 752 H) | Evonik | 99024540 | D170800539 |
| PLGA10k-PEG2k | Millipore Sigma | 911399-1G | MKCL4635 |
| PLGA-PEG-COOH | NANOSOFT Polymers | 2786-/10k-5k | 27861005C1; |
| | | | 27861005C2 |
| PLGA-PEG-DBCO | NANOSOFT Polymers | 9765-10k-5k | 97651005C1; |
| | | | 97651005C2 |
| Recombinant Human NCAM-1/CD56 | R&D Systems | 2408-NC-050 | NET0319041 |
| Protein, | | | |
| Recombinant Human ST8Sia4 Protein | R&D Systems | 7027-GT-020 | DAVQ0120031 |
| (stock at 0.469 mg/ml)} | | | |
| Sialic Acid (NANA) Assay Kit | AbCam | ab83375 | |
| Sialytransferase activity kit | R&D Systems | EA002 | P200221 |
| Sodium Chloride (NaCl) | Sigma | S9625-1KG | SLCC4065 |
| Sucrose | Pfanstiehl | S-124-2-MC | 40482A |
| Sodium Periodate | Sigma | 311448-5G | MKCK2840 |
| Tween 80 | JT Baker | 4117-04 | 170176 |
| 10x Dulbecco's Phosphate Buffered | Boston Bioproducts | BS5-220DM-C | D28M118 |
| Saline | | | |
| (DPBS) | | | |
| 2-(N-Morpholino)Ethanesulfonic Acid | Sigma | M8250 | SLCB9770 |
| (MES | | | |
| Hydrate) | | | |
| 4-Amino-3-Hydrazino-5-Mercapto- | Alfa Aesar | L00982-06 | |
| 1,2,4- | | | |
| Triazole | | | |

| Reagent | Catalog No | Source |
|---|---|---|
| Phorbol-12-myristate-13- | P8139 | Sigma Aldrich, St. Louis, MO |
| acetate (PMA) | | |
| Human monocytes cell | (ATCC ® TIB-202 ™) | ATCC, Maryland, MD |
| culture (THP1)- Cell line - | | |
| THP-1 | | |
| RPMI 1640 | 11875093 | Thermofisher Scientific, Waltham, MA |
| Fetal Bovine Serum (FBS) | 10082147 | Thermofisher Scientific, Waltham, MA |
| N2 supplement (100X) | 17502048 | Thermofisher Scientific, Waltham, MA |
| Chicken serum | 16110082 | Thermofisher Scientific, Waltham, MA |
| Penicillin Streptomycin | 15140122 | Thermofisher Scientific, Waltham, MA |
| Sodium Pyruvate | 11360070 | Thermofisher Scientific, Waltham, MA |
| Duo set ELISA Human VEGF | DVE00 | RD Systems, Minneapolis, MN |
| Duo set ELISA TNF | DY210-05 | RD Systems, Minneapolis, MN |
| DuoSet ELISA Ancillary | DY008 | RD Systems, Minneapolis, MN |
| Reagent Kit 2 | | |
| IN VITRO TOXICOLOGY ASSAY | TOX1-1KT | Sigma Aldrich, St. Louis, MO |
| KIT, MTT BASED | | |
| ImmunoCult-SF | 10961 | STEMCELL Technologies, Vancouver |
| | | Canada |
| M-CSF | 78057.1 | STEMCELL Technologies, Vancouver |
| | | Canada |
| Human Interferon-gamma | 78020.1 | STEMCELL Technologies, Vancouver |
| (IFN-γ) | | Canada |
| , Lipopolysaccharide (LPS) | L7895-1MG | Sigma |
| Human IL-4 | 78045.1 | STEMCELL Technologies, Vancouver |
| | | Canada |
| Dulbecco's Phosphate- | 14040133 | Thermofisher Scientific, Waltham, MA |
| buffered Saline (DPBS) | | |
| IN VITRO TOXICOLOGY ASSAY | TOX1-1KT | Sigma Aldrich, St. Louis, MO |
| KIT, MTT BASED | | |
| Human IL-10 DuoSet ELISA | DY217B-05 | RD Systems, Minneapolis, MN |
| DuoSet ELISA Ancillary | DY008 | RD Systems, Minneapolis, MN |
| Reagent Kit 2 | | |
| Human Complement C3 | ab108823 | Abcam Inc. |
| ELISA Kit | | |
| Human Complement factor H | ab252359 | Abcam Inc. |
| ELISA Kit | | |

| THP-1 Media | Composition |
|---|---|
| Culture medium | RPMI 1640 (+L-Glutamine) |
| | 10% FBS |
| | 1% penicillin/streptomycin |
| | 1% Sodium pyruvate |
| Differentiation medium | RPMI 1640 (+L-Glutamine) |
| | 1% penicillin/streptomycin |
| | 1% Sodium pyruvate |
| | 1% N2 supplement |
| | 1% chicken serum |
| Experiment medium (serum free) | RPMI 1640 (+L-Glutamine) |
| | 1% penicillin/streptomycin |
| | 1% Sodium pyruvate |
| | 1% N2 supplement |

### Instruments Used

| Instruments | Vendor | City, State | Catalog # |
|---|---|---|---|
| Incubator | ThermoFisher Scientific, | Waltham, MA | HeraCell Vios 450i |
| Biosafety Cabinet | ThermoFisher Scientific, | Waltham, MA | 1300 Series Class II, |
| | | | Type A2 |
| Centrifuge | Eppendorf | Hamburg, Germany | 5810R |
| Microcentrifuge | Eppendrof | Hamburg, Germany | 5424R |
| Sonicator | Qsonica | Newtown, CT | Q500-110 |
| Thermocycler | ThermoFisher Scientific, | Waltham, MA | ProFlex PCR |
| qPCR | ThermoFisher Scientific, | Waltham, MA | qPCR QuantStudio 6 |
| | | | Pro |
| Western Blot imager | LI-COR | Lincoln, NE | Odyssey CLx |
| Confocal microscope | Olympus | Shinjuku City, | FLUOVIEW FV3000 |
| | | Tokyo, Japan | |
| Microplate Reader | Molecular Devices | San Jose, CA | SpectraMax i3x |
| Nanodrop | ThermoFisher Scientific, | Waltham, MA | Nanodrop One C |
| Western Blot transfer | Life Technologies | Waltham, MA | iBlot 2 |
| Gel electrophoresis | Life Technologies | Waltham, MA | PowerEase 300W |

### Abbreviations

| | |
|---|---|
| AT-007 | Ligand PSA alone |
| AT-007-NP | PSA-PLGA-nanoparticle construct |
| AMD | Age-related macular degeneration |
| ANOVA | Analysis of variance |
| AUC | Area under the curve |
| BLD | Bright light damage |
| BLI | Bio-layer interferometry |
| BSA | Bovine serum albumin |
| C3 | Complement 3 |
| CFH | Complement factor H |
| Cₘₐₓ | Maximum concentration |
| CNV | Choroidal neovascularization |
| DAMP | Damage associate molecular patterns |
| DMSO | Dimethyl sulfoxide |
| EDTA | Ethylenediaminetetraacetic acid |
| ELISA | Enzyme-linked immunosorbent assay |
| FBS | Fetal bovine serum |
| Fc | fragment crystallizable |
| fERG | Flash electroretinogram |
| ffERG | Full field electroretinogram |
| FITC | Fluorescein isothiocyanate |
| GA | Geographic atrophy |
| GAPDH | Glyceraldehyde 3-phosphate dehydrogenase |
| GLP | Good laboratory practice |
| H&E | Hematoxylin &amp; Eosin |
| Iba-1 | Ionized calcium binding adaptor molecule-1 |
| ICB | Iris-ciliary body |
| IFN-γ | Interferon-γ |
| IL | Interleukin |
| IN | Inferior nasal |
| IOP | Intraocular pressure |
| ITAM | Immunoreceptor tyrosine activating motif |
| ITIM | Immunoreceptor tyrosine inhibitory motif |
| IV | Intravenous |
| IVT | Intravitreal |
| Kdis | Dissociation constants |
| LPS | Lipopolysaccharides |
| Lux | luminance |
| M | Macrophages |
| M0 | Macrophages (non-activated) |
| M1 | Macrophages (pro-inflammatory) |
| M2 | Macrophages (anti-inflammatory) |
| M-CSF | Macrophage colony stimulating factor |
| MES | 2-Ethanesulfonic acid |
| MTT | 3-(4,5-dimethylthiazol-2-yl)-2,5-diphenyltetrazolium |
| | bromide |
| NK cells | Natural killer cells |
| NaCl | Sodium Chloride |
| NP | Nanoparticle |
| NOEL | No observable effect level |
| OD | Right eye |
| OE | Ocular examination |
| ONL | Outer nuclear layer |
| OS | Left eye |
| OU | Both eyes |
| PBS | Phosphate buffered saline |
| PBMC | Peripheral blood mononuclear cells |
| PEG | Polyethylene glycol |
| PK | Pharmacokinetics |
| PMA | Phorbol 12-myristate 12-acetate |
| PMSF | phenylmethylsulfonyl fluoride |
| PSA | Polysialic acid |
| PTP | tyrosine phosphatase |
| q | Quisque (every) |
| qRT-PCR | quantitative real-time reverse transcription polymerase |
| | chain reaction |
| RIPA | Radioimmunoprecipitation assay |
| ROS | Reactive oxygen species |
| RPE | Retinal pigment epithelium |
| RPMI | Roswell Park Memorial Institute |
| RR | Respiratory rate |
| SAMP | Self-Associated Molecular Pattems |
| SD | Standard error |
| SD-OCT | Spectral domain optical coherence tomography |
| SEM | Standard error mean |
| SHP-1 | Src homology region 2 domain-containing phosphatase-1 |
| SHP-2 | Src homology region 2 domain-containing phosphatase-2 |
| SLE | Slit-lamp examination |
| SN | Superior nasal |
| SPRR | Self-Associated Molecular Pattems |
| t_{1/2} | Half-life |
| TA | Test article |
| TBD | To be determined |
| THP-1 | THP-1 human monocyte cell line |
| Tₘₐₓ | Time to maximum concentration |
| TNF-α | Tumor necrosis factor-α |
| VEGF | Vascular endothelial growth factor |
| VIP | Volume internal projection |

### General Methods

### I. Method of Manufacturing Nanoparticles Having Polysialic Acid Ligands

### A. Preparation of Polysialic-Acid-Azide Ligands for Coupling to Nanoparticles

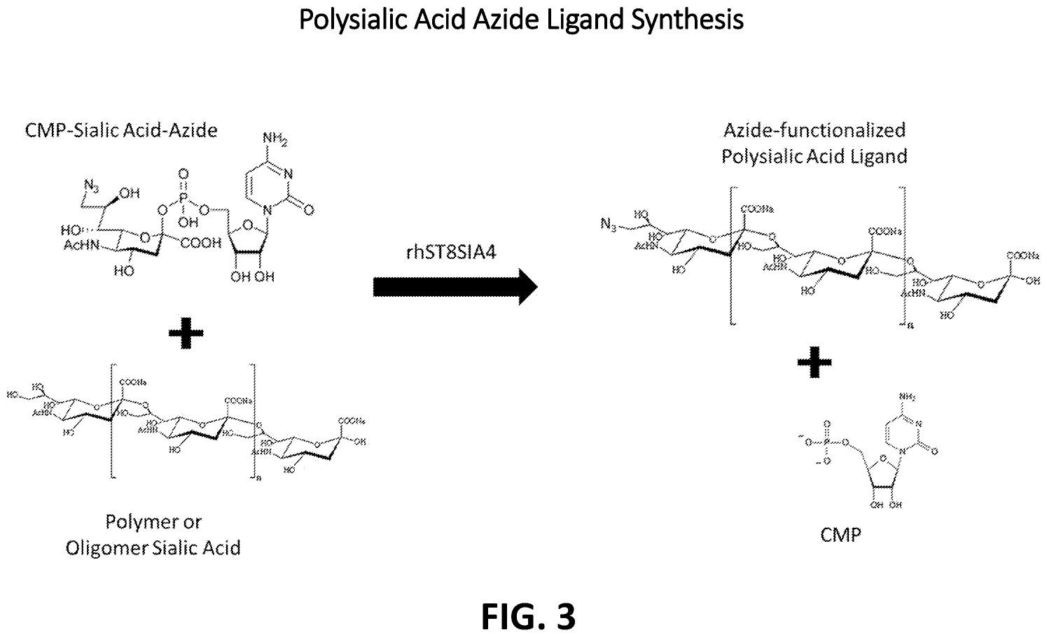

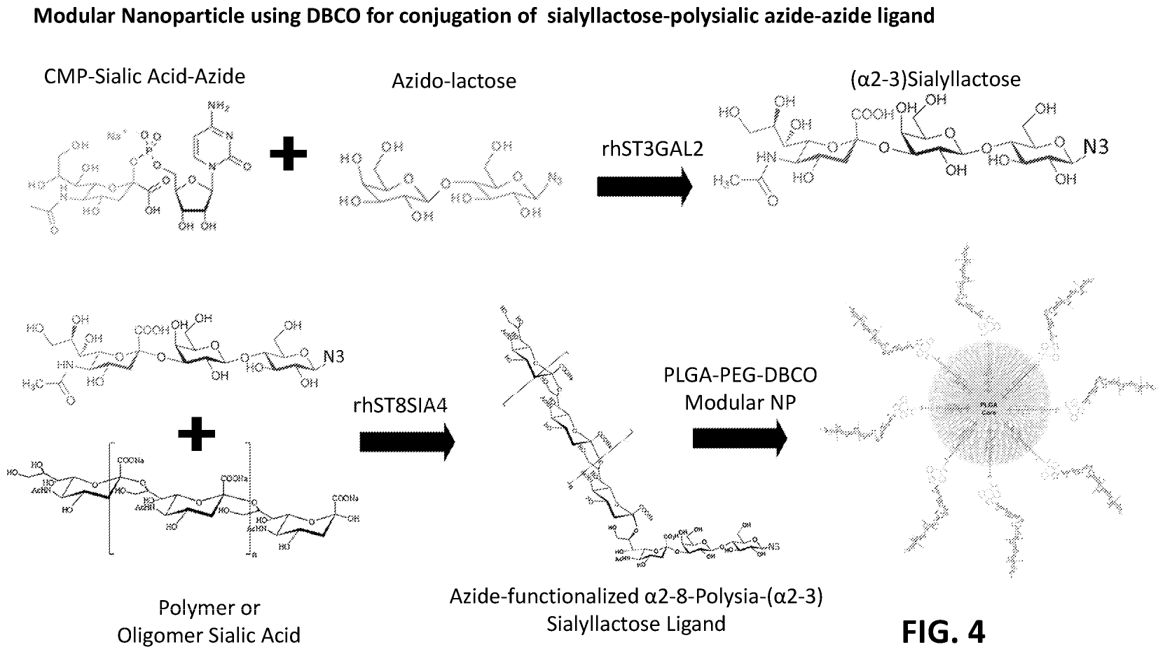

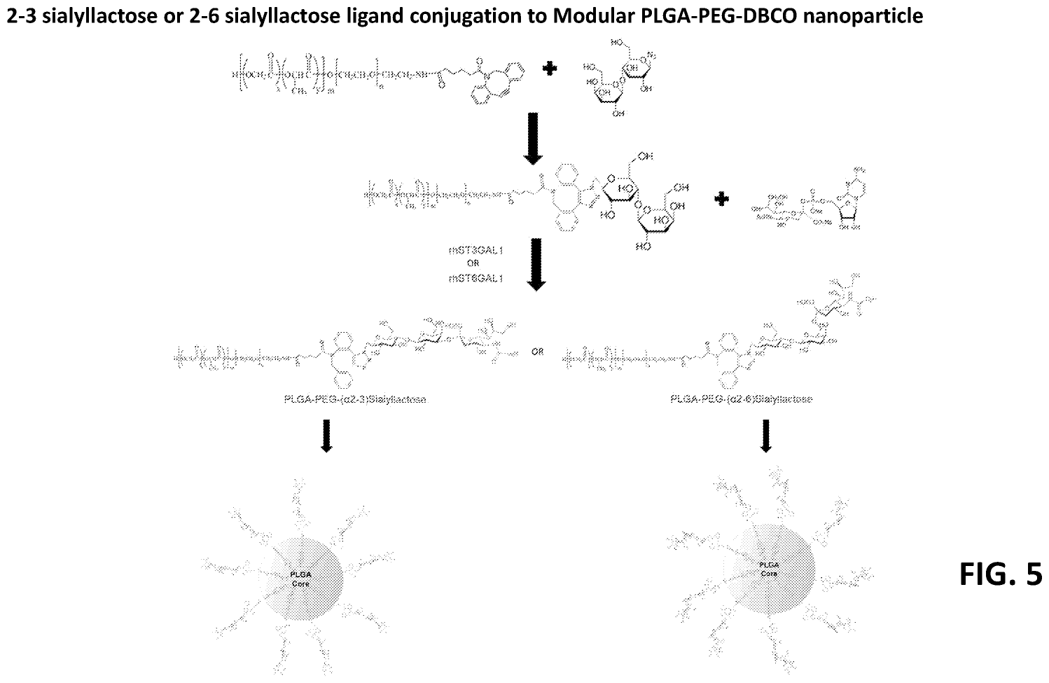

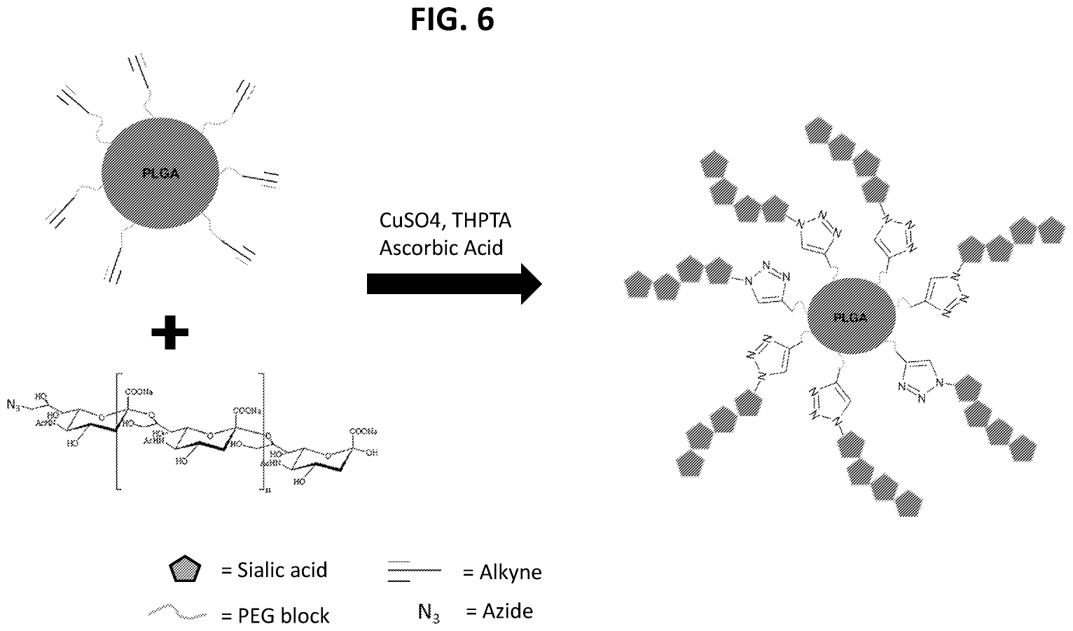

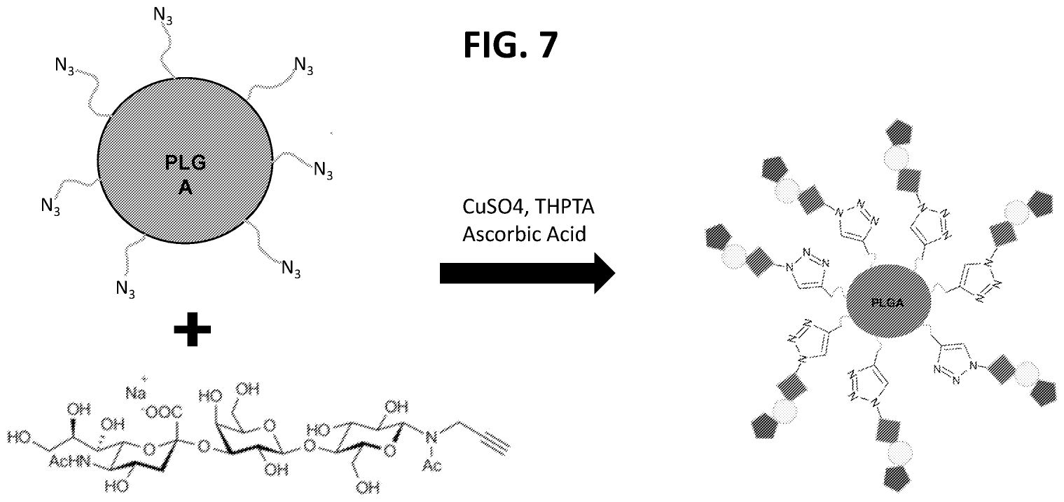

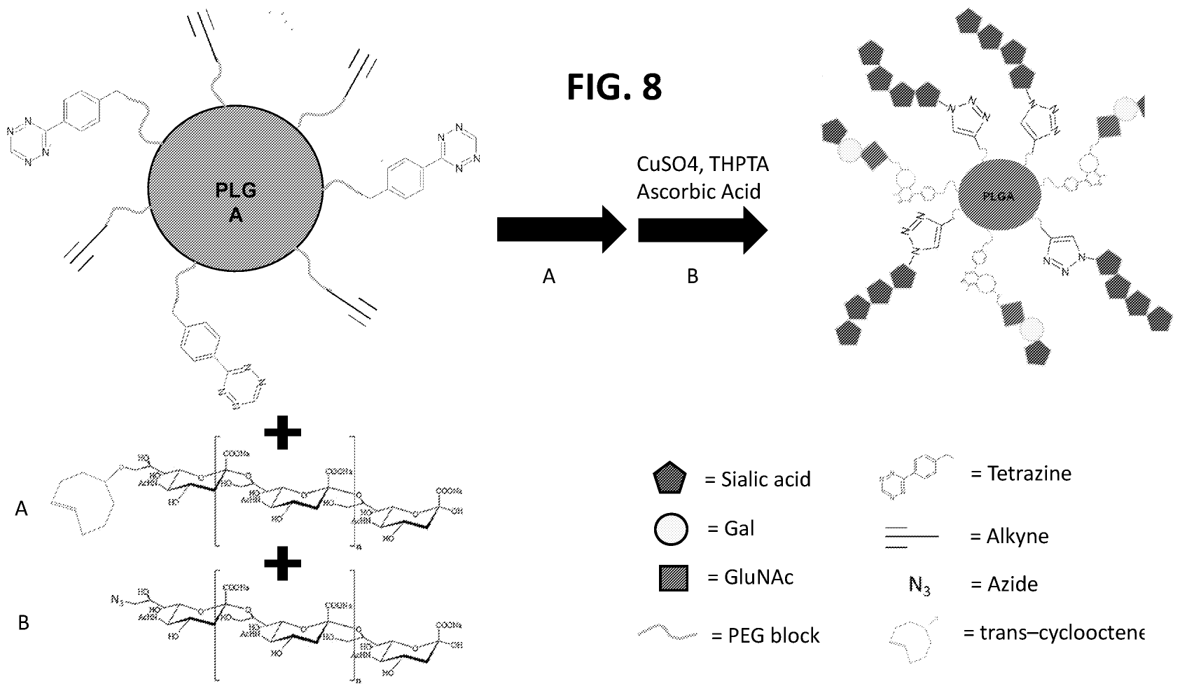

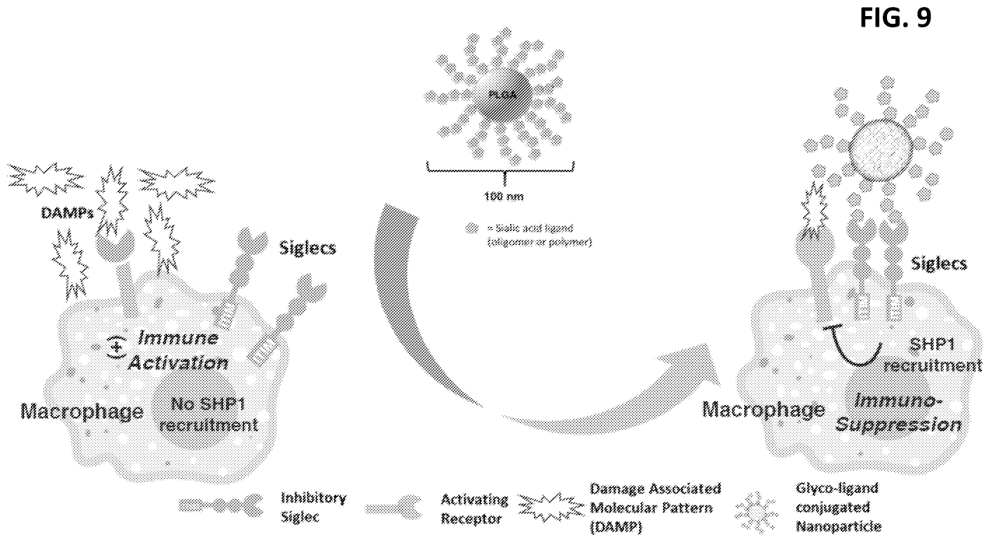

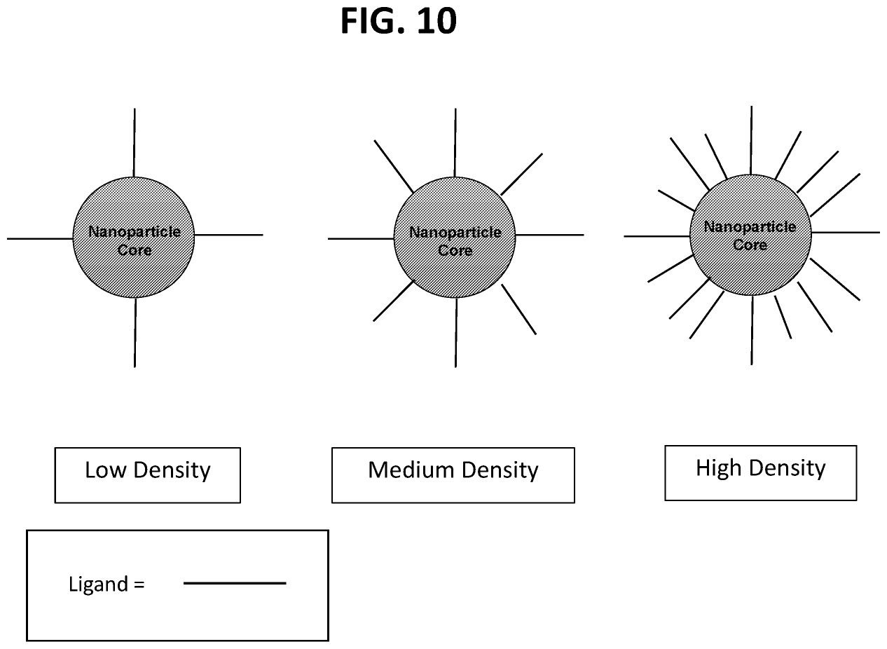

Poly sialic-acid-azide ligands (Polysia-N3 also referred to herein as PSA-N3) can be prepared by the conjugation of a terminal CMP-azido-sialic-acid moiety (CMP-Azido-sialic-acid: R&D Systems) to the C2 anomeric hydroxyl group of polysialic-acid (α2-8 Neu5Ac; colominic acid DP˜150). This conjugation was performed using purified human recombinant sialyltransferase ST8SIA4 (CMP-N-acetylneuraminate-poly-alpha-2,8-sialyltransferase; R&D SystemsThe subsequent polysia-N3 can provide a single click chemistry-activated azide functional group per polysialic-acid polymer molecule, which can therefore allow highly specific conjugation to the PLGA-alkyne nanoparticle surface. The use of targeted click chemistry can yield the optimal 3D orientation of the polysia ligands for presentation to Siglec-expressing cells and provides a significantly superior synthesis route compared to carbodiimide chemistry (i.e., EDC-NHS). FIG. 3 depicts a synthetic scheme for the preparation of Polysialic-acid-azide ligands (Polysia-N3).

### 1B. Preparation of DBCO-Functionalized PLGA Nanoparticles

DBCO-functionalized PLGA nanoparticles that form the core nanoparticle onto which the polysialic acid ligands are attached can be prepared via an emulsion method. Core PLGA nanoparticles can be prepared by blending Poly(lactide-co-glycolide)-b-Poly(ethylene glycol)-Carboxylic acid (PLGA-PEG-COOH; Nanosoft Polymers; MW˜10,000:5,000 Da) and poly(lactide-co-glycolide)-b-poly(ethylene glycol)-azide (PLGA-PEG—dibenzo-bicyclo-octyne (DBCO): Nanosoft Polymers: MW˜10,000:5,000 Da) at a 75:25 (w/w) ratio of (PLGA-H):(PLGA-PEG-COOH+PLGA-PEG-DBCO). The 75:25 ratio can also be changed to 50:50 or 25:75. The ratio of PLGA-PEG-COOH:PLGA-PEG-DBCO can be varied to increase or decrease the concentration of DBCO groups on the nanoparticle surface. The selected ratio provides a reasonably high density of azide functional groups on the nanoparticle surface, while allowing sufficient space between the functional groups to permit efficient conjugation of the polymer ligands.

To introduce a fluorescence moiety into the nanoparticles, a polymer, fluorescein-PLA or fluorescein-PLGA was used. The total fraction of the fluorescein polymer in the total polymer used for preparing the nanoparticles was 1% by weight. Blank nanoparticles were used as controls in subsequent experiments. These blank nanoparticles were from the same batch of nanoparticles as described above but were not subjected to any PSA ligands conjugation.

### 1C. Method to Quantify Free DBCO Groups on the Nanoparticle Surface

A spectrofluorometric assay method can be used for quantifying free DBCO groups on the nanoparticle surface. The method involves conjugating an azide-functionalized fluorophore (e.g., Cy3-N3) to the prepared nanoparticles with DBCO groups on the surface. After the conjugation, the nanoparticle solution is washed to remove unreacted material and the fluorescence of the solution is determined. The concentration of available DBCO groups on the nanoparticle surface is determined from a standard curve generated using Cy-3.

### 1D-1. Conjugation of PSA-N3 Ligand to DBCO-Functionalized Nanoparticles

Purified PSA-N3 ligand can be conjugated to the surface of purified PLGA-DBCO nanoparticles via a SPAAC copper-free click chemistry protocol. Briefly, the nanoparticles with DBCO are incubated with the PSA-azide at 4° C. or room temperature or at 37° C. overnight. Upon completion of the conjugation reaction, the nanoparticles are washed using TFF to remove unreacted components, and the MES buffer is replaced with PBS or an appropriate isotonic solution such as 10% Sucrose. The nanoparticle solution is then sterile filtered via a 0.2 μm filter. The size of the final PSA-conjugated PLGA nanoparticles (PSA-PLGA NP) is measured by dynamic light scattering (DLS). The PSA-PLGA nanoparticles are stabilized due to the highly negative charge of the sialic-acid moieties and the presence of PEG-COOH chains, which can be confirmed by zeta potential measurement. The final polysia-PLGA nanoparticle product can be stored in PBS at 1 mg/ml concentration at 4 degrees C. or in 10% sucrose at −20 degrees C. for subsequent use. The final product, PSA-PLGA nanoparticles, can be characterized for size (DLS) and surface charge (zeta potential measurement).

### 1D-2. Conjugation of PSA-N3 Ligand to DBCO-Functionalized Nanoparticles

The fraction of DBCO-PEG-PLGA polymer that was used in the preparation of the nanoparticles was used to determine the amount of this polymer present per mg of total solids of the nanoparticles. Purified PSA-N3 ligand was conjugated to the surface of purified PLGA-DBCO nanoparticles via a SPAAC copper-free click chemistry protocol. Briefly, the nanoparticles with DBCO were incubated with the PSA-azide at 4° C. or room temperature or at 37° C. overnight. The ratio of PSA-azide to the DBCO-PEG-PLGA present in the nanoparticles ranged from 0.75:1.0 to 1:1 by weight. Upon completion of the conjugation reaction, the nanoparticles were washed using TFF to remove unreacted components, and the MES buffer was replaced with PBS or an appropriate isotonic solution such as 10% Sucrose. The nanoparticle solution was then sterile filtered via a 0.2 μm filter. The size of the final PSA-conjugated PLGA nanoparticles (PSA-PLGA NP) was measured by dynamic light scattering (DLS). The PSA-PLGA nanoparticles are stabilized due to the highly negative charge of the sialic-acid moieties and the presence of PEG-COOH chains, which can be confirmed by zeta potential measurement. The final PSA-PLGA nanoparticle product was stored in PBS at 4 degrees C. or in 10% sucrose at −20 degrees C. for subsequent use. The final product, PSA-PLGA nanoparticles, was characterized for size (DLS) and surface charge (zeta potential measurement).

### 1E. Determining the Conjugation Efficiency of Polysialic Acid (PM)-Containing Ligand on Nanoparticles

Determining density of PSA-acid ligands conjugated to the nanoparticle surface and the click chemistry reaction conjugation efficiency are key to optimizing the nanoparticles described herein. The density of alkyne surface functional groups will tend to dictate the maximum density of the polymer ligands that can be conjugated to the surface of the nanoparticles. The actual density of conjugated polysia ligands will also depend on the Degree of Polymerization (DP; number of sialic-acid units) of the poly sialic acid (polysia). However, steric effects and repulsive forces of the negative charged sialic-acids could prevent complete conjugation of surface functional groups. The density of the nanoparticle surface alkyne functional groups prior to conjugation will need to be determined, which can be achieved via fluorophore-azide conjugation and quantified from a standard curve using a spectrofluorometer. Furthermore, the alkyne functional group density can be validated by X-ray photoelectron spectroscopy (XPS) mapping, which may potentially be more accurate. By quantifying the density of alkyne groups before ligand conjugation, and subsequently quantifying the amount of unconjugated ligand post-conjugation step, the conjugation efficiency of the click chemistry reaction for the PLGA polysialic-acid system can be calculated.

Additionally, the density of ligand conjugation on the surface of the nanoparticles will need to be determined. The ligand density can be determined by quantifying the total surface of the unconjugated nanoparticles on a per mg basis via nitrogen desorption method, given as m² or nm². After conjugating the nanoparticles with the polysialic-acid ligands the mass of polysialic-acid can be quantified by differential scanning calorimetry (DSC) and/or thermogravimetric analysis (TGA). Converting mass of polysialic acid to number of molecules (using the average molecular weight of the conjugated polysialic-acid), and then dividing the number of molecules by the total surface of area of the given mass of the sample of nanoparticles, yields the polysialic-acid ligand density in units of molecules/nm². In certain embodiments, the density of the polysialic-acid ligands on the particles is from 0.1 molecule/nm² to 5 molecules/nm².

### 1F. Determining the Concentration of Polysialic Acid (PSA)-Containing Ligand on Nanoparticles

The concentration of Polysia that is conjugated to the surface of the nanoparticles was determined by available Sialic acid assay kits. Sialic acid assay kit measures free sialic acid (mainly N-acetylneuraminic acid or NANA) in a variety of samples. The detection is based in an enzyme coupled reaction in which oxidation of free sialic acid creates an intermediate that reacts stoichiometrically with the probe to generate a product that can detected by absorbance (OD=570 nm) or fluorescence (Ex/Em=535/587 nm). The kit measures sialic acid in the linear range of 0.1 to 10 nmol with a detection sensitivity ˜1 μM concentration. (Ref: https://www.abcamcom/sialic-acid-nana-assaykit-ab83375.html). The concentration of PSA was normalized to the total weight of the nanoparticles or nmol of PSA/mg of nanoparticles.

### II. Example 1: Production of Exemplary Nanoparticles

Nine batches of nanoparticles were prepared. Table 2 identifies each prepared batch and provides details on the production of each batch.

With the exception of batch AT-007 NP06, nanoparticles were prepared by the emulsion method. A representative example of nanoparticle production included the following. PLGA(10 k)-PEG(5 k)—COOH and PLGA(10 k)-PEG(5 k)-DBCO were weighed at a weight ratio of 3:1 and dissolved in mixture of organic solvents. The concentration of the polymer was 37 mg/mL in organic solvent (59.5% ethyl acetate and 40.5% benzyl alcohol). 10 mL of the polymer solution was added to 40 mL of an aqueous phase consisting of 0 or 0.1% Tween 80 in water and homogenized using a Rotastator to form a coarse emulsion. The coarse emulsion was further homogenized to a fine nanoemulsion using a microfluidizer with 3 passes. The nanoemulsion was then quenched by addition to 450 mL of cold water to harden the nanoparticles. The quenched nanoparticle solution was then concentrated and washed by tangential flow filtration to remove the organic solvent. The concentration of polymeric nanoparticles was determined by evaporating water from a known volume of nanoparticle solution. The water in nanoparticle solution was then replaced with 50 mM 2-(N-morpholino)ethanesulfonic acid (MES) hydrate buffer at pH 5 using TFF or by addition of a concentrated MES buffer solution. The nanoparticle solution was then prepared for conjugation to sialic acid (PSA-N3) ligand.

A detailed description of the Nanoparticle Compositions is provided in Table 3. The nanoparticles were characterized and the results are shown in Table 4.

TABLE 3
Steps of Nanoparticle Fabrication
Core Nanoparticle Fabrication

| Step | Description |
|---|---|
| 1 | Prepare polymer solution (~37 mg/mL) in a cosolvent system |
| | of ethyl acetate/benzyl alcohol (79%/21% w/w) through |
| | overnight dissolution. |
| 2 | Prepare 7.5% w/w aqueous solution of ethyl acetate. |
| 3 | Syringe filter polymer solution using a 0.2- μm PTFE filter. |
| 4 | Homogenize polymer solution in aqueous phase via rotor stator |
| | (10,000 rpm) for one minute. |
| 5 | Process homogenized solution via a standard five-stroke |
| | procedure with the Microfluidizer operating at 10,000 psi. |
| 6 | Quench the sample in water. |
| 7 | Three hours post-quench, purify and collect the sample via |
| | tangential flow filtration. |
| 8 | Concentrate the sample to its desired volume and determine |
| | solids content. |

TABLE 4
Compositions of Nanoparticle Batches

| Batch # | | | | |
|---|---|---|---|---|
| Original/ | | | | Ratio of |
| Updated (if | | | | Aqueous/ |
| different or | | | Formulation | Organic |
| available) | Description | Polymers | Composition | phase |
| Batch A 100052- | PSA-NPs in PBS | PLGA(10 k)-PEG-(5 k)-COOH | 37 mg/mL polymer in (79% | 9:1 |
| 21/AT-007 NP01 | and 10% Sucrose | (75%)/PLGA(10 k)- | EtAc/21% BzOH)/WFI | |
| | | PEG(5 k)-DBCO (25%) | (7.5% EtOH) | |
| Batch A 100052-22 | Blank NPs in PBS | PLGA(10 k)-PEG-(5 k)-COOH | 37 mg/mL polymer in (79% | 9:1 |
| | and 10% Sucrose | (75%)/PLGA(10 k)- | EtAc/21% BzOH)/WFI | |
| | | PEG(5 k)-DBCO (25%) | (7.5% EtOH) | |
| Batch B 1000154- | PSA NPs in | PLGA(10 k)-PEG-(5 k)-COOH | 37 mg/mL polymer in (79% | 9:1 |
| 1/AT-007 NP02 | 10% Sucrose | (75%)/PLGA(10 k)- | EtAc/21% BzOH)/WFI | |
| | | PEG(5 k)-DBCO (25%) | (7.5% EtOH) | |
| Batch B 1000154-2 | Blank NPs in | PLGA(10 k)-PEG-(5 k)-COOH | 37 mg/mL polymer in (79% | 9:1 |
| | 10% Sucrose | (75%)/PLGA(10 k)- | EtAc/21% BzOH)/WFI | |
| | | PEG(5 k)-DBCO (25%) | (7.5% EtOH) | |
| Batch C 1000182- | PSA-FITC NPs in | PLGA(10 k)-PEG-(5 k)-COOH | 37 mg/mL polymer in (79% | 9:1 |
| 1/AT-007 NP03 | 10% Sucrose | (89%)/PLA-FITC (1%)/ | EtAc/21% BzOH)/WFI | |
| | | PLGA(10 k)-PEG(5 k)-DBCO | (7.5% EtOH) | |
| | | (10%) | | |
| Batch C 1000182-2 | Blank FITC NPs | PLGA(10 k)-PEG-(5 k)-COOH | 37 mg/mL polymer in (79% | 9:1 |
| | in 10% Sucrose | (89%)/PLA-FITC (1%)/ | EtAc/21% BzOH)/WFI | |
| | | PLGA(10 k)-PEG(5 k)-DBCO | (7.5% EtOH) | |
| | | (10%) | | |
| In vivo 100205-1/ | PSA-NPs in | PLGA(10 k)-PEG-(5 k)-COOH | 37 mg/mL polymer in (79% | 9:1 |
| AT-007 NP04 | 10% Sucrose | (90%)/PLGA(10 k)-PEG(5 k)- | EtAc/21% BzOH)/WFI | |
| | | DBCO (10%) | (7.5% EtOH) | |
| In vivo 100205-2 | Blank NPs in | PLGA(10 k)-PEG-(5 k)-COOH | 37 mg/mL polymer in (79% | 9:1 |
| | 10% Sucrose | (90%)/PLGA(10 k)-PEG(5 k)- | EtAc/21% BzOH)/WFI | |
| | | DBCO (10%) | (7.5% EtOH) | |
| —/AT-007 NP06 | PSA-NPs in 10% | PLGA(10 k)-PEG-(5 k)-COOH | 100 mg/mL polymer in 48% | 9:1 |
| | sucrose | (90%)/PLGA(10 k)-PEG(5 k)- | ethyl acetate/32% benzyl | |
| | | DBCO (5%) | alcohol/20% DMSO/WFI | |
| | | | (7.5% EtOH) | |

Notes:
1-The key properties of the PLGA used in the nanoparticles can be determined in accordance with procedures found in “Graner, J. et al., International Journal of Pharmaceutics; 495 (2015) 87-92.
2-All formulations except NP06 were made according to Example 1 (I); AT-007 NP06 was made according to Example 4 (V).

### III. Example 2: Enzymatic Addition of CMP-Azido-Sialic Acid to Colominic Acid (PSA-N3)

TABLE 5
Description of Process to Prepare PSA-N3
Enzymatic Addition of CMP-Azido-Sialic Acid to Colominic Acid

| Step | Description |
|---|---|
| 1 | Prepare a 1.9 mg/mL solution of low endotoxin Colominic acid in 1x MES/MnCl₂ Buffer (100 mM MES |
| | Buffer; 10 mM MnCl₂ Buffer; pH = 7.0) |
| 2 | Thaw enzyme (ST8SIA4 protein) and donor substrate (CMP-Azido-Sialic acid) on ice. |
| 3 | Add required volume of donor substrate to the Colominic acid solution to achieve a 2-fold molar excess |
| | of donor with respect to the Colominic acid. Gently vortex the solution. |
| 4 | Add required volume of enzyme to achieve a 200-fold molar excess of Colominic acid to enzyme. Gently |
| | vortex the solution. |
| 5 | Incubate the reaction mixture at 37° C. on an orbital shaker for 20 hours. |
| 6 | Following the incubation period, store the sample at 4° C. until purification and buffer exchange via ultra- |
| | filtration. |
| 7 | Quantify the enzymatic activity of ST8SIA4 following the manufacturer's protocol described in the |
| | sialyltransferase activity assay kit (R&D Systems). |
| 8 | Prepare for Colominic acid purification by equilibrating an Amicon 15 mL (MWCO = 10 k) filter unit by |
| | adding 12 mL of DI water and centrifuging at 5,000 rcf for 30 minutes. |
| 9 | Discard the filtrate and retentate from the filter unit. |
| 10 | Load sample (Maximum Volume: 12 mL) and centrifuge at 5,000 rcf for 30 minutes at 4° C. |
| 11 | Following centrifugation, discard the filtrate. Sample volume (retentate volume) is ~1 mL. |
| 12 | Dilute sample with 11 mL of 1x PBS and centrifuge at 5,000 rcf for 30 minutes at 4° C. |
| 13 | Following centrifugation, discard the filtrate. Sample volume (retentate volume) is ~1 mL. |
| 14 | Repeat Step 12 and 13 two additional times. |
| 15 | Transfer retentate to a 15 ml conical tube. Note: The presence of precipitated ST8SIA4 protein may be |
| | observed as white precipitate suspended in the sample. |
| 16 | Rinse the filter unit membrane with two 1 ml aliquots of PBS. Transfer rinsate to the 15 ml conical |
| | tube. |
| 17 | Centrifuge the recovered sample at 5,000 rcf for 5 minutes at 4° C. |
| 18 | Using a 1 mL pipette, carefully transfer the supernatant (i.e. the PSA-azide solution) to a 50 ml conical |
| | tube. Discard the remaining protein pellet. |
| 19 | Collect an aliquot to determine the PSA-azide concentration via NANA assay (Section 7). |
| 20 | Store the PSA-azide solution at 4° C. until further use. |

### IV. Example 3: PSA-Azide Conjuration to Core DBCO Nanoparticle Surface Via SPAAC Chemistry

Purified PSA-N3 ligand prepared as described above was conjugated to the surface of purified PLGA-DBCO nanoparticles via a SPAAC copper-free click chemistry protocol. Briefly, the nanoparticles with DBCO were incubated with the PSA-azide at 4° C. or room temperature or at 37° C. overnight. Upon completion of the conjugation reaction, the nanoparticles were washed using TFF to remove unreacted components, and the MES buffer was replaced with PBS or an appropriate isotonic solution such as 10% Sucrose. The nanoparticle solution was then sterile filtered via a 0.2 μm filter.

TABLE 6
Details of the PSA-Azide Conjugation to the Core DBCO Nanoparticles via SPAAC Chemistry
PSA-Azide Conjugation to Core DBCO Nanoparticle Surface via SPAAC Chemistry

| Step | Description |
|---|---|
| 1 | Based on the concentration of PSA-azide determined via the NANA assay and the mass of the PSA-azide |
| | solution, calculate the mass of PSA-azide available for conjugation. |
| 2 | Utilizing the molecular weight of Colominic acid (as reported in the CoA) and mass of PSA-azide |
| | quantified in Step 1, calculate the moles of PSA-azide available for conjugation. |
| 3 | Utilizing the moles of azide quantified in Step 2, calculate the mass of core DBCO nanoparticles required |
| | to achieve a 1:1 molar ratio of azide to DBCO. |
| 4 | Utilizing the mass of core DBCO nanoparticles quantified in Step 3, calculate the volume of the core |
| | DBCO nanoparticle solution required to achieve a 1:1 molar ratio of azide to DBCO groups. |
| 5 | Equilibrate core DBCO nanoparticle and PSA-azide solutions to room temperature. |
| 6 | Add required volume of DBCO nanoparticle solution to the PSA-azide solution contained in the 50 mL |
| | conical tube. Vortex solution to mix. |
| 7 | Incubate the reaction mixture at 25° C. on an orbital shaker for 18 hours. |

TABLE 7
Post-Conjugation Processing of Nanoparticles
Post-Conjugation Processing of Nanoparticles

| Step | Description |
|---|---|
| 1 | Upon completion of the incubation process, purify and |
| | concentrate the nanoparticle suspension via tangential |
| | flow filtration (TFF). |
| 2 | To reduce the presence of free ligand, complete four (4) |
| | wash steps with a 10x volume of water for injection (WFI). |
| 3 | Concentrate the nanoparticle suspension to its target volume. |
| 4 | Assess solids content of the nanoparticle suspension. |
| 5 | Following the removal of free PSA-azide and nanoparticle |
| | concentration via TFF, syringe filter the suspension using |
| | a 0.22- μm filter. |
| 6 | Assess solids content of the nanoparticle suspension, |
| 7 | Adjust concentration using WFI as needed. |
| 8 | Mass out sucrose. |
| 9 | Add sucrose to nanoparticle suspension and vortex until dissolved. |
| 10 | Store suspension at 4° C. until use. |

TABLE 8
Details of Nanoparticles Post-Conjugation

| | | | | | | | Ligand | | | | Mol. |
|---|---|---|---|---|---|---|---|---|---|---|---|
| | PSA- | | Post- | | Total | | Density | Total | | Particle | weight |
| | Azide:Polymer | Pre-filter | filter | Zeta | Solids | PSA | ug | Polymer | % | Concen- | of |
| Batch | ratio | Diameter | Diameter | potential | Conc | Conc | (Lig)/mg | weight, | Conjug. | tration, | ligand, |
| # | (mg/mg) | (nm)/PDI | (nm)/PDI | (mV) | (mg/mL) | (mg/mL) | (solid) | mg | Efficiency | #/ml | g/mol |
| Batch A | 0.75:1 | 134.7 | N/A | −37.9 | 1.6 | 0.4 | 250 | 25 | 33 | 9.62267E+11 | 60,000 |
| 100052- | | (0.33) | | | | | | | | | |
| 21/AT-007 | | | | | | | | | | | |
| NP01 | | | | | | | | | | | |
| Batch A | N/A | 134.6 | N/A | −20.9 | 1.6 | 0 | 0 | | | | |
| 100052-22 | | (0.45) | | | | | | | | | |
| Batch 8 | 1:1 | 121.0 | 120.6 | −39.7 | 3.4 | 1.4 | 412 | 25 | 66 | 2.84915E+12 | 60,000 |
| 1000154- | | (0.133) | (0.12) | | | | | | | | |
| 1/AT-007 | | | | | | | | | | | |
| NP02 | | | | | | | | | | | |
| Batch B | N/A | 108.2 | 106.9 | −33.6N | 5.6 | 0 | 0 | | | | |
| 1000154-2 | | (0.087) | (0.17) | | | | | | | | |
| Batch C | 1:1 | | 101.3 | −44 | 6.9 | 1.36 | 197 | 10 | 61 | 9.75661E+12 | 60,000 |
| 1000182- | | | (0.06) | | | | | | | | |
| 1/AT-007 | | | | | | | | | | | |
| NP03 | | | | | | | | | | | |
| Batch C | N/A | | 105.7 | −27 | 6.9 | 0 | 0 | | | | |
| 1000182-2 | | | (0.07) | | | | | | | | |
| In vivo | 1:1 | | 95.4 | −46 | 16.8 | 2.6 | 155 | 10 | 45 | 2.84408E+13 | 60,000 |
| 100205- | | | (0.08) | | | | | | | | |
| 1/AT-0007 | | | | | | | | | | | |
| NP04 | | | | | | | | | | | |
| In vivo | N/A | | 89.4 | −23 | 16.8 | 0 | 0 | | | | |
| 100205-2/ | | | (0.11) | | | | | | | | |
| AT-007- | N/A | N/A | 139 | N/A | 5.81 | 0.137 | 24 | 5 | 74 | 3.17987^{E}+12 | 25,000 |
| NP06 | | | (0.19) | | | | | | | | |

1-The size of the nanoparticles is determined in accordance with the following procedure: Hackley V A, Clogston J D, NIST-NCL Joint Assay Protocol, PCC-1: Measuring the Size of Nanoparticles in Aqueous Media Using Batch-Mode Dynamic Light Scattering. https://ncl.cancer.gov/resources/assay-cascade-protocols DOI: 10.17917/3F5S-6728
2-The quantitation of sialic acid in the Poly sialic acid nanoparticles was determined using the NANA Assay described in Method 1F above and detailed below.
3-The Zeta Potential was determined in accordance with the following method: Clogston J D, Vermilya A, NCL Method PCC-2: Measuring Zeta Potential of Nanoparticles. https://ncl.cancer.gov/resources/assay-cascade-protocols DOI: 10.17917/ZB8P-FB40

Specifically, the sialic acid content in PSA-NPs was determined by NANA Assay (NANA Assay Kit, ab83375 from Abcam). The NANA Assay is a simple and convenient method to measure free sialic acid (N-acetylneuraminic acid or NANA). The detection is based on enzymatic coupling reaction in which oxidation of free sialic acid creates an intermediate product that can be detected by absorbance (OD=570 nm) or fluorescence (Ex/Em=535/587 nm). The fluorescence detection was chosen to quantitate sialic acid content in nanoparticle formulations to ensure higher sensitivity. A step-by-step description of the NANA assay is provided in the table below.

NANA Assay Procedure

| Step | Description |
|---|---|
| 1 | Prepare a fresh sialic acid standard curve (in duplicate) |
| | ranging from 0-1 nmol. |
| 2 | Dissolve PolySia nanoparticle samples in 2M acetic acid |
| | and heat to 80° C. for 3 hours. |
| 3 | Add 2M sodium hydroxide to neutralize the solution. |
| 4 | Store the neutralized solution at 4° C. until use. |
| 5 | Follow the manufacturer's protocol to quantify the sialic |
| | acid concentration in each sample. (relevant excerpts |
| | provided in the Appendix) |
| 6 | Calculate the raw sialic acid concentration using Equation 7-1. |
| 7 | Adjust concentration to account for degree of hydrolysis by |
| | normalizing to a concurrently run internal control. |

The following equation was used to calculate the concentration of sialic acid (nmol/μL) in the test samples.

$${C}_{SA} = \frac{A}{V} \times D$$

Where:
   C_{SA}=Concentration of Sialic Acid (nmol/μL)
   A=Amount of Sialic Acid in the sample well (nmol)
   V=Volume of Sample in the Well (μL)
   D=Sample Dilution Factor

PSA-PLGA batches used for in vitro Studies

| | | | | PSA-PLGA |
|---|---|---|---|---|
| Cell Type | Assay | Study design | Results | batch No. |
| THP-1 derived | MTT toxicity - TOX1- | | PSA-PLGA (50-750 μg/mL) | Batch B, C |
| macrophages and | 1KT | | did not show any | and D |
| Macrophages (M1 and | | | cytotoxicity in THP-1 or | |
| M2) derived from | | | PBMC derived | |
| normal PBMC | | | macrophages as | |
| | | | demonstrated by MTT | |
| | | | assay. | |
| THP-1 derived | ELISA for TNF-α and | | PSA-PLGA (75 μg/mL) | Batch B, C |
| macrophages | VEGF | | inhibited TNF-α and | and D |
| | | | VEGF production | |
| | | | exhibited a 2-fold | |
| | | | decrease after LPS | |
| | | | activation | |
| Macrophages (M1 and | ELISA for IL-10, C3, | | PSA-PLGA (75 μg/mL) | Batch B, C |
| M2) derived from | and CFH | | increased IL-10 by 8 folds | and D |
| normal PBMC | | | in activated macrophages. | |
| | | | PSA-PLGA (75 μg/mL) | |
| | | | increased CFH production | |
| | | | by 2 folds while | |
| | | | decreasing C3 | |
| | | | concentration by 3-folds | |
| | | | in activated macrophages | |
| | Binding Assay | Octet Assay | PSA-PLGA exhibited | Batch B |
| | | | significant and specific | |
| | | | binding to Siglecs 11, 9, 7, 5 | |
| | | | compared to blank- PLGA | |

### V. Example 4: Additional Production of Exemplary Nanoparticles

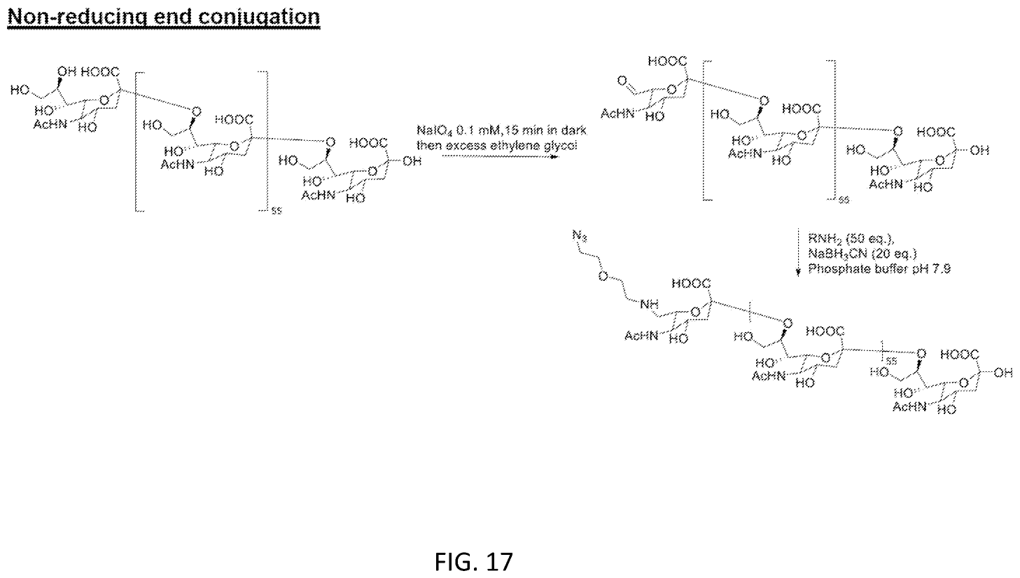

Nanoparticles were prepared by the emulsion method. A representative example of nanoparticle production is described below. The non-reducing end of PSA (colominic acid from Carbosynth) was modified with an azide group (see FIG. 17). 10 mg of the PSA with an azide group on the non-reducing end was dissolved in 0.6 mL of DMSO. The PSA-azide solution was added to 15 mg of PLGA (10 k)-PEG (5 k)-DBCO (dissolved in an organic solvent mixture of ethyl acetate:benzyl alcohol). The mixture was stirred and mixed overnight to allow for conjugation of the PSA-azide to the PLGA-PEG-DBCO via the azide-DBCO click chemistry coupling. After mixing overnight. 285 PLGA (10 k)-PEG(5 k)—COOH was added to this reaction mixture and the solution was mixed/stirred till it was visually clear.

The final volume of the organic solvents was 3.03 mL with 2.43 mL of ethyl acetate:benzyl alcohol at a volume ratio of 60:40, and 0.6 mL of DMSO. In order to introduce a fluorescence moiety into the nanoparticles, a polymer, fluorescein-PLGA was used. The total fraction of the fluorescein polymer in the total polymer used for preparing the nanoparticles was 1% by weight, i.e., 3 mg of PLGA-fluorescein was also added to the polymer solution.

This polymer solution was added to 27 mL of cold water (saturated with ethyl acetate) and homogenized using a IKA T-18 Rotastator to form a coarse emulsion. The coarse emulsion was further homogenized to a fine nanoemulsion using a microfluidizer (Microfluidics LM10) with 3 passes. The nanoemulsion was then quenched by addition to 270 mL of cold water to harden the nanoparticles. The quenched nanoparticle solution was then concentrated and washed using cold water by tangential flow filtration (KR2i TFF, Repligen) to remove the organic solvents and unreacted PSA-azide. The concentration of polymeric nanoparticles was determined by evaporating water from a known volume of nanoparticle solution. Sucrose was added to the to the nanoparticle solution at 10% wt/wt and filtered using a 0.2 μm Millipore syringe filter. The nanoparticle solution was frozen at −20° C.

The size of the nanoparticles was measured using Dynamic Light Scattering using a Malvern Zetasizer. Dynamic light scattering techniques use the constant random thermal motion of particles and molecules called Brownian motion to measure the size. The particles diffuse at a speed related to their size, smaller particles diffusing faster than larger particles. The diffusion speed is measured from the speckle pattern produced by illuminating the particles with a laser. The fluctuations in the scattering intensity at a specific angle is detected using a sensitive photodiode detector. The intensity changes are analysed with a digital autocorrelator to generates a correlation function. This curve is analysed to give the size and the size distribution of the particles. The nanoparticles were diluted to a concentration of approximately 0.1-1.0 mg/mL using clean water and measured in the Zetasizer. Each value generated was an average of 3 readings. NANA Assay was used to determine the concentration of PSA ligands. The colominic acid or PSA chains were hydrolysed into sialic acid monomers using either acid hydrolysis or sialidase enzymes. The free sialic acid was released and hydrolysed from free PSA or PSA conjugated to the nanoparticles using this treatment. Sialic Acid Assay Kit (ab83375) was used for measuring the free Sialic Acid (mainly N-acetylneuraminic acid or NANA) from the PSA. The detection is based in an enzyme coupled reaction in which oxidation of free sialic acid creates an intermediate that reacts stoichiometrically with the sialic acid probe to generate a product that can detected by absorbance (OD=570 nm) or fluorescence (Ex/Em=535/587 nm). The final concentration of sialic acid detected is determined by using a calibration curve from sialic acid standard.

The total solids concentration of the nanoparticles was determined by weighing a known amount of the nanoparticle solution in a 1.5 mL microcentrifuge tube. The solution was then frozen and lyophilized. The weight of the lyophilized nanoparticle powder was normalized to the weight of the solution to give the wt/wt concentration of the nanoparticles in water. The nanoparticle formulations that were produced were characterized by calculating the ug of PSA/mg of total solids. For the nanoparticles prepared by the procedure described in the above example 2, the conjugation efficiency was determined as follows. The ratio of PSA and total solids (polymers+PSA) used to make the organic phase was calculated. The final ratio of PSA to the total solids was determined using the NANA assay and lyophilization of the nanoparticle solution (described above). The overall conjugation efficiency was calculated as

$$Conjugation efficiency \% = 100 \times \frac{[ \frac{PSA}{Total Solids} in Nanoparticle Solution ]}{[ \frac{PSA}{Total solids} in Organic Phase ]}$$

### VI. Example 5: Chemical Modification of Poly Sialic Acid by Oxime Ligation and Thermal Hydrolysis

### 1. Oxime Ligation to PSA

### General Procedure

¹H spectra were recorded on a 600 MHz Agilent DD2. Chemical shifts calibration was using water residue peak at 4.79 ppm as reference. NMR data is represented as follows: Chemical shift, multiplicity (s=singlet, d=doublet, t=triplet, dd=doublet of doublets, m=multiplet and/or multiple resonances, br.=broad signal), J coupling, integration, and peak identity. NMR signals of 3b were assigned on the basis of ¹H NMR, ¹³C-NMR, gCOSY, gHSQC, and HMBC experiments. 0.1 M Sodium acetate buffer (pH=5) was prepared by dissolving sodium acetate in degassed MilliQ water using IM aq. HCl to adjust pH. Sialic acid oligomers were purchased from Nacalai Tesque Inc.

### General Procedure for Oxime Ligation

Synthetic procedure is illustrated in Scheme 1:

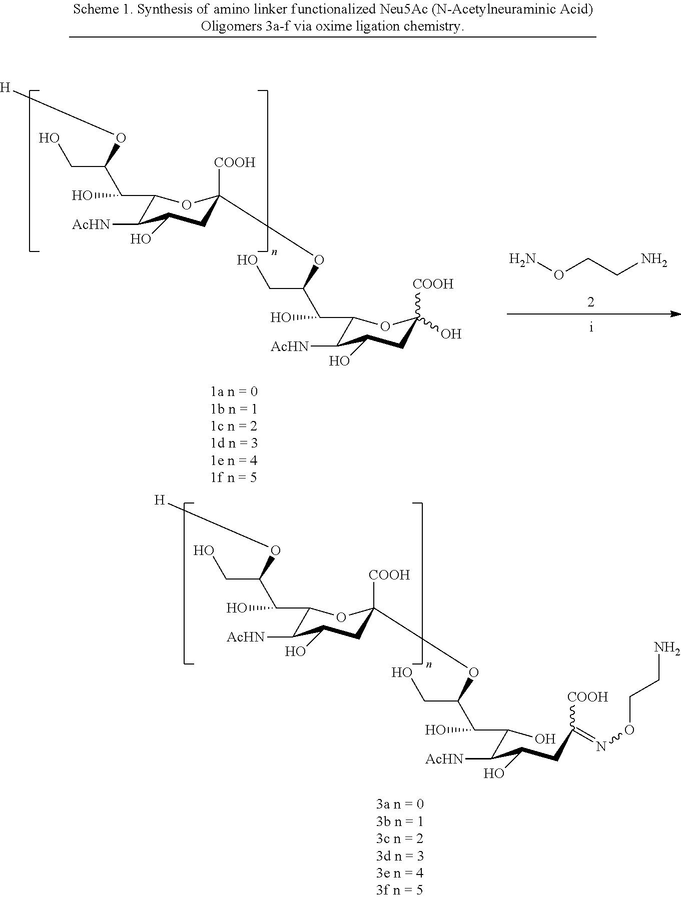

Compound 1a-f (1 eq) and compound 2 (20 eq) were dissolved in 0.2 mL NaOAc buffer (0.1 M. pH=5) and stirred at room temperature for 1 h. The reaction mixture was purified by P2 bio-gel chromatography using 0.1 M NH₄HCO₃ as the eluent. Fractions were analyzed by ESI and TLC. Pure fractions were combined, concentrated under reduced pressure at 40° C. and lyophilized to afford 3a-f as white solid.

1H-NMR and 13C-NMR spectra (where available) of the synthesized compounds are provided below:

Compound 3a (1.10 mg, 3.00 μmol, quant yield) was synthesized according to the general procedure using compound 1a (1.00 mg, 3.24 μmol) and compound 2 (4.92 mg, 64.80 μmol). ¹H NMR (600 MHz, d2o) δ 4.45 (ddd, J=9.2, 4.3, 1.3 Hz, 1H, H4), 4.43-4.34 (m, 2H, OCH₂), 3.98-3.88 (m, 2H, H5, H6), 3.88-3.81 (m, 1H, H9), 3.74 (ddd, J=10.8, 6.2, 2.7 Hz, 1H, H8), 3.67-3.60 (m, 1H, H9), 3.46 (dd, J=9.0, 1.8 Hz, 1H, H7), 3.36 (td, J=4.6, 3.0 Hz, 2H, NH₂CH₂), 2.92-2.81 (m, 1H, H3), 2.79-2.65 (m, 1H, H3), 2.08 (s, 3H CH₃Ac).

Compound 3b (1.10 mg, 1.59 μmol, quant, yield) was synthesized according to the general procedure using compound 1b (1.00 mg, 1.55 μmol) and compound 2 (2.36 mg, 31.00 μmol). ¹H NMR (600) MHz, d2o) δ 4.50-4.38 (m, 2H, H4, CH₂CH₂O), 4.27 (t, J=5.0 Hz, 1H, CH₂CH₂O), 4.04-3.76 (m, 8H), 3.74-3.60 (m, 4H), 3.60-3.52 (m, 1H), 3.41-3.26 (m, 2H, NH₂CH₂), 2.86 (dd, J=13.6, 9.5 Hz, 0.6H, H3, A), 2.78-2.65 (m, 1.60H, H3, A+B), 2.50 (d, J=7.0 Hz, 0.80H, H3, A), 2.0) (s, 3H, CH₃ Ac), 2.04 (s, 3H, CH₃ Ac), 1.79 (td, J=12.2, 2.9 Hz, 1H, H3, B). ¹³C NMR (151 MHz, d2o) δ 174.93, 174.43, 174.35 (C═O Ac), 173.56 (COOH B), 170.29 (COOH A), 169.27 (COOH A), 158.59 (C=N A), 156.72 (C=N A), 101.79, 101.76 (C2 B), 74.25, 74.20, 72.74, 72.72, 71.81, 71.79, 69.94, 69.04, 68.23, 68.02, 67.65, 67.45, 67.40, 66.26, 65.57, 62.65, 61.10, 53.59, 53.04, 51.61, 40.01 (C3 B), 38.89 (NH₂CH₂), 38.76 (NH₂CH₂), 35.49 (C3, A), 30.79 (C3 A), 21.95, 21.92, 21.88 (CH₃ Ac, A&B).

Compound 3c (0.90 mg, 0.92 μmol, 88% yield) was synthesized according to the general procedure using compound 1c (1.00 mg, 1.04 μmol) and compound 2 (1.58 mg. 20.80 μmol). ¹H NMR (600 MHz, d2o) S 4.50-4.39 (m, 2H, H4, CH₂CH₂O), 4.27 (t, J=4.9 Hz, 1H, CH₂CH₂O), 4.08 (d, J=12.1 Hz, 1H), 4.06-3.98 (m, 2H), 3.97-3.77 (m, 9H), 3.77-3.61 (m, 7H), 3.57 (d, J=9.7 Hz, 1H), 3.41-3.34 (m, 1H, NH₂CH₂), 3.31 (q, J=3.5 Hz, 1H, NH₂CH₂), 2.91-2.76 (m, 1.50H, H3), 2.74-2.62 (m, 1.50H, H3), 2.50 (d, J=7.1 Hz, 1H, H3), 2.15-2.00 (m, 9H, CH₃ Ac), 1.87-1.71 (m, 2H, H3). ¹³C NMR (151 MHz, d2o) δ 174.88, 174.40, 174.33 (C═O Ac), 172.85, 170.28 (COOH), 158.54 (C=N), 101.52, 100.79 (C2), 77.20, 74.00, 73.95, 73.33, 72.70, 71.60, 70.11, 69.02, 68.23, 68.05, 67.87, 67.80, 67.67, 67.55, 67.50, 66.11, 65.50, 62.58, 61.26, 60.69, 53.62, 53.08, 52.13, 51.63, 40.27, 39.34 (C3), 38.86, 38.77 (NH₂CH₂), 35.54, 30.85 (C3), 22.19, 21.94, 21.90 (CH₃ Ac).

Compound 3d (0.90 mg, 0.92 μmol, 59% yield) was synthesized according to the general procedure using compound 1d (2.00 mg, 1.57 μmol) and compound 2 (2.39 mg, 31.40 μmol).

¹H NMR (600 MHz, d2o) S 4.55-4.34 (m, 2H, H4, CH₂CH₂O), 4.28 (t, J=4.8 Hz, 1H, CH₂CH₂O), 4.21-3.54 (m, 28H), 3.43-3.27 (m, 2H, NH₂CH₂), 2.95-2.59 (m, 4H, H3), 2.51 (d, J=6.8 Hz, 1H, H3), 2.19-2.00 (m, 12H, CH₃ Ac), 1.88-1.69 (m, 3H, H3).

Compound 3e (0.90 mg, 0.56 μmol, 89% yield) was synthesized according to the general procedure using compound 1e (1.00 mg, 0.63 μmol) and compound 2 (0.96 mg, 12.60 μmol). ¹H NMR (600 MHz, d2o) δ 4.52-4.36 (m, 2H, H4, CH₂CH₂O), 4.27 (dd, J=6.7, 3.2 Hz, 1H, CH₂CH₂O), 4.19-3.53 (m, 37H), 3.43-3.27 (m, 2H, NH₂CH₂), 2.90-2.59 (m, 5H, H3), 2.49 (d, J=6.8 Hz, 1H, H3), 2.15-1.97 (m, 15H, CH₃ Ac), 1.76 (q, J=11.7 Hz, 4H, H3).

Compound 3f (3.60 mg, 1.89 μmol, 90% yield) was synthesized according to the general procedure using compound 1f (4.00 mg, 2.11 μmol) and compound 2 (3.21 mg, 42.20 μmol). ¹H NMR (600 MHz, d2o) δ 4.53-4.37 (m, 2H, H4, CH₂CH₂O), 4.28 (t, 1H, CH₂CH₂O), 4.20-3.54 (m, 41H), 3.44-3.27 (m, 2H, NH₂CH₂), 2.93-2.61 (m, 6H, H3), 2.50 (d, J=6.9 Hz, 1H, H3), 2.23-1.95 (m, 18H, CH₃ Ac), 1.90-1.65 (m, 5H, H3).

### 2. Thermal Hydrolysis of PSA and its Purification

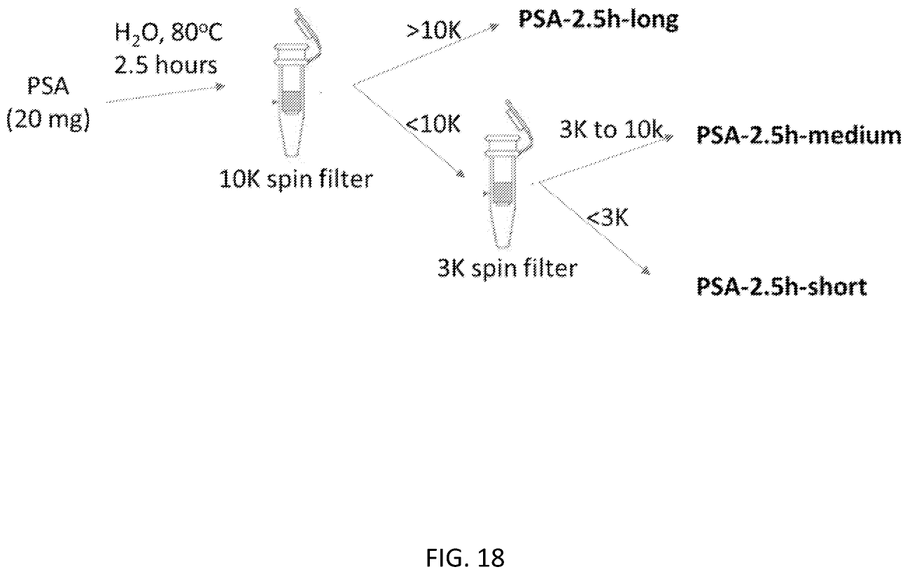

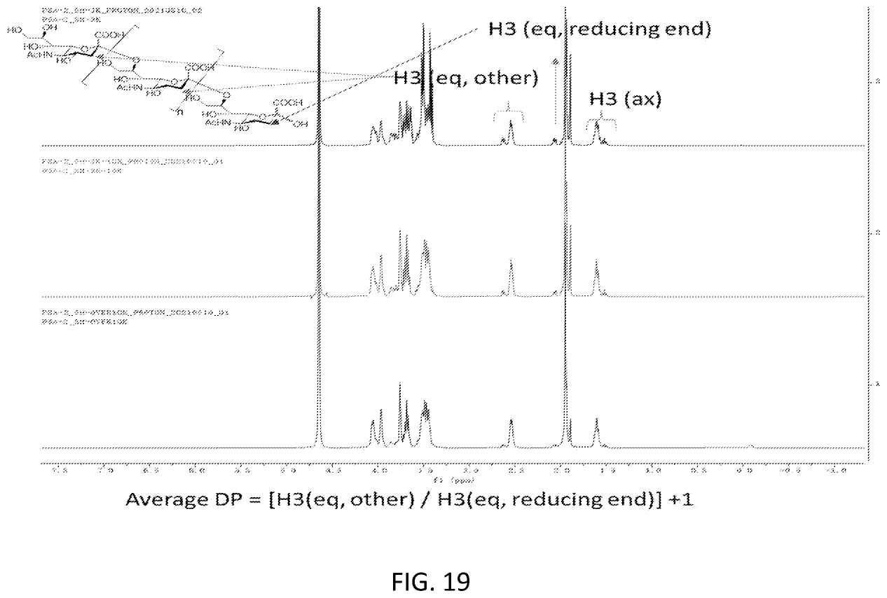

Colominic acid (20 mg) was dissolved in H₂O (2 mL) and the solution was stirred at 80° C. for 2.5 h after which it was purified by ultrafiltration using 10 K spin filter (Nanosep centrifugal devices, 4 times). The upper residue was collected and labeled as “PSA-2.5 h-long”. (FIG. 18.) The filtrate of the 10 K spin filter was concentrated under reduced pressure to remove most of the solvent and further purified by ultrafiltration using 3 K spin filter (Nanosep centrifugal devices, 4 times). The upper residue was collected and labeled as “PSA-2.5 h-medium”. Similarly, the filtration of 3 K spin filter was concentrated under reduced pressure to remove most of the solvent, and labeled “PSA-2.5 h-short”. The three fractions were lyophilized, and analyzed by ¹H-NMR in D20 to determine the Degree of Polymerization (DP) (FIG. 19).

PSA-2.5 h-long: 8.7 mg, 43.5% yield (from colominic acid), average DP=20.1, average Mw=5884 (calculated by DP value); PSA-2.5 h-medium: 3.7 mg, 18.5% yield (from colominic acid), average DP=12.8, average Mw=3757 (calculated by DP value): PSA-2.5 h-short: 7.6 mg, 38.0% yield (from colominic acid), average DP=6.8, average Mw=2011 (calculated by DP value).

3. Modification of PSA with a Linker

With reference to FIG. 18, PSA-2.5 h-long/medium/short (1 eq), compound 2 (20 eq) in 0.2 mL NaOAc buffer (0.1 M, pH=5) was stirred at room temperature for 2 hours. The reaction mixture was purified by P2/P4 bio-gel chromatography using 0.1 M NH₄HCO₃ as eluent. Fractions were analyzed by ESI and TLC. Fractions that contain product were combined, concentrated under reduced pressure at 40° C. and lyophilized to afford PSA-2.5 h-long/medium/short-linker as white solid.

¹H-NMR study of the compounds before and after linker ligation were conducted. By assigning the chemical shift of key proton signals, the successful introduction of linker was confirmed and the DP calculated by the ratio of the integration of the linker “CH₂” and Acetyl “CH₃” of the sialic acid.

The following linker-conjugated PSA compounds were obtained: PSA-2.5 h-long (5 mg) was used as starting material and the product was purified by P4 bio-gel chromatography to yield PSA-2.5 h-long-linker. (0.9 mg, 18.0% yield, average DP=16.61, average Mw=4910); PSA-2.5 h-medium (3.7 mg) was used as starting material and the product was purified by P4 bio-gel chromatography to yield PSA-2.5 h-medium-linker. (1.6 mg, 43.2% yield, average DP=13.33, average Mw=3955); 2.0 mg PSA-2.5 h-short was used as starting material and the product was purified by P2 bio-gel chromatography to yield PSA-2.5 h-short-linker. (1.3 mg, 65% yield, average DP=5.17, average Mw=1580)

### Biological Experiments and Methods

Batches of microparticles are referred to herein based on identification found in table 4.

### I. Example 6: Affinity of Exemplary Nanoparticles to Selected Targets

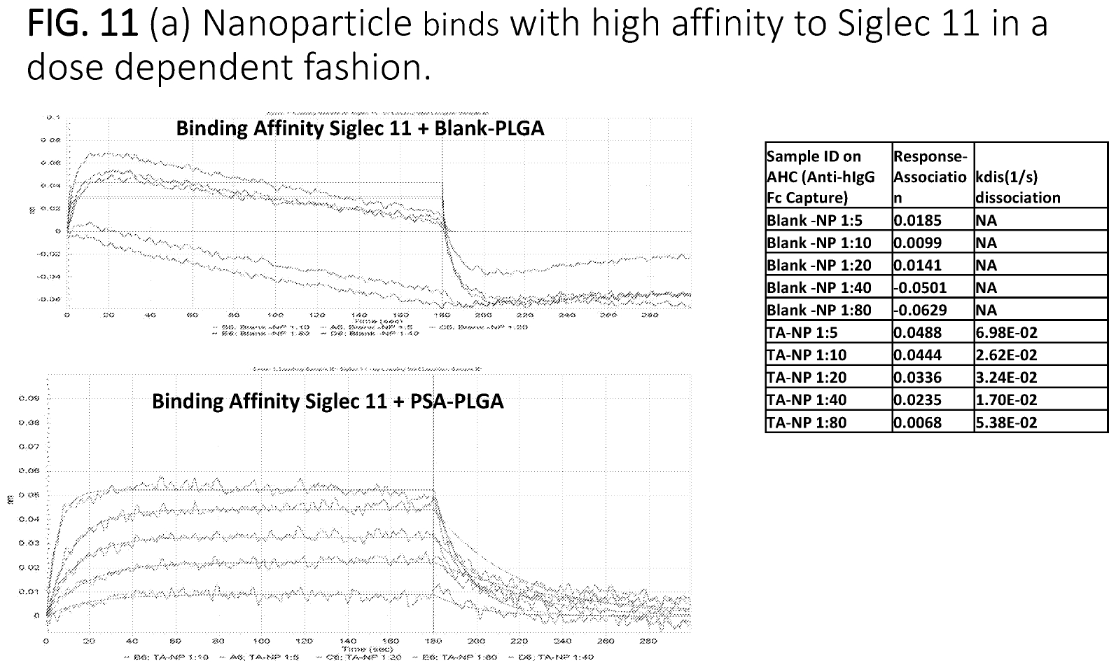

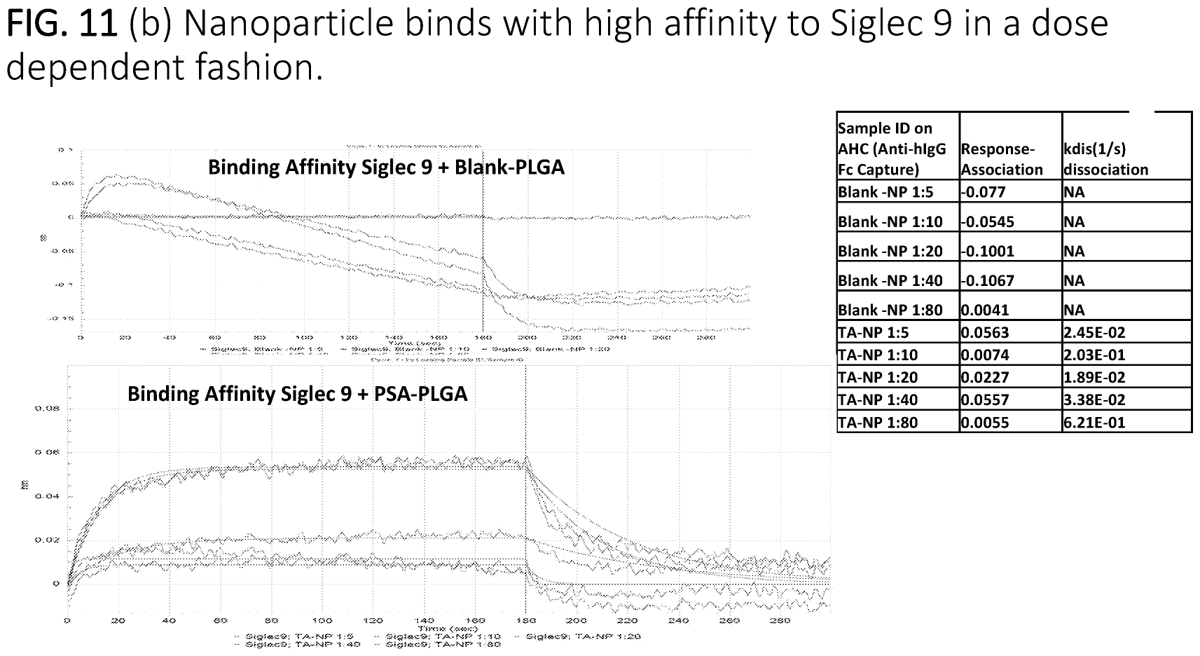

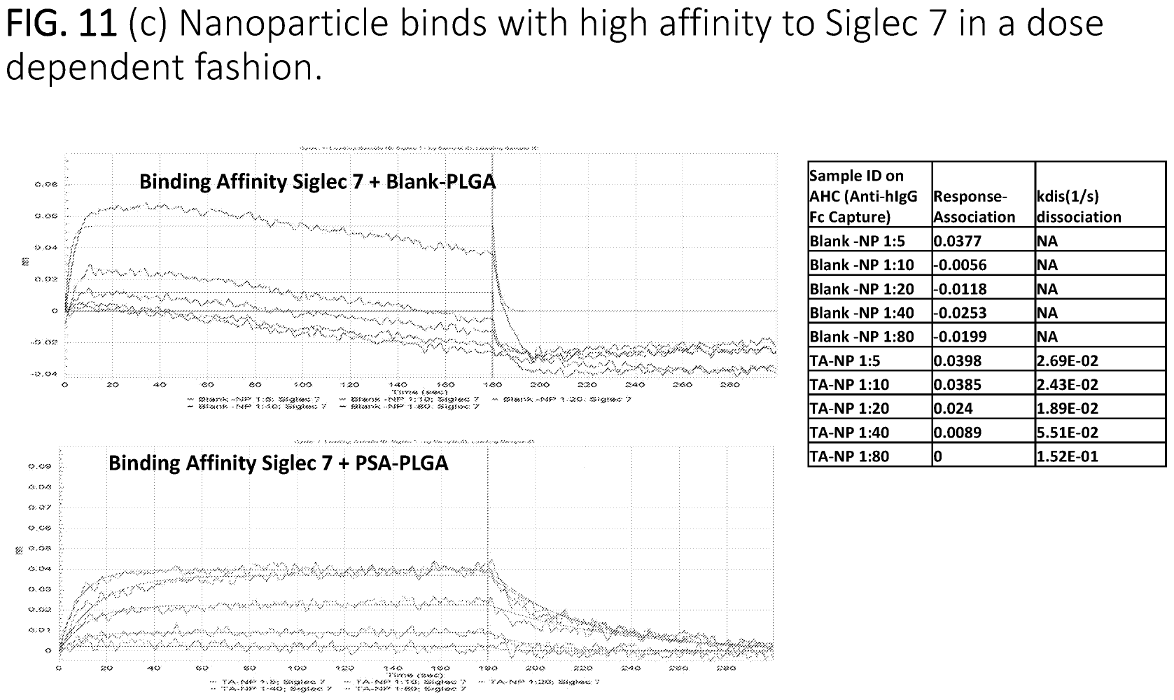

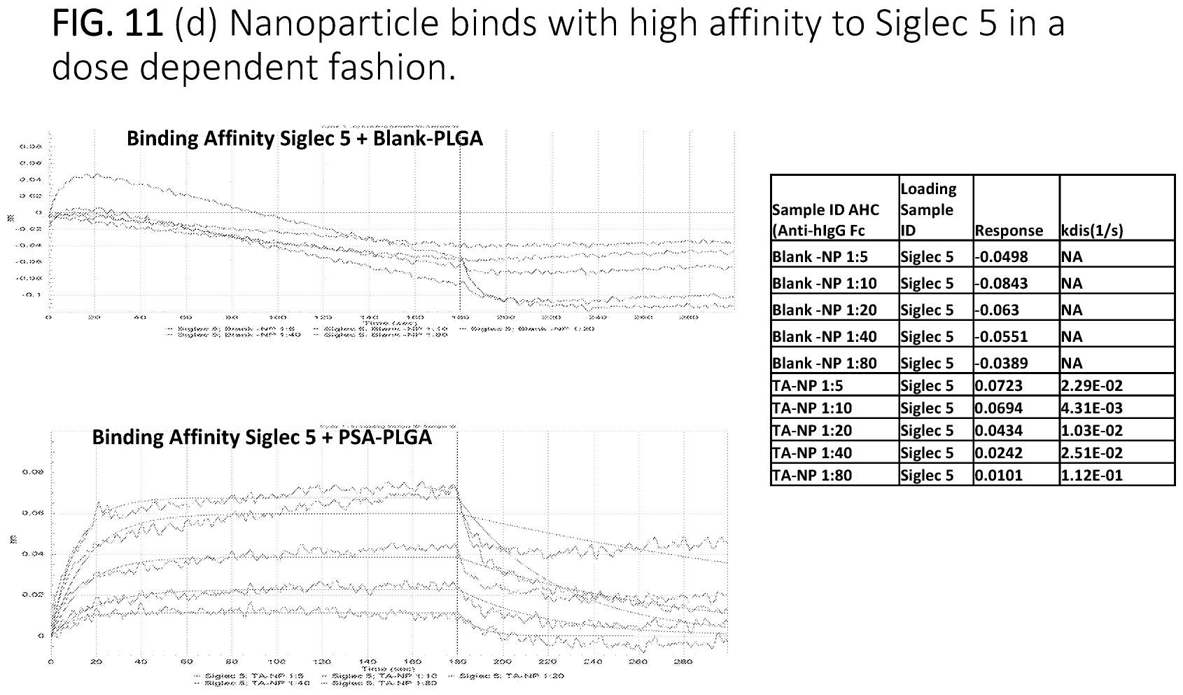

Octet assay uses the technology called Bio-Layer Interferometry (BLI). Bio-Layer Interferometry is a label-free technology for measuring biomolecular interactions. It is an optical analytical technique that analyzes the interference pattern of white light reflected from two surfaces, a layer of immobilized protein on the biosensor tip, and an internal reference layer (FIGS. 11A, 11B, and 11C). Any change in the number of molecules bound to the biosensor tip causes a shift in the interference pattern that can be measured in real-time. Only molecules binding to or dissociating from the biosensor can shift the interference pattern and generate a response profile on the Octet® System. Unbound molecules, changes in the refractive index of the surrounding medium, or changes in flow rate do not affect the interference pattern. This is a unique characteristic of BLI and extends its capability to perform in crude samples used in applications for protein, protein binding, quantitation, affinity, and kinetics.

Affinity of exemplary nanoparticles and different Siglec receptors were measured using an Octet assay as follows: Probe sensors were blocked with PBS+1% BSA. Different Siglec Fc fusion proteins specific for Siglecs 11, 9, 7, and 5 at a concentration of 25 μg/mL in PBS were captured using anti-human Fc capture antibody coated (AHC) dip and read biosensors. An association curve was generated when the probe was dipped into wells containing analytes at different dilutions. The analytes were the exemplary nanoparticles or nanoparticles without ligands that have been diluted at ratios of 1:10, 1:20, 1:40, 1:80, 1:160 in PBS. The dissociation rate (off rate) was measured when the probe is dipped into PBS. The binding response between different concentrations of the exemplary nanoparticles in assay buffer (1×PBS) with anti-human Fc fusion protein and Siglec 11, 9, 7, and 5 at a concentration of 25 μg/mL were measured. The graphs display the binding response by a nanometer shift which was directly proportional to the amount bound in the BLI platform vs. the time (seconds). For reference, a sensor loaded with buffer alone was used. FIG. 11a shows the binding affinity between the exemplary nanoparticles and Siglec 11, FIG. 11b shows the binding affinity between the exemplary nanoparticles and Siglec 9, FIG. 11c shows the binding affinity between the exemplary nanoparticles and Siglec 7, and FIG. 11d shows the binding affinity between the exemplary nanoparticles and Siglec 5. The binding affinities are also depicted in the table below:

| | Loading | | |
|---|---|---|---|
| Sample ID | Sample ID | Response | kdis(1/s) |
| Blank -NP 1:5 | Siglec 11 | 0.0185 | NA |
| Blank -NP 1:10 | Siglec 11 | 0.0099 | NA |
| Blank -NP 1:20 | Siglec 11 | 0.0141 | NA |
| Blank -NP 1:40 | Siglec 11 | −0.0501 | NA |
| Blank -NP 1:80 | Siglec 11 | −0.0629 | NA |
| PSA-PLGA 1:5 | Siglec 11 | 0.0488 | 6.98E−02 |
| PSA-PLGA 1:10 | Siglec 11 | 0.0444 | 2.62E−02 |
| PSA-PLGA 1:20 | Siglec 11 | 0.0336 | 3.24E−02 |
| PSA-PLGA 1:40 | Siglec 11 | 0.0235 | 1.70E−02 |
| PSA-PLGA 1:80 | Siglec 11 | 0.0068 | 5.38E−02 |
| Blank -NP 1:5 | Siglec 7 | 0.0377 | NA |
| Blank -NP 1:10 | Siglec 7 | −0.0056 | NA |
| Blank -NP 1:20 | Siglec 7 | −0.0118 | NA |
| Blank -NP 1:40 | Siglec 7 | −0.0253 | NA |
| Blank -NP 1:80 | Siglec 7 | −0.0199 | NA |
| PSA-PLGA 1:5 | Siglec 7 | 0.0398 | 2.69E−02 |
| PSA-PLGA 1:10 | Siglec 7 | 0.0385 | 2.43E−02 |
| PSA-PLGA 1:20 | Siglec 7 | 0.024 | 1.89E−02 |
| PSA-PLGA 1:40 | Siglec 7 | 0.0089 | 5.51E−02 |
| PSA-PLGA 1:80 | Siglec 7 | 0 | 1.52E−01 |
| Blank -NP 1:5 | Siglec 5 | −0.0498 | NA |
| Blank -NP 1:10 | Siglec 5 | −0.0843 | NA |
| Blank -NP 1:20 | Siglec S | −0.063 | NA |
| Blank -NP 1:40 | Siglec 5 | −0.0551 | NA |
| Blank -NP 1:80 | Siglec 5 | −0.0389 | NA |
| PSA-PLGA 1:5 | Siglec 5 | 0.0723 | 2.29E−02 |
| PSA-PLGA 1:10 | Siglec 5 | 0.0694 | 4.31E−03 |
| PSA-PLGA 1:20 | Siglec 5 | 0.0434 | 1.03E−02 |
| PSA-PLGA 1:40 | Siglec 5 | 0.0242 | 2.51E−02 |
| PSA-PLGA 1:80 | Siglec 5 | 0.0101 | 1.12E−01 |
| Blank -NP 1:5 | Siglec9 | −0.077 | NA |
| Blank -NP 1:10 | Siglec9 | −0.0545 | NA |
| Blank -NP 1:20 | Siglec9 | −0.1001 | NA |
| Blank -NP 1:40 | Siglec9 | −0.1067 | NA |
| Blank -NP 1:80 | Siglec9 | 0.0041 | NA |
| PSA-PLGA 1:5 | Siglec9 | 0.0563 | 2.45E−02 |
| PSA-PLGA 1:10 | Siglec9 | 0.0074 | 2.03E−01 |
| PSA-PLGA 1:20 | Siglec9 | 0.0227 | 1.89E−02 |
| PSA-PLGA 1:40 | Siglec9 | 0.0557 | 3.38E−02 |
| PSA-PLGA 1:80 | Siglec9 | 0.0055 | 6.21E−01 |

### II. Example 7: Non-Toxicity Profile of Exemplary Nanoparticles with Respect to Peripheral Blood Monocytes (PBMCs)

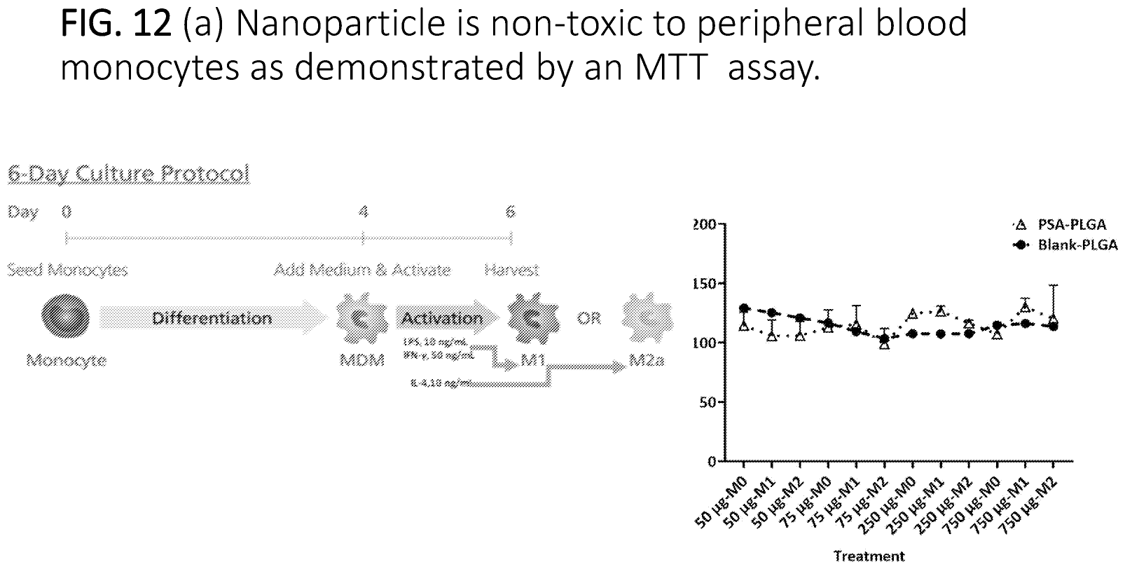

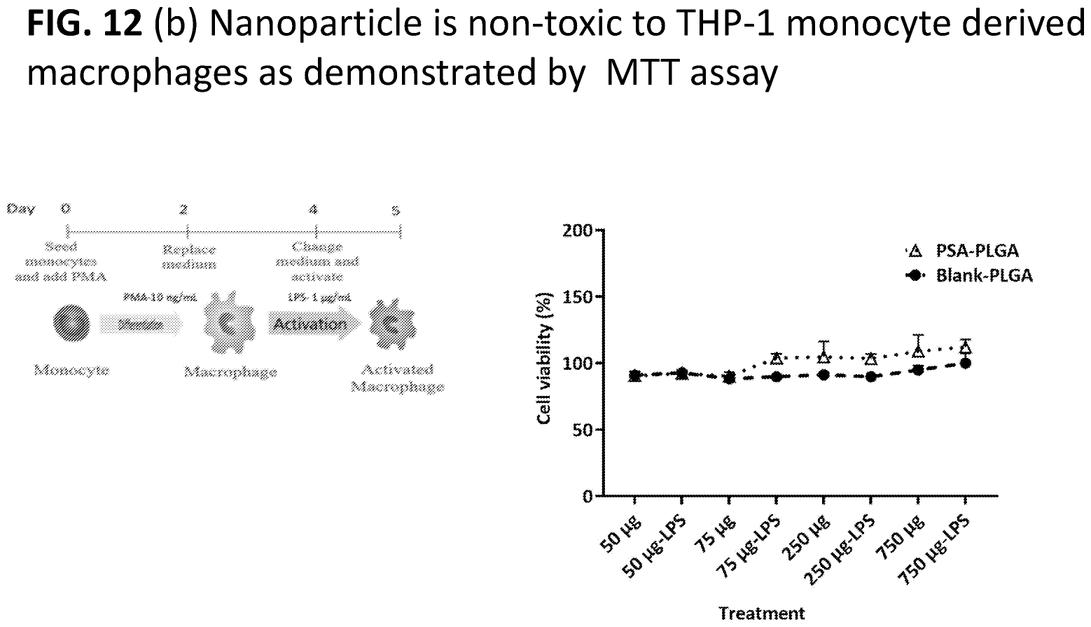

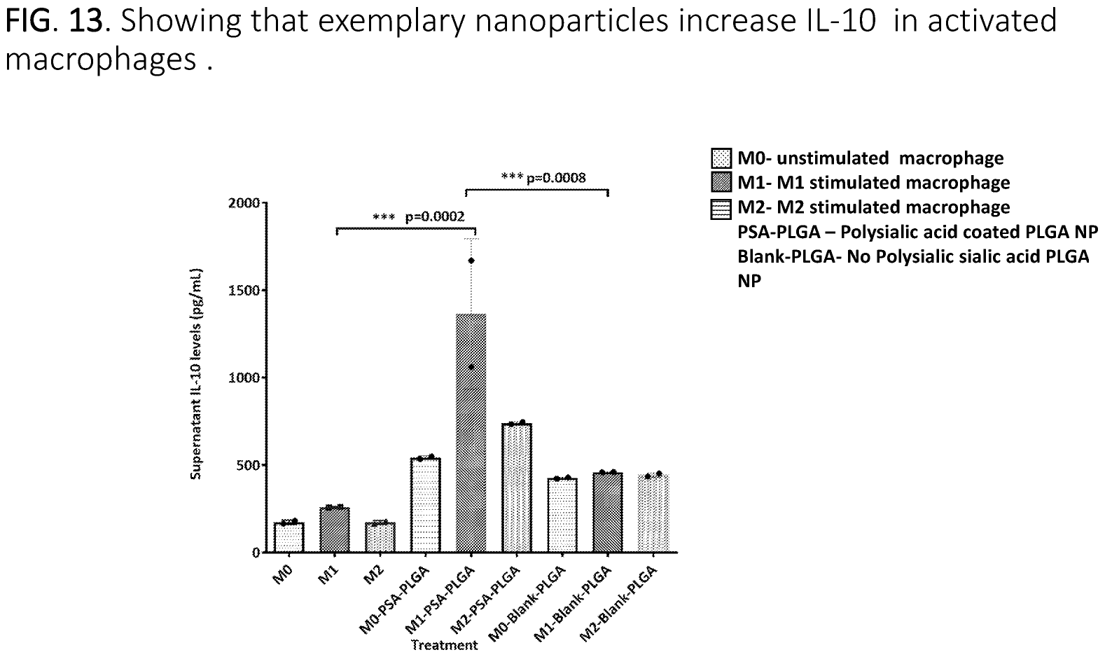

Analysis of percentage of cell survival by MTT assay on PBMC-derived macrophages was conducted in order to assess the non-toxicity profile of the exemplary nanoparticles. The cytotoxicity of exemplary nanoparticles labeled with FITC was evaluated on PBMC cells. The PBMCs were obtained from healthy donors and were activated to M1 phenotype (using Hu IFN-y 50 ng/ml, LPS 10 ng/ml) and M2 phenotype (using hu IL-4-10 ng/ml) for 48 hours. The cells were then treated with 50-750 μg/mL of the exemplary nanoparticles for 24 hours. MTT [3-(4,5-dimethylthiazol-2-yl)-2,5-diphenyltetrazolium bromide assay was used to determine cell survival. Macrophages cells (M0, M1, M2) were seeded in a 96 well plates at a density of 100×10cells/200 μl media in each well. The cells were incubated for 24 h with 50-750 μg/mL of the exemplary nanoparticles and control nanoparticles lacking a ligand. After 24 hours, cells were washed and incubated with 1 mg/mL 1 of MT for 3-4 h at 37° C. The resulting formazan crystals were dissolved in 100 μl MTT solubilization buffer and the absorbances were measured at 570 nm using a Spectra iMax-plate reader, the values were compared to the control cells. Graphs represents percent cell viability when compared to control untreated cells. FIG. 12(a) reflects that the exemplary nanoparticles are non-toxic to peripheral blood monocytes as demonstrated by MTT assay.

### III. Example 8: Non-Toxicity Profile of Exemplary Nanoparticles with Respect to Macrophages

In order to determine the non-toxicity profile of exemplary nanoparticles, an in-vitro cytotoxicity assay on THP-1 monocytes derived macrophages was conducted. THP-1 cells were seeded in a 96 well plates at a density of 100×10³ cells/200 ul in each well. THP-1 monocytes differentiated macrophages were treated with 50-750 μg/mL of the exemplary nanoparticles for 24 hours. Cells were differentiated using 10 ng/mL phorbol-12-myristate-13-acetate (PMA) and activated using lipopolysaccharide (LPS) at 1 μg/mL. The cells were incubated for 24 hours with 50-750 μg/mL dose of exemplary nanoparticles and control nanoparticles lacking a ligand. After 24 hours, cells were washed and incubated with 1 mg/mL of MTT for 3-4 hi at 37° C. The resulting formazan crystals were dissolved in 100 μl of MTT solubilization buffer and the absorbances were measured at 570 nm using a Spectra iMax plate reader, the values were compared to the control cells. Graphs represents percent cell viability when compared to control untreated cells. FIG. 12(b) demonstrates that the exemplary nanoparticles are non-toxic to THP-1 derived macrophages.

### IV. Example 9: Effect of Exemplary Nanoparticles on IL-10 in Activated Macrophages

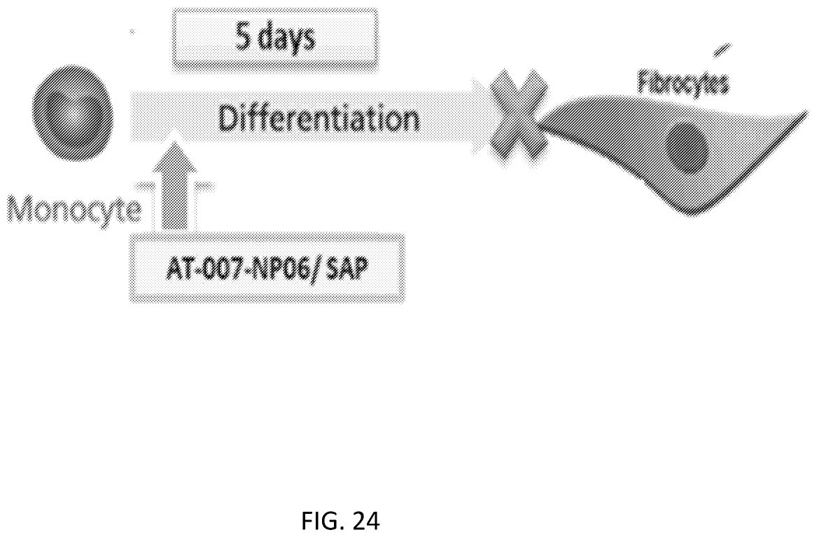

The ability of exemplary nanoparticles to upregulate IL-10 production in human PBMC-derived macrophages was demonstrated as follows. PBMCs from healthy donors were activated to M1 phenotype (using Hu IFN-y 50 ng/mL, LPS 10 ng/mL) and M2 phenotype (using hu IL-4-10 ng/ml) for 48 hours, and then treated with 75 μg/mL of exemplary nanoparticles and control nanoparticles for 24 hours. The supernatant from the wells were collected and assayed for IL-10 by ELISA (R&D systems) Graphs represent IL-10 protein levels showing upregulation of IL-10 24 post treatment with the exemplary nanoparticles when compared to control nanoparticles containing no ligand and the no treatment control groups. Sidak's multiple comparisons test M1 vs. M1-PSA-PLGA ** p=0.0002, M1-PSA-PLGA vs. M1-Blank-PLGA *** p=0.0008 These results are shown in FIG. 24.

### V. Example 10: Effect of Exemplary Nanoparticles on CFH Production

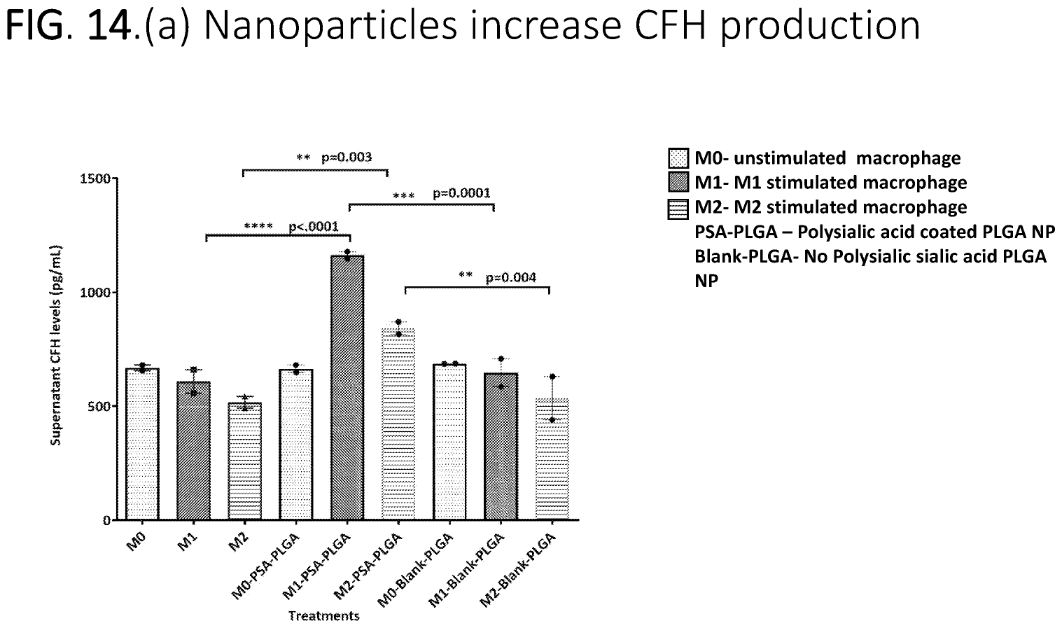

The ability of exemplary nanoparticles to upregulate Complement factor H (CFH) production in human PBMC-derived macrophages was demonstrated as follows. PBMCs from healthy donors were activated to M1 phenotype (Hu IFN-y 50 ng/mL. LPS 10 ng/mL) and M2 phenotype (hu IL-4-10 ng/mL) for 48 hours, and then treated with 75 μg/mL of exemplary nanoparticles and control nanoparticles lacking a ligand for 24 hours. The supernatant collected from the wells were assayed for CFH by ELISA (Abcam). The graphs in FIG. 14(a) represent CFH protein levels showing upregulation of CFH 24-post treatment with the exemplary nanoparticles when compared control nanoparticles lacking a ligand and the no treatment groups. Sidak's multiple comparisons test M1 vs. M1-PSA-PLGA **** p<0.0001, M2 vs M2-PSA-PLGA ** p=0.0032 M1-PSA-PLGA vs. M1-Blank-PLGA ***p=0.0001 M2-PSA-PLGA vs. M2-Blank-PLGA ** p:=0.004

### VI. Example 11: The Ability of Exemplary Nanoparticles to Decrease Complement C3 Levels in Human PBMC-Derived Macrophages

PBMCs from healthy donors were activated to the M1 phenotype (with Hu IFN-y 50 ng/mL, LPS 10 ng/mL) and the M2 phenotype (with hu IL-4-10 ng/ml) for 48 hours, and then treated with 75 μg/mL of the exemplary nanoparticles or control nanoparticles lacking a ligand for 24 hours. Supernatants collected were assayed for C3 levels by ELISA (Abcam). FIG. 14(b) shows the results and reflects Complement C3 protein levels showing clear suppression of Complement C3 levels 24 hours after treatment of the exemplary nanoparticles on M0 and M1 macrophages as compared to the control nanoparticles lacking a ligand and the no treatment groups. Sidak multiple comparisons test M1-PSA-PLGA vs M1-Blank-PLGA ** p=0.007.

### VII. Example 12: Complement Regulation Mediated by AT-007-NP04 Nanoparticle

The complement pathway is a vital component of the innate immune system that results in non-specific cellular lysis of cell membranes via an effector membrane attack complex. The cascade can proceed via 3 separate pathways the classical, alternative and lectin pathway. The classical pathway is triggered by antibodies or antibody complexes. The alternative pathway is constitutively activated and is amplified when a non host cell or molecular pattern is encountered. The lectin pathway is activated when a non self associated sugar residue is encountered such as what is seen in the cell walls of pathogens.

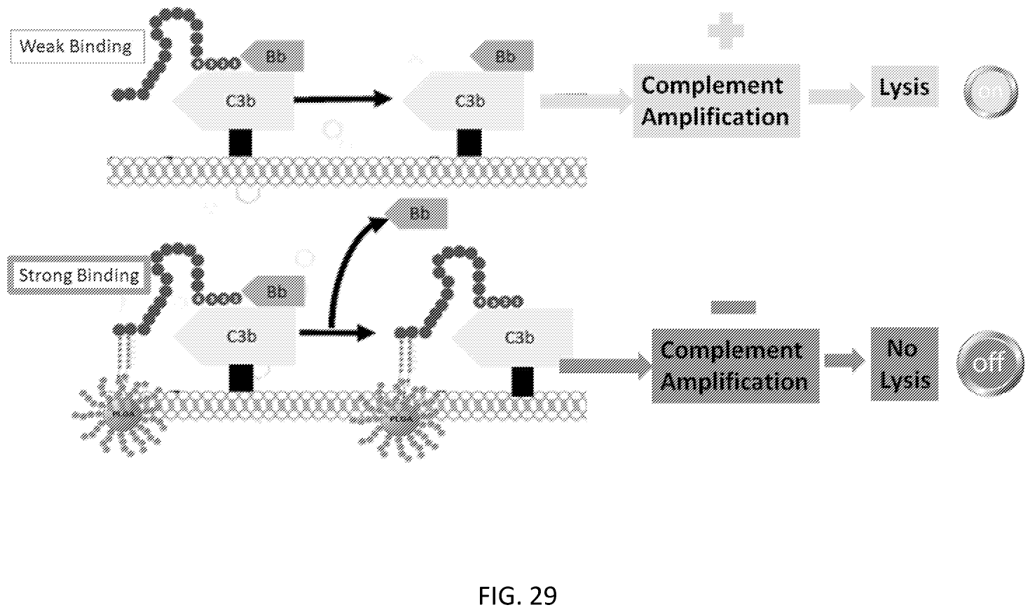

These pathways share a rate limiting step which is the formation of C3 convertase (C3bBb). Because of the potential for off target cellular lysis, the formation of C3 convertase is tightly regulated by Complement factor H (CFH). CFH is in turn regulated by its ability to bind self associated molecular patterns (SAMP, sialic acids) which allow CFH to bind C3b and reduce its ability to bind Bb. When CFH is not bound to a SAMP C3b binds Bb with high affinity and results in the propagation and amplification membrane attack complex formation and cellular lysis of host and pathogen cells alike (FIG. 28). The nature of our invention is to present sialic acid ligands to mimic self associated molecular patterns. Below we demonstrate how our AT-007-NP04 nanoparticle can bind and activate CFH which in turn binds C3b with higher affinity and reduces formation of C3b (FIG. 29).

### 1. Ability of AT-007-NP04 Enhance Binding of CFH to C3B

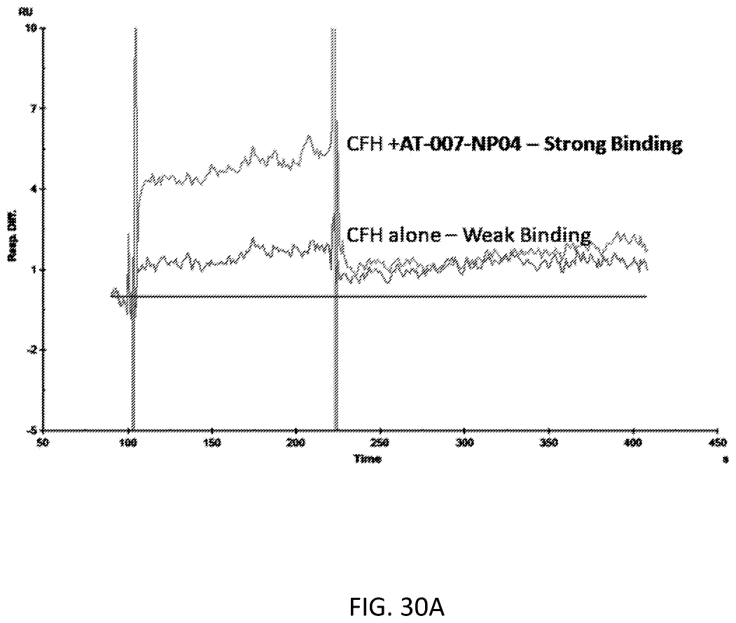

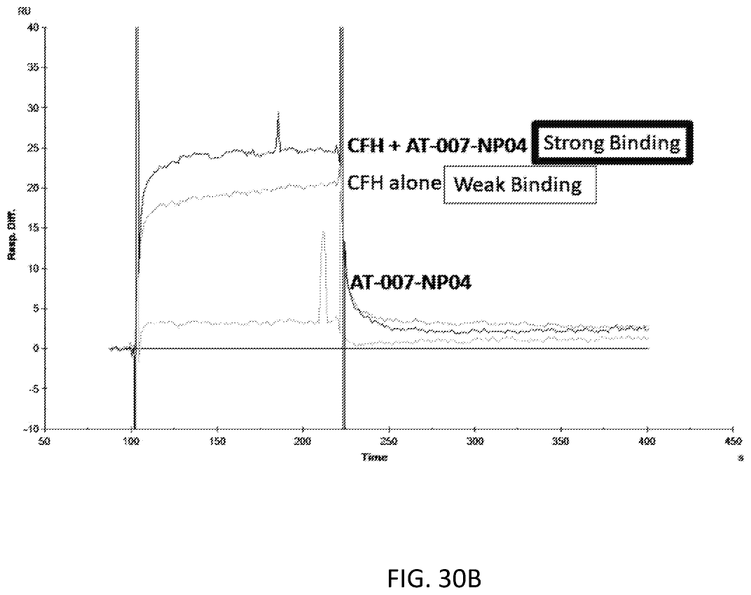

To determine if the AT-007-NP04 nanoparticle could bind CFH in order to enhance CFH binding to C3b, a Biacore assay was performed. In this example, C3b was immobilized to Fc2 at 1600 RU Fc3 at 1450 RU, 950 RU. The assay was performed using, buffer alone, human complement factor H at 200 nm (FIG. 30A) and 100 nM (FIG. 30B) and human complement factor H+AT-007-NP04 at 1:20 dilution (incubation at room temperature for 10 minutes). When AT-007-NP04 was incubated with CFH, there was increase binding of CFH to C3B. The binding of C3B by CFH occurs when CFH is conformationally changed. In vivo, CFH must bind a self associated molecular pattern presented by host cells to conformationally change CFH and enhance C3B binding. This example demonstrates the ability of AT-007-NP04 to behave as a self associated molecular pattern in the absence of a host cells. This provides direct evidence that AT-007-NP04 can down regulate the complement cascade by activating CFH. Without AT-007-NP04, CFH does not bind C3B since it is not activated.

The setup of the first experiment is provided in the following table:

| Ligand | Analyte | Conc |
|---|---|---|
| C3b (1600 RU) | Factor H alone | 200 nM |
| | Factor H + AVDNP | |

In this experiment, C3b was immobilized on to Biacore plates and complement factor H with or without AT-007-NP04 was flown through into solution and the change in refractive index was measured in real time. The graph in FIG. 30A is plotted as response of resonance units (RUs) versus time (a sensorgram).

The setup of the second experiment is shown in the following table:

| Ligand | Analyte | Concentration |
|---|---|---|
| C3b (950 RU) | Complement Factor H | 100 nM |
| C3b (950 RU) | AT-07-NP04 | 130 μg/ml |
| C3b (950 RU) | Complement Factor H + | 100 nM + 130 μg/ml |
| | AT-007-NP04 | |

In this experiment, C3b was immobilized on to Biacore plates and complement factor H with or without AT-007-NP04 was flown through into solution and the change in refractive index was measured in real time. The graph in FIG. 30B is plotted as response of resonance units (RUs) versus time (a sensorgram).

### 2. Ability of AT-007-NP06 Nanoparticle to Directly Prevent Complement Activation Via Alternative or Classical Pathway

In order to determine if the AT-007-NP06 nanoparticle can inhibit the alternative and classical pathway a complement activation assay using defibrinated serum incubated with IgM (classical complement activator), zymogen and LPS (both alternative complement activators) with and without AT-007-NP06 nanoparticle. To perform this assay, plates were blocked with 1% BSA prior to incubation with defibrinated serum, zymogen, LPS, or IgM, +/−AT-007-NP06 nanoparticle or sucrose. After 3 hour incubation, anti-C3b antibody is added followed 1 hour later with Strep HRP and then read 20 minutes later by a plate reader.

The results of this assay demonstrate that the addition of the zymogen and LPS demonstrated a statistically significant decline in C3b formation when incubated with AT-007-NP06 nanoparticle and zymogen and IgM, LPS treated serum showed a trend but was not statistically significant mainly because the LPS sucrose arm was unable to significantly activate complement and C3b upregulation.

This particular example shows that AT-007-NP06 nanoparticle is able to inhibit complement amplification for both the classical and alternative pathway via the prevention of the formation C3b. C3b formation is the direct binding target of CFH.

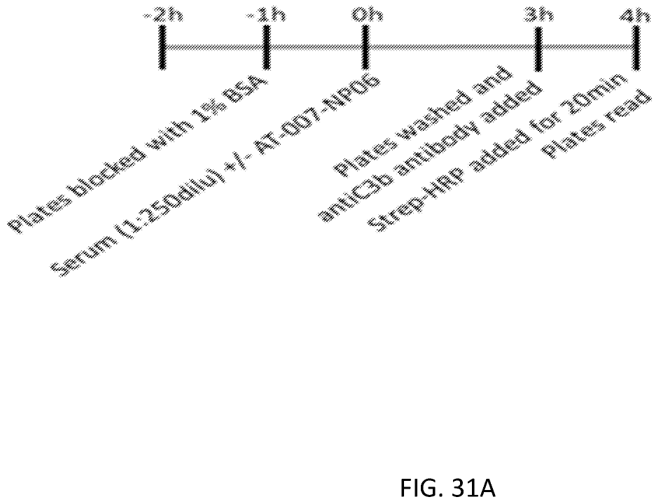

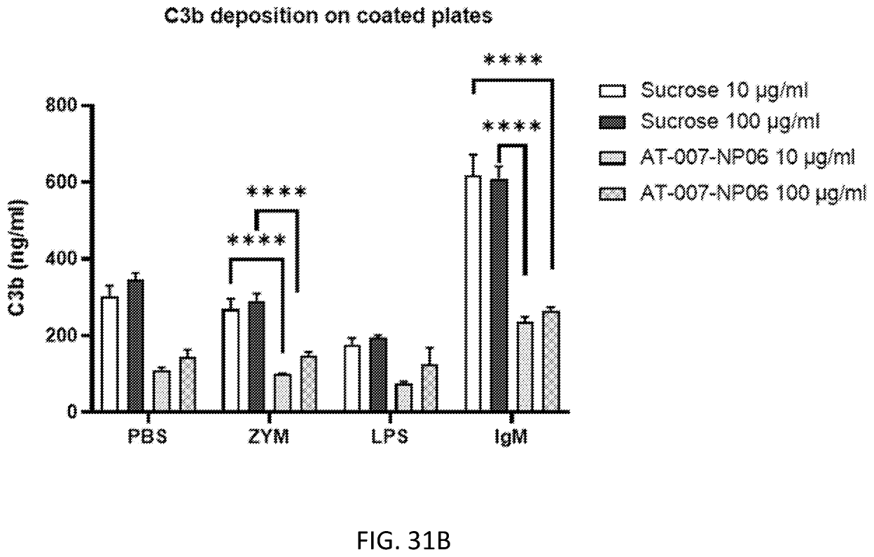

The setup of the experiment is shown in FIG. 31A (Defibrinated blood serum for C3b deposition on coated plates), and the results are shown in FIG. 31B.

From the previous example AT-007-NP06 nanoparticle with CFH increased binding of CFH to C3b. The combination of these 2 examples demonstrates the ability of AT-007-NP06 nanoparticle to increase binding of CFH to C3b which results in the reduction in c3b triggered by either alternative or classical complement pathways.

### 3. Demonstration of Direct Binding of AT-007-NP04 Nanoparticle to CFH.

In order to determine if the AT-007-NP04 nanoparticle can bind to CFH directly we precoated plates with His and incubated with Human CFH his tag protein which fixed CFH to the plate. The plates were then incubated with AT-007-NP04 nanoparticles that contained a polyethylene glycol (PEG) linker in differing concentrations in order to determine if we could calculate an IC50 and a dose kinetic binding relationship. The plates were then incubated with anti-PEG biotin and read on and ELISA plate reader at 490 nm absorbance.

This assay demonstrated and S binding curve with and IC50 of approximately 2 mg/ml. These results confirm the ability of the AT-007-NP04 nanoparticle invention to bind human CFH in a dose dependent fashion providing it potential pharmaceutical and therapeutic properties.

### EJISA Based Binding Affinity of AT-007-NP04 and AT-0097-NP06 to CFH Protein.

His tag human CFH protein (1 ug/ml concentration) (Antibody Clone-Catalog No-ABIN1079269) was pre coated on Nickel Coated Plates ELISA (Thermo Fisher Catalog No 15142 Pierce Nickel Coated Clear Plates) overnight at RT. These are ideal for analysing polyhistidine-tagged fusion proteins (His tag) by ELISA based methods. Proteins that contain a succession of several histidine residues at the amino or carboxyl terminus have a strong binding affinity for metal. Proteins containing polyhistidine-tagged fusion proteins can be added directly to the plates.

Following the standard manufacturer's ELISA protocol and reagents from R&D Systems, His tag-CFH plates were washed and AT-007-NP04 (concentration ranging from 10 mg/ml-0.001 mg/ml) and AT-004-NP06 (0.1 mg/ml-0.001 mg/ml) of total solids of Nanoparticles (NP) were added for 3 hrs at room temperature. The PEG in the nanoparticle was detected using anti PEG Biotin (Recombinant Biotin Anti-Polyethylene glycol antibody-Abcam ab53449) incubated for 1 hr at room temperature. Streptavidin HRP (1:40) was used to bind to the biotin and developed using color reagents (R&D Systems) Absorbance was measured at 490 nm. Graphs suggests AT-007-NP04 exhibiting higher binding affinity towards CFH protein when compared to blank nanoparticle controls

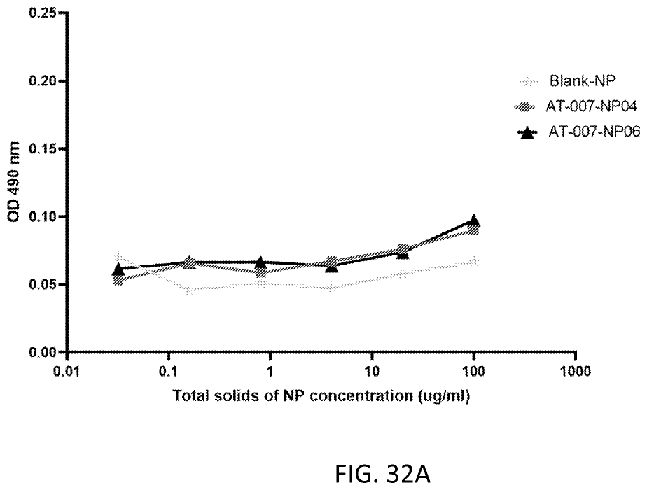

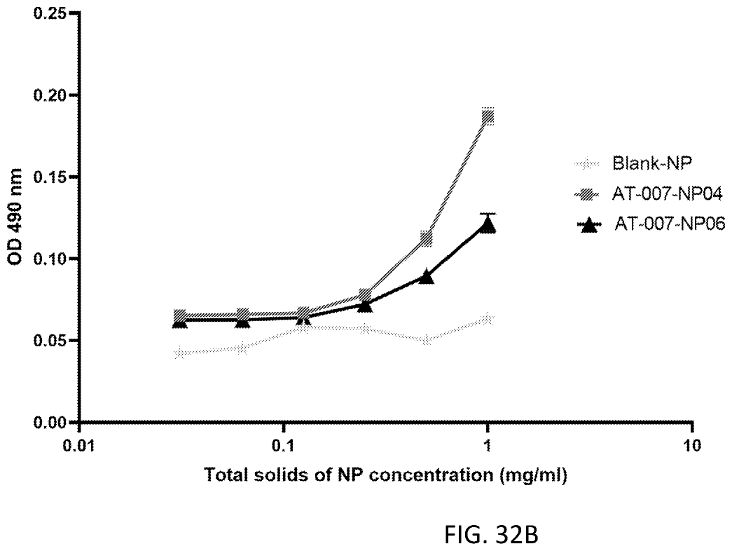

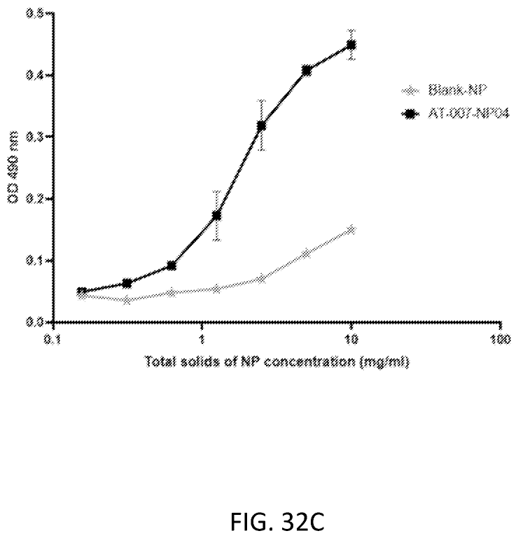

The results of this experiment are presented in FIG. 32A, 32B, and 32C. As can be seen, Maximum concentration of 100 ug/ml (A) of AT-007-NP06 and AT-007-NP04 exhibited no difference in the binding affinity towards CFH protein when compared to blank nanoparticles. Maximum concentration of 1 mg/ml (B) exhibited significant difference in the binding affinity towards CFH protein w % ben compared to blank nanoparticles at high concentration. Maximum concentration of 10 mg/ml (C) of AT-007-NP06 exhibited sigmodial S binding curve with a significant difference in the binding affinity towards CFH protein when compared to blank nanoparticles.

### VIII. Example 13: Effect of Exemplary Nanoparticles on TNF-Alpha in LPS-Activated Macrophages

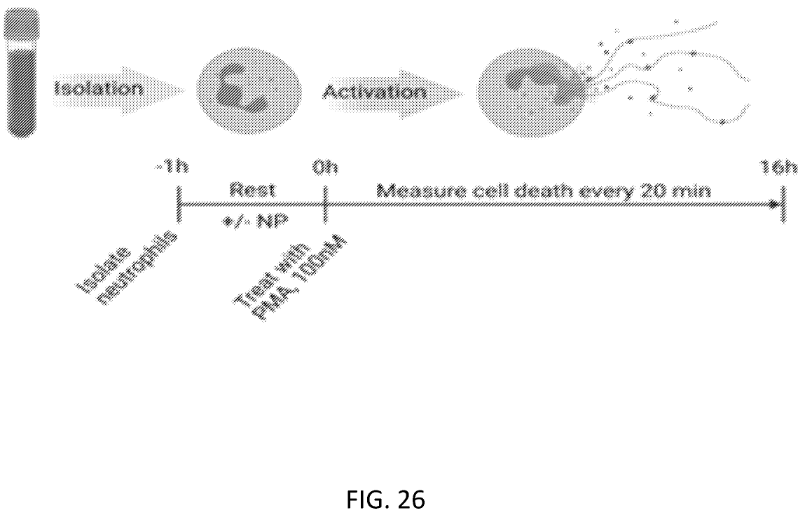

The effect of Effect of exemplary nanoparticles on TNF-alpha in LPS-activated macrophages was assessed as follows: THP-1 cells were seeded in a 24 well plates at a density of 500,000 cells/500 ul media in each well. Cells were differentiated using 10 ng/ml of Phorbol-12-myristate-13-acetate (PMA) and activated using lipopolysaccharide (LPS) at 1 μg/mL. The cells were incubated for 24 hours with 75 μg/mL exemplary nanoparticles, control nanoparticles lacking ligand, After 24 hours, supernatants were collected and assayed for flu TNF-alpha by ELISA (R&D systems). FIG. 26 reflects the results, graphs show significant inhibition of TNF-alpha protein levels in LPS activated cells 24 hrs post treatment with PSA-PLGA when compared LPS alone **p=0.006 and *p=0.01 between PSA-PLGA LPS treated and Blank-PLGA and LPS using Sidak's multiple comparisons test.

### IX. Example 14: Effect of Exemplary Nanoparticles on VEGF Secretion in LPS-Activated Macrophages

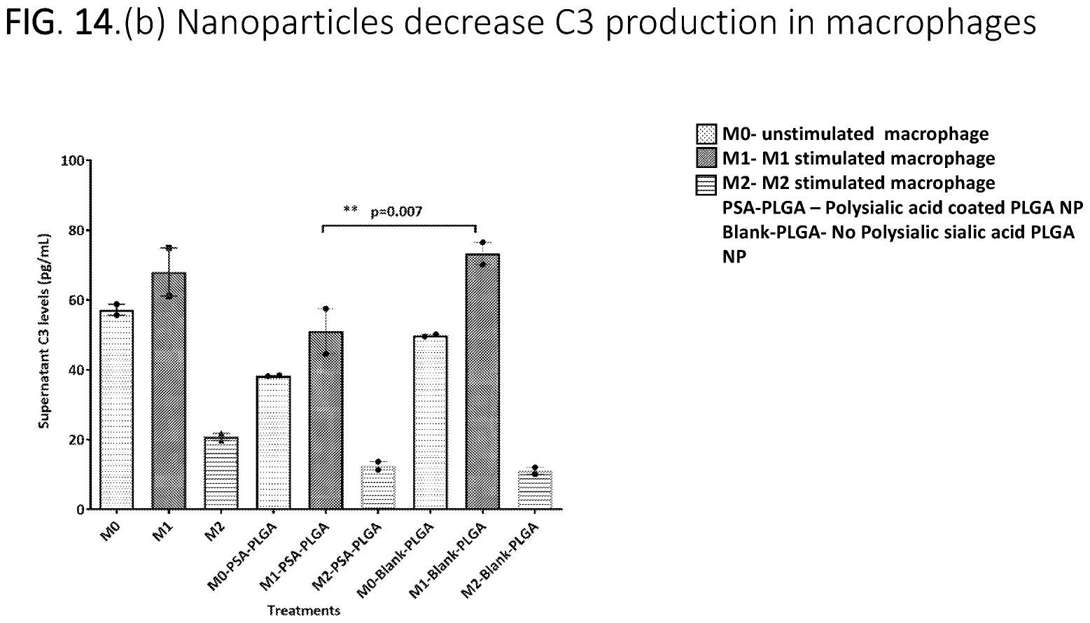

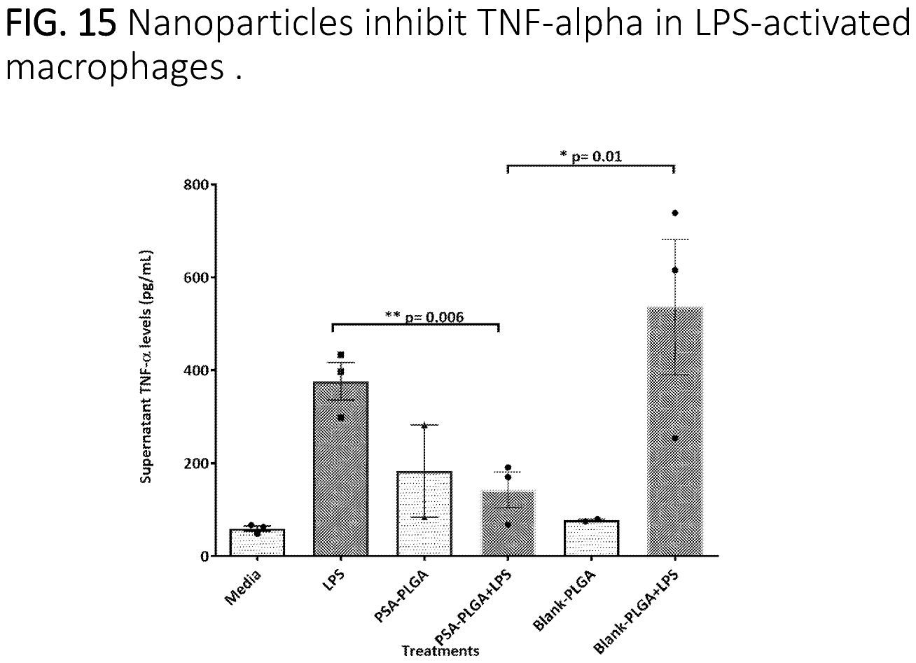

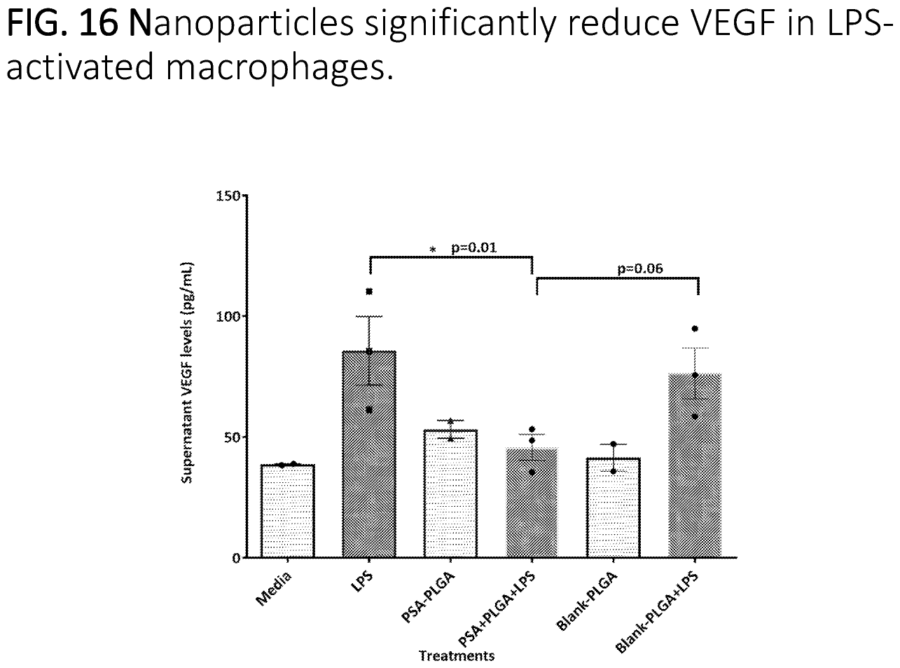

The effect of exemplary nanoparticles on VEGF secretion in LPS-activated macrophages was assessed as follows: THP-1 cells were seeded in a 24 well plates at a density of 500,000 cells/500 μL media in each well Cells were differentiated using 10 ng/mL of phorbol-12-myristate-13-acetate (PMA) and activated using lipopolysaccharide (LPS) at 1 μg/mL. The cells were incubated for 24 hours with 75 μg/mL of exemplary nanoparticles, control nanoparticle lacking ligand, After 24 hours, supernatants were collected and assayed for Hu VEGF levels by ELISA (R&D systems). Graphs represents significant inhibition of VEGF protein levels in LPS activated cells 24 his post treatment with PSA-PLGA when compared LPS alone *p:=0.01 and p=0.06 between PSA-PLGA LPS treated and Blank-PLGA and LPS using Sidak's multiple comparisons test FIG. 15 reflects that the exemplary nanoparticles inhibit VEGF levels in LPS-activated macrophages.

### Pharmacological Experiments and Methods

The HALOS (High Affinity Ligand for Siglec) platform consists of a nanoparticle comprised of a polyethylene-glycol/polylactic-glycolic acid core decorated with natural and synthetically modified sialic acids on its surface. The reasoning behind this chemical structure is several fold. First, the nanoparticle core provides a stable yet biodegradable scaffold for the presentation of the naturally and synthetically modified sialic acids to the Siglec receptors present on immune cells. Second, it is believed that the presentation of a relatively high density of these ligands to the immune cells will augment the potency of the anti-inflammatory signaling through enhancement of avidity, that is, strong cell binding through a multiplicity of weak interactions. Natural and synthetic modification of the sialic acid is aimed at increasing affinity of the individual ligands to the targeted Siglec receptor to also augment the potency of the nanoparticle toward anti-inflammatory signaling.

The strategy discussed herein addresses severe chronic “nonresolving” inflammation in the eye and all inflammatory diseases. Instead of blocking the cytokines produced by inflammatory cells or one inflammatory pathway in a given cell, our nanoparticle formulation utilizes the activated immune cells natural shut down mechanism. This shutdown mechanism involves the binding of a Self-Associated Molecular Patterns (SAMPs) to a Self-Associated Pattern Recognition Receptor (SPRR) that in turn converts an immune cell's phenotype from pro-inflammatory to anti-inflammatory. The agonism of SPRRs by sialic acid immunoglobulin-like lectin (Siglecs) is the immune system's innate inflammatory resolution mechanism.

In animal systems studied by others, short chain sialic acid polymers appear to attenuate inflammation by binding to immunoglobulin-like lectin 11 (Siglec 11) receptors (Karlsletter, 2017) and inducing biologic responses, which upregulate anti-inflammatory cytokines like IL-10, and downregulate pro-inflammatory signals such as reactive oxygen species (ROS), and cytokines TNF-α, IL-12, and VEGF. The activation of Siglec signaling ultimately intersects with the complement alternative pathway (Gao, 2015; Akhtar-Schafer, 2018; Cascella, 2014; Chan, 2014; Jager, 2007; Killingsworth, 1990; Kelly, 2007; Kauppinen, 2020; Zhou, 2017; Zhao, 2013).

The approach described herein is to selectively repolarize the M1-type macrophages that phagocytose photoreceptor outer segments in the retina during disease by converting them to M2c-type macrophages also known as the “resolution macrophage” (Lurier, 2017). The route of administration for our nanoparticle formulation is one or more of intravenous, intravitreal, oral, intraocular, subretinal, subcutaneous, intrascleral, periocular, inhalational nasal and oral, intramuscular, intra-arterial, intraspinal, intrathecal, trans-tracheal, intracranial, intradermal, transdermal (topical), transmucosal, subcutaneous, pulmonary lavage, gastric lavage, and intrahepatic, subcutaneous, or rectal to patients with severe chronic/acute “non-resolving” inflammation in several inflammatory diseases. The particles can function in two ways. (1) down-regulation of complement mediated inflammatory pathways and (2) reprogramming inflammatory macrophages that contribute to pathology. Clinical and experimental evidence from others has indicated that these two pathways are deranged in patients with age-related macular degeneration and other inflammatory disease and can be amenable to therapy. Neumann group (Karlsletter 2017) has shown that Polysia is a sialic acid polymer with a2-8 linkages. It is known to bind Siglec 11 on macrophages. This binding of Siglec 11 provides its anti-inflammatory effect against macrophages, monocytes and microglial cells in the eye which was demonstrated in eyes treated with polysia in a laser CNV model. In this same model, Vascular leakage was also attenuated at the same level as using anti-VEGF agents.

In vitro pharmacology studies have been performed to confirm the mechanism of action of particles described herein in different formulation batches (such as AT-007-NP01-06) in macrophages derived from THP-1 cell lines and macrophages (M1 and M2) derived from normal human peripheral blood mononuclear cells (PBMC), fibrocytes, and neutrophils.

Analysis of the expression patterns of these cells with enzyme-linked immunosorbent assay (ELISA) suggests that AT-007-NP01-06 modulates the production of cytokines, cell death, fibrocyte differentiation and components of the complement factor pathway.

A series of in vitro studies to demonstrate the anti-inflammatory properties of AT-007-NP01 through 06 in macrophages derived from 2 cell types. (1) LPS-activated THP-1 cell line (ATCC TIB-202™, Gaithersburg, MD) and (2) primary human peripheral blood mononuclear cells (PBMC; Stem Cell Research, Cat No-70500.2) (3) PBMC derived Neutrophils (4) Monocytes differentiated fibrocytes were performed using 1 or more AT-007-NP01 through 0 nanoparticle preparations identified as formulation number (see Tables 4 and 8, above).

Table 9 describes the experiments conducted

TABLE 9

| | | | | | AVD-104 |
|---|---|---|---|---|---|
| | Cell Type | Assay | Study design | Results | batch No. |
| 1 | | Ex vivo | RNA and | Diseased ocular tissues | — |
| | | | protein assays | have higher expression of | |
| | | | | Siglec 7, 9, 11 | |
| 2 | | Binding | ELISA based | AT-007-NP04 exhibited | AT-007-NP04 |
| | | assay | | binding to Siglecs 7, 11, 9 | |
| | | | | compared to control Fc | |
| | | | | IgG and blank NPs. | |
| 3 | Macrophages | SHP-1 | Western blot for | AT-007-NP02 exhibited | AT-007-NP02 |
| | (M1) derived | recruitment | SHP-1 protein | SHP-1 recruitment in LPS | |
| | from normal | | after AT-007- | treated THP-1 | |
| | PBMC | | NP02 | macrophages and M1 | |
| | | | | macrophages. | |
| 4 | Macrophages | ELISA for | M1 | AT-007 alone is not | AT-007-NP-02 |
| | (M1) derived | TNF-a | macrophages | sufficient to suppress | |
| | from normal | | treated with | TNFa there by conjugating | |
| | PBMC | | LPS, AT-007 | them to nanoparticles | |
| | | | and AT-007-NP | exhibits an increased | |
| | | | 02 | suppression of TNF-a | |
| | | | | response | |
| 5 | Macrophages | ELISA for | M1 | AT-007-NP-04 exhibited | AT-007-NP-06 |
| | (M1) derived | IL-10 | macrophages | dose dependent increase in | |
| | from normal | | treated with LPS | IL-10. | |
| | PBMC | | and different | | |
| | | | doses of AT- | | |
| | | | 007-NP 04 | | |
| 6 | Macrophages | ELISA for | M1 | AT-007-NP-06 exhibited | AT-007-NP-06 |
| | (M1) derived | TNF-a and | macrophages | dose dependent | |
| | from normal | IL-6 | treated with LPS | suppression of TNFa and | |
| | PBMC | | and different | IL-6 from 0.4 mg/ml- | |
| | | | doses of AT- | 2.1 mg/ml AT-007-NP06 | |
| | | | 007-NP 06 | total solids of | |
| | | | | nanoparticles. | |
| | Complement assays | | | | |
| 7 | | CFH binding | Biacore- SPR | AT-007-NP04 enhances | AT-007-NP-04 |
| | | to C3b | | binding of CFH to C3b | |
| 8 | | C3b | Defibrinated | Decreased c3b deposition | AT-007-NP-04 |
| | | deposition | blood serum | on plates co treated with | |
| | | | | AT-007-NP04 | |
| | In vivo studies | | | | |
| 9 | | Tolerability | Mouse ocular | AT-007-NP03 was well | AT-007-NP-03 |
| | | | tolerability | tolerated with no toxicity | |
| 10 | | Ocular | Rabbit | AT-007-NP03 had no | AT-007-NP-03 |
| | | tolerability | tolerability | toxicity and was present in | |
| | | and | | the vitreous till Day 7 post | |
| | | distribution | | intravitreal injection. | |
| 11 | | Dry AMD | Mouse Bright | AT-007-NP02 showed | AT-007-NP-02 |
| | | efficacy | light damage | significant protection of | |
| | | model | | outer nuclear layer, | |
| | | | | reduced macrophage | |
| | | | | infiltration compared to | |
| | | | | AT-007 free ligand alone | |
| | In vitro | | | | |
| | assays | | | | |
| 12 | Fibrocytes | % Fibrocyte | Prophylactic | Treatment with AT-007- | AT-007-NP-04 |
| | derived from | differentiated | treatment of | NP04 showed in trend in | |
| | monocytes | | with AT-007- | reducing the | |
| | | | NP 04 and SAP | differentiation of | |
| | | | for 5 days on | monocytes to fibrocytes. | |
| | | | monocytes | | |
| 13 | Neutrophils | Cell death | Cell death | AT-007-NP06 exhibited | AT-007-NP04 |
| | derived from | assay | kinetic assay | delayed cell death of | |
| | whole blood | | Treatment of | neutrophils when treated | |
| | | | AT-007-NP06 | with PMA | |
| | | | on neutrophils | | |

### IX. Example 15: Ex-Vivo-Pharmacology Studies

The supporting studies presented herein show increased gene expression (qRT-PCR) of Siglecs 7, 9, and 11 in retina-RPE-choroid complex obtained from 3 exudative and nonexudative AMD eyes (1 female and 2 male donors) aged 85+10 years. Exudative AMD donors exhibited an increase in Siglecs 7, 9, and 11 gene expression by respectively 90)-, 64-, and 58-fold compared with normal donors. Similarly non-exudative AMD donors showed an increase in Siglecs 7, 9, and 11 gene expression by respectively, 77-, 32-, and 27-, fold compared with normal donors. Our gene expression and protein levels data from isolated retina-RPE-choroid complex from healthy subjects with and without AMD show upregulation in the disease state providing further support of our selection of Siglec receptors as a target to treat nonexudative AMD patients. Table 10 summarizes the findings.

TABLE 10

| | Exudative AMD | | Non exudative AMD | |
|---|---|---|---|---|
| | (fold increase relative | | (fold increase relative | |
| | to normal donors) | | to normal donors) | |
| Siglec | Gene | Protein | Gene | Protein |
| Identity | Expression | Expression | Expression | Expression |
| 7 | 90 | 2.35 | 77 | 4.28 |
| 9 | 64 | 1.44 | 32 | 1.20 |
| 11 | 58 | 2.81 | 27 | 2.10 |

### 1. ELISA Based Binding Affinity of AT-007-NP04

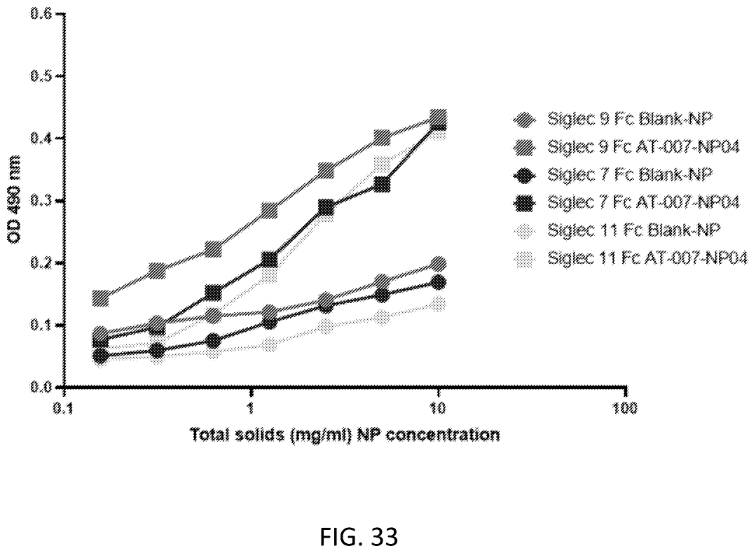

Siglec Fc 7, 9, 11 were coated on ELISA plates and detected using anti PEG Biotin/HRP standard manufacturer's ELISA protocol (R&D Systems). Absorbance was measured at 490 nm. Graphs suggest AT-007-NP04 exhibits higher binding affinity towards Siglec 7, 9 and 11 compared to blank NP controls. See FIG. 33.

### Derivation of THP-1 LPS-Activated Macrophages

The THP-1 cells used in these experiments are a “monocyte-like” cell line derived from a one-year old boy with leukemia (Tsuchiya, 1980). The cells express complement 3 (C3) and Fc receptors. They are phagocytic (for both latex beads and sensitized erythrocytes and others) but lack surface and cytoplasmic immunoglobulin. The cells are weakly responsive to toll-like receptor agonists in their undifferentiated state but become more responsive after differentiation. Cells were grown in Roswell Park Memorial Institute (RPMI) culture medium that had been supplemented with 20% fetal bovine serum (FBS; Gibco, 10438026). The initial seeding and incubation were conducted in T-25 flasks for 2-3 days. At 48 hours before use the differentiation of THP-1 cells to monocytes were induced with 10 ng/mL, phorbol 12-myristate 12-acetate (PMA) in serum-free RPMI. The exact function of the PMA is not known but it is believed to mimic signaling molecules that are inserted in the inner face of the plasma membrane and stimulate the protein kinase C pathway. Thus, once added it cannot be washed away and differentiation is terminal. After an additional 48 hours, the differentiated cells were adherent and ready for activation with 1 μg/mL lipopolysaccharide (LPS). The cells were evaluated for bioactivity using western blot for SHP-1 phosphorylation, cellular binding using IHC and supernatants were used for ELISA based cytokine release assays. (Abbreviations used: PMA=phorbol 12-myristate 13-acetate: LPS=lipopolysaccharides.)

Derivation of M1 and M2 Macrophages from PBMC

In addition to macrophages derived from the THP-1 cell line, we isolated primary human macrophages from fresh, pack Leukopak® (Stemcell Technologies, Cambridge, MA). Primary macrophages are difficult to isolate in sufficient amounts from tissue and do not proliferate in culture. Therefore, we isolated and enriched monocytes from single-donor derived Leukopaks using the “Easy 50” EasySep Magnet™ (Stemcell Technologies; Cambridge, MA) cell separation method. The desired monocytes were enriched with CD14+ magnetic beads and the homogeneity of the population was confirmed using flow cytometry. The CD14+ enriched cells were frozen.

Approximately 6 days prior to use, the cryopreserved monocytes were thawed and cultured in 24-well plates at 250.000 cells/250 μL with serum-free ImmunoCult™—SF Macrophage Differentiation Medium with 50 ng/mL macrophage colony stimulating factor (MCSF). The medium was used to differentiate the monocytes into M1 (classically activated) and M2a (alternatively activated) macrophages. M1 cells were activated by the addition of LPS (10 ng/mL) and interferon-gamma (IFN-γ; 50 ng/mL).

M2 cells were activated by the addition of IL-4 (10 ng/mL). M0 cells were obtained from media without the addition of any activating agents. The cells were evaluated for viability and bioactivity using western blot for SHP-1 phosphorylation, cellular binding using IHC and supernatants were used for ELISA based cytokine release assays and the blank nanoparticle control for cytokine release.

### 2. Activation of Siglec Effectors: SHP1

The initial series of in vitro pharmacology studies demonstrated that different batches of AT-007-NP01 through 06 can mediate biological activity as measured by cytokine release in activated macrophages. More advanced studies were designed to determine if different batches of AT-007-NP01 through 06 binding to the surface of activated macrophages could stimulate intracellular effectors that were consistent with Siglec binding. Siglecs possess intracellular signaling domains that are either inhibitory or activating.

The inhibitory domains are known as immunoreceptor tyrosine inhibitory motifs (ITIMs), while the activating domains are known as immunoreceptor tyrosine activating motifs (ITAMs). Although the Siglecs are quite diverse with the most homology between man and non-human primates, the ITIM and ITAM motifs are conserved across species and represent the immune systems common global activation and deactivation switches for immune cells. When ligands bind to Siglecs the ITIM, is activated and this change recruits Src homology region 2 domain-containing Phosphatase-1,2 (SHP1, SHP2).

SHP1 and SHP2 are protein tyrosine phosphatases (PTPs) that regulate a variety of cellular processes including cell signaling, growth and differentiation, and oncogenic transformation. When SHP1 and SHP2 are recruited, they dephosphorylate the active tyrosine kinases, in particular those found on the ITAMs.

A series of Western Blot experiments was designed to evaluate whether AT-007-NP02 agonizes Siglecs on macrophages and in turn mediate the recruitment of SHP1 and key signaling pathway regulated by SHP1 in myeloid cells. Some of the signaling pathways impacted by SHP1 in macrophages and monocytes include recruitment of SHP1 by ITIM-containing proteins to dampen down ITAM receptor-mediated signaling and negative regulation of signaling through TLRs and cytokine receptors (Abram, 2017). Proteins were resolved by means of gel electrophoresis, blotted, and stained with SHP-1 and beta actin as internal control. Results indicated that after treatment with AT-007-NP02 in M1 activated macrophages recruits SHP-1 indicating AT-007-NP02 plays an agonist role in inhibiting inflammatory response. These data provide confidence that the AT-007-NP02 construct is an agonist of the Siglec pathway in primary PBMC-derived M1-type and LPS-activated macrophages from THP-1 cell lines.

### Cellular Binding

AT-007-NP-03 was used to interrogate binding in macrophages derived from activated THP-1 human monocyte cell line (THP-1) cell lines and PBMC primary cells. The macrophages were plated in a chamber slide for 24 hours in either RPMI serum free differentiation media (for THP-1 cells) or ImmunoCult-SF Macrophage Dedifferentiation Media with macrophage colony stimulating factor (MCSF) (50 ng/mL) for PBMC-derived macrophages. Cells were then treated with either AT-007-NP-03 (75 μg/mL) or FITC-conjugated Blank-NP for up to 24 hours at 37° C. with 5% CO2. Results suggested that cells treated with AT-007-NP03 showed bright green punctate fluorescence staining indicative of AT-007-NP03 binding and colocalization in clusters on the surface of activated cells while the Blank NP appeared to have nonspecific binding all macrophage cells. The data suggested that FITC-conjugated AT-007-NP03 nanoparticles are not incorporated into the cells but adhere to the outer membrane. The apparent association of AT-007-NP03 with the macrophages seemed specific. The Blank-NP which is devoid of ligand did not associate with the cells. This result is consistent that the AT-007 ligand PSA alone presented by AT-007-NP03 associates with elements on the membrane of theses activated macrophages.

### Effect of AT-007-NP on Cytokine Release

The bioactivity of different formulations of AT-007-NP through Siglec binding on macrophages was explored by measuring cytokine release from activated macrophages.

### 3. Conjugation of Nanoparticles to AT-007 Suppresses Pro Inflammatory Cytokine Response

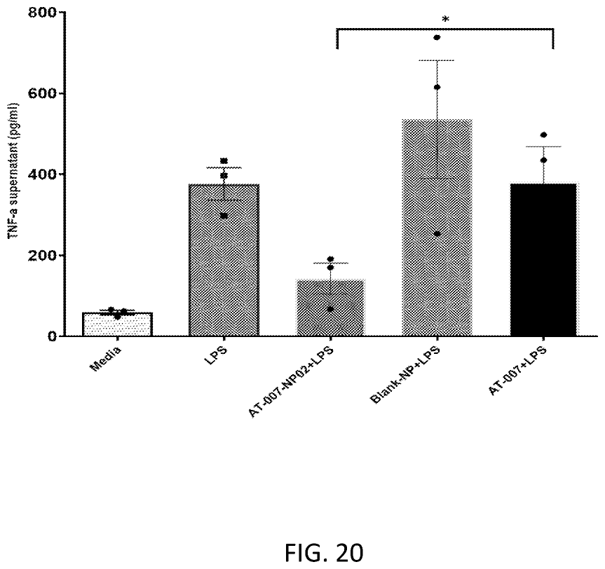

THP-1 cells were differentiated using 10 ng/mL of Phorbol-12-myristate-13-acetate (PMA) and activated using lipopolysaccharide (LPS) at 1 μg/mL. The cells were incubated at 75 μg/ml dose of AT-007-NP02 and AT-007 and blank NP for 24 hrs. After 24 hrs supernatants were collected and assayed for Hu TNF-alpha by ELISA (R&D systems). Graphs shown in FIG. 20 represent significant inhibition of TNF-alpha protein levels in LPS activated cells 24 hrs post treatment with AT-007-NP02 when compared AT-007 alone *p=0.05. Below graph shows that the presence of AT-007 (PSA alone is not sufficient to suppress TNF-α response after LPS treatment on macrophages, adding supporting evidence on conjugating ligands to NP to increase the biological response in these cells.

### 4. AT-007-NP04 Demonstrated Significant Upregulation of Anti Inflammatory Mediator IL-10 in Activated M1 Macrophages

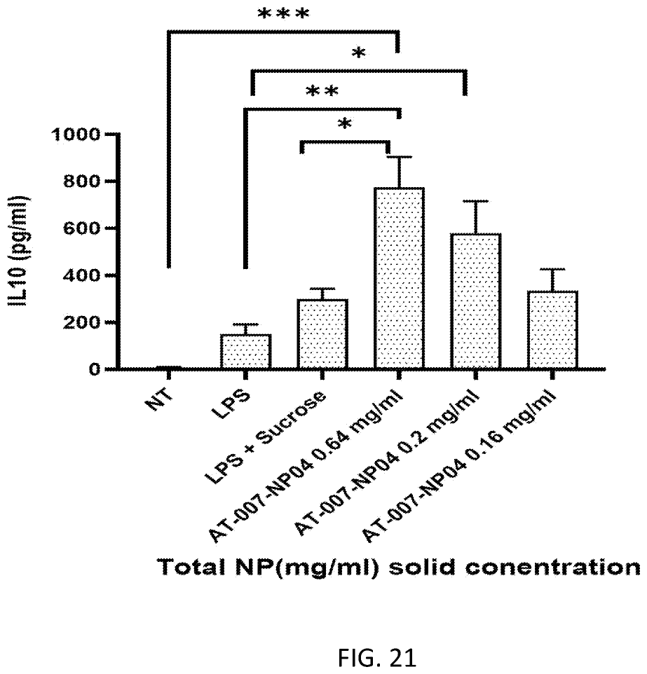

The PBMC were obtained from healthy donors and were activated to M1 phenotype (Hu IFN-y 50 ng/mL. LPS 10 ng/mL) Cells were treated with serial dose range of AT-007-NP04, sucrose (vehicle control) with LPS (100 ng/ml) overnight. Post treatment supernatants were collected are assayed for IL-10 by ELISA (R&D systems following manufactures instruction) Graphs shown in FIG. 21 represents protein levels showing dose dependent increase in anti inflammatory mediator IL-10 16 hrs post treatment with AT-007-NP04 when compared to LPS treated.

### 5. AT-007-NP06 Demonstrated Significant Downregulation of Proinflammatory Mediators IL-6, TNF-a in Activated M1 Macrophages

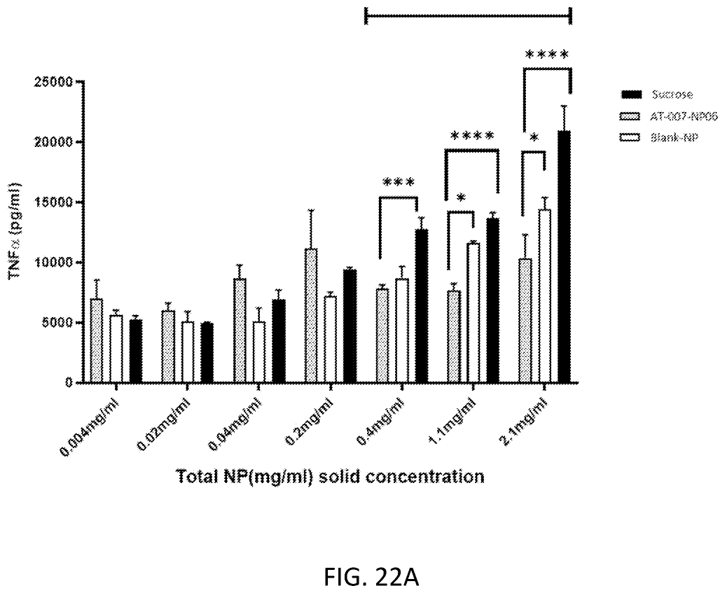

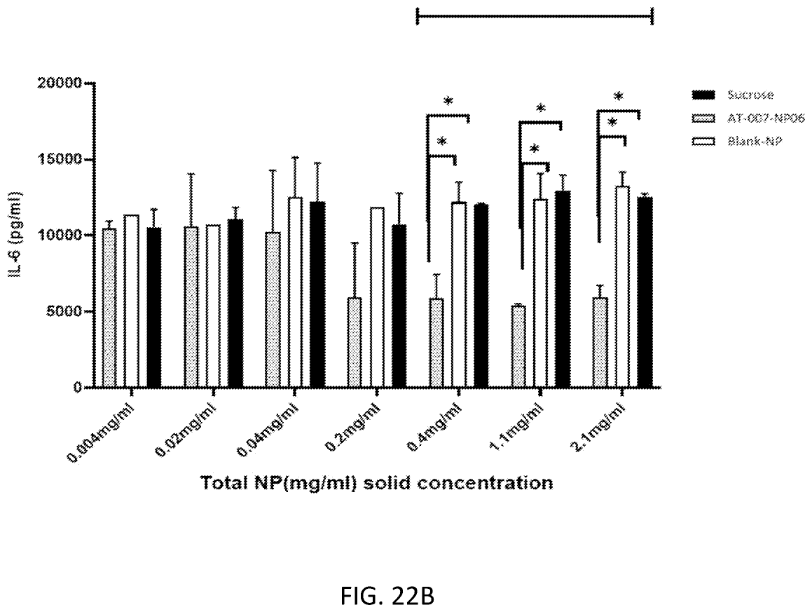

The PBMC were obtained from healthy donors and were activated to M1 phenotype (Hu IFN-y 50 ng/mL, LPS 10 ng/mL) Cells were treated with serial dose range of AT-007-NP06, blank NP and with LPS (100 ng/ml) overnight. Post treatment supernatants were collected are assayed for (A) TNF-α and (B) IL-6 and by ELISA (R&D systems) Graphs shown in FIG. 22A and FIG. 22B represent protein levels showing down regulation of proinflammatory mediators IL-6 and TNF-α at doses 0.4 mg/ml to 2.1 mg/ml 16 hrs post treatment with AT-007-NP06 when compared to blank-NPs. Using turkey multiple comparison ***p:=0.0005, ****p<0.0001, *p<0.03.

The release of human IL-10, TNF-α, IL-6 was quantified by ELISA. All assays were conducted after cells had been incubated in an activating medium overnight and exposed to different concentrations of AT-007-NP06 and AT-007-NP04. The data includes a range of AT-007-NP06 formulation and doses to investigate the biologic activity of nanoparticles. The data indicates that formulation AT-007-NP06, in which the reducing end of PSA is presented to differentiated M1 macrophages, elicits a response that is characteristic of regenerative macrophages in vivo and could potentially be useful in dampening the inflammatory cascades of diseases and increasing the anti-inflammatory response.

Complement components constitute a complex network of about 30 plasma- and membrane-associated serum proteins, designated by numerals (C1-C9) or letter symbols (e.g., complement factors H, FH), which are organized into hierarchal proteolytic cascades. The activation of complement system involves three proteolytic cascades, namely, the classical, lectin, and alternative pathways, which lead to the activation of C3 convertase, the convergence point of all complement pathways. Complement Factor H is also locally produced by RPE and contributes to C3 convertase decay, preventing the amplification of C3b deposition (Geerlings, 2016). Multiple lines of evidence have demonstrated that complement dysregulation, especially the alternative pathway, is involved in the pathogenesis of AMD. Over activation of the complement system have been linked to multiple factors contributing to AMD pathogenesis, such as aging, smoking, and oxidative stress. Nozaki et al. (2006) supported this fact by immune-histological and proteomic studies, which identified complement components as constituents of drusen, suggesting the local activation of the complement pathways. Scholl and many others have shown that in peripheral blood of AMD patients, there is a significant increases in the levels of activated complement components released during the complement activation (Scholl, 2008).

C3b was immobilized on to Biacore plates and complement factor H with or without AT-007-NP06 was flown through into solution and the change in refractive index was measured in real time. The graph shown in FIG. 32B is plotted as response of resonance units (RUs) versus time (a sensorgram). The result demonstrates an enhanced effect of AT-007-NP04 on complement factor H binding to C3b.

Table 11 shows the experimental set up.

TABLE 11

| Ligand | Analyte | Concentration |
|---|---|---|
| C3b (950 RU) | Complement Factor H | 100 nM |
| C3b (950 RU) | AT-007-NP04 | 130 μg/ml |
| C3b (950 RU) | Complement Factor H + | 100 nM + 130 μg/ml |
| | AT-007-NP04 | |

Decreased C3b Deposition on Plates Co Treated with AT-007-NP06 Compared to Sucrose Controls

Many complement proteins are proteases that are activated by proteolytic cleavage. These proteins are called zymogens. Precursor zymogens are distributed through the body, these are activated at sites of infection. These zymogens activate the complete complement system. In the studies described herein, complement activation was induced in defibrinated blood serum by immunoglobulin IgM for classical pathway activation, LPS for alternative complement activation and zymogen as positive control for complement activation PBS was used as a control. Further C3b deposition was analyzed by enzyme-linked immunosorbent assay to quantify complement activation (Karlstetter, 2017). Turkey multiple comparison test showed a significance between sucrose and AT-007-NP06 ****p-<0.0001 for both activators complement pathway zymogen and IgM.

FIG. 33A shows a schematic of the experimental set-up. FIG. 33B shows the bar plots demonstrating decreased C3b deposition on plates co treated with AT-007-NP06 compared to sucrose controls.

### X. Example 16: In Vivo Pharmacology

In vivo pharmacology studies were conducted in murine model of photoreceptor damage model. The bright light damage (BLD) model causes damage to photoreceptors by upregulation of reactive oxygen species (ROS) and dysregulation of the alternative complement system. After brief exposure, these mice can be expected to lose 80% of their photoreceptor layer. In addition, there is ocular expression of a number of proinflammatory and chemotactic cytokines including interleukin 1β, chemokine (C—C motif) ligand 2, cyclooxygenase-2, and TNFα-genes (Bian, 2016). Ocular conditions such as retinitis pigmentosa, Stargardt disease and nonexudative AMD are characterized by photoreceptor cell death (Bian, 2016). Herein we describe our use of the murine BLD model to investigate the efficacy of AT-007-NP-02 in prevention of photoreceptor degeneration.

### 1. Mouse Ocular Tolerability Study

The objective of this study was to evaluate the ocular tolerability AT-007-NP03. AT-007-NP02 administered by IVT injection in the mouse. In cohort 1, the right eyes of 3, healthy 15-16-week old male C57BL/6JRj mice (Janvier, France) received an IVT injection (2 μL) of FITC-Blank-NP. Fundoscopy was performed immediately and 3 days after injection. Both retinas (6 eyes) were prepared and stained for Iba1. For histopathological and immunohistochemical analysis both eyes from 3 animals were postfixed for four hours in 4% paraformaldehyde in 0.1M phosphate buffer solution, pH 7.4, and used to prepare choroidal and retinal flat mounts. Flat mounts were labelled with rabbit anti-Iba1 (Wako, Cat. #019-19741)

Samples were imaged using a fluorescence microscope (Leica DM6B, Leica Microsystems) and immunoreactivity were quantified by determining immunopositive areas (in mm²) using the image processing software FIJI/ImageJ. Qualitative observations did not show any microglial activation or vascular abnormalities in the injected eyes. Eyes injected with FITC-Blank-NP had a strong fluorescent signal, which was detected immediately after the IVT injection. However, on Day 3 fluorescence was reduced and no abnormalities were observed. In cohort 2, mice received an (ocular examination) 5 days before injection. On Day −2, animals were randomized into treatment groups based on fERG a-wave amplitudes. On injection Day, mice received a single bilateral IVT injection (2 μL) of either: (a) AT-007-NP02: 1.3 μg/μL of AT-007 containing 3.4 μg/μL of total solids), (b) Blank-NP (control) or (c) AT-007 (AT-007; 1.3 μg/μL).

Ocular tolerability was assessed by macroscopic OE and IOP measurements at 4, 24, 48, and 72 hours; and 1 and 2 weeks after dose. Just prior to sacrifice at 2 weeks, fERG, SD-OCT, and detailed fundoscopic OEs were performed. Ocular tissues were collected and processed for H&E staining. The posterior cup of right eyes (OD) was embedded in paraffin and sectioned as 5 μm-thick vertical sections (from superior to inferior). Total retinal thickness was measured using Hematoxylin and Eosin (H&E) stained ocular sections in each of these groups. The thickness of the inner layers of the inner retina was measured as thickness from RGC layer to the top of the inner nucleus layer, while outer nuclear layer (ONL) thickness was measured from the bottom of the outer plexiform layer to the external limiting membrane. RGC—retinal ganglion cell, IPL—inner plexiform layer, INL—inner nucleus layer, OPL—outer plexiform layer, ONL —outer nucleus layer, RPE—retinal pigment epithelium No visible gross morphological changes were found in any of the treatment groups

Further evaluated the ocular tolerability of AT-007-NP03 administered by IVT injection in the mouse. Wherein no visible changes were observed during ophthalmic examination. All pupils had a normal reaction to light. No corneal edema, cataract or signs of haemorrhage were detected Heidelberg spectralis HRA (Heidelberg Engineering) OCT scans were compared from Day 0 to 2 weeks post injection.

### 2. PK and Tolerability Study in Dutch-Belted Rabbit (Biodistribution)

The purpose of this study was to evaluate the biodistribution of FITC conjugated AVD-104 in the serum, ocular tissues, liver, spleen, and kidneys following a single IVT injection to Dutch-Belted rabbits (Kaninfarm, Sweden).

This study had two arms. In one arm, biodistribution was evaluated in tissues (serum, ocular tissues) by histological analysis after treatment with a single IVT dose injection of AT-007-NP-03 (0.412 mg/mL AT-007 and 2.1 mg/mL total solids) in rabbits. In the second arm, tissues were collected for bioanalysis following a single IVT administration of unconjugated AT-007-NP-03 (0.412 mg/mL AT-007 and 2.1 mg/mL total solids). Ophthalmic tolerability was evaluated with funduscopy and intraocular pressure for up to 28 days in both arms.

Three days prior to drug administration, tonometry and blood collection was completed on 5 eyes from 12-month-old male rabbits. On Day 0, AT-007-NP-03 (50 μL) was injected IVT in both eyes. Intraocular pressure (IOP) was measured on Days 7, 14, 21, and 28. At the end of the study, blood serum, both eyes, liver, kidney, and spleen were collected and the tissues were fixed in 4% paraformaldehyde for 48 hours. Sections (5 μm) slides were made for H&E staining and for fluorescein microscopy. One cohort of rabbits was treated with AVD-104 without FITC conjugation. At study Days 0 and 28, serum was collected prior to sacrifice. A veterinary ophthalmologist examined the animals. Results show that a single IVT injection of AT-007-NP-03 was well tolerated. No visible changes were observed during ophthalmic examination. The conjunctiva showed no sign of swelling, discharge or hyperemia. The iris was reactive to light and no abnormal folds were noted. The cornea and lens were transparent. There were no signs of anterior chamber flare in the aqueous nor turbidity in the crystalline lens. There were no inflammatory cells inside the vitreous. The retina was unremarkable with no retinal detachments or hemorrhages observed. A fluorescent signal from IVT injected AVD-104 was observed in the fundus immediately after injection. On study Days 3 and 7, the whole vitreous appeared brighter, possibly because of diffused fluorescent nanoparticles. No fluorescent signal was detected in the vitreous at any later time point (Day 14 or 28). There were no differences between treatment groups in IOP on Days 7, 14, 21, and 28. All values were similar to baseline readings taken at study Day 3. Qualitative examination of rabbit retinas stained with hematoxylin and eosin showed no changes.

AT-007-NP-03 was well tolerated. Rabbit eyes injected IVT with AT-007-NP-03 showed no signs of inflammation, swelling, or abnormal bleeding. FITC labelled nanoparticles were clearly visible immediately after intravitreal injection. On Day 3 and some cases on Day 7, the whole vitreous appeared brighter, likely due to diffused fluorescent NPs. On imaging Days 14 and 28, no NP derived autofluorescence was detected. No fluorescent NPs were detected when acquiring autofluorescence images from vertical sections of the retina collected on Day 28.

Ocular examination revealed that conjunctiva showed no sign of swelling, discharge or hyperemia, Iris was reacting to light and no abnormal folds were noticed, Cornea was transparent, no anterior chamber flare was observed. There were no inflammatory cells inside vitreous and no retinal detachments or haemorrhages were observed. Further representative image of rabbit retina stained with hematoxylin and eosin staining indicating no loss of any cell layer and a well tolerated drug at Day 28.

### 3. Proof of Concept in the Mouse Bright Light Damage Model

The objective of this study was to evaluate the pharmacological efficacy of AT-007-NP-02 on the mouse bright light-induced photoreceptor damage model. 8-week old male bright light damage model BALB/cAnNCrl mice (Charles River, Germany) were randomized into 4 groups
Group 1: uninjected control (n=5)
Group 2: bright light damage+Blank-NP control (1 μL containing 3.4 μg/IL of total solids) (n=11)
Group 3: bright light damage+AT-007-NP-02 (1.3 μg of AT-007 in 1 μL containing 3.4 μg/μL of total solids) (n=11)
Group 4: bright light damage+AT-007 (1.3 μg of AT-007 in 1 μL) (n=11)
Total solids: includes AT-007, PEG, and PLGA

Baseline values for flash electroretinography (fERG) and high-resolution SD-OCT were obtained 5 days before the bright light damage induction. On Day −1, test articles were administered (2 μL) IVT OD and mice were dark adopted. On Day 0, in the morning the pupils were fully dilated by applying a drop of 0.5% tropicamide (Oftan Tropicamid. Santen Oy). The animals were moved into clear plastic cages and exposed to a bright light stimulus (10,000 lux) for four hours. After exposure to continuous bright light the mice were returned to normal light/dark cycle (10 lux; 12 h on/12 h off). On Day 7, animals were re-examined fERG and SD-OCT. On the terminal day of the experiment, the animals were perfused transcardially with 0.9% NaCl solution. Both eyes were collected and frozen unfixed with liquid nitrogen. The tissue was stored in optimal cutting temperature (OTC) compound at 80° C. until used for histology/immunohistochemistry. The eyes were sectioned for slides into 5 μm-thick sections. One set of slides were used for Hematoxylin & eosin (H&E) histological staining. A second set of slides were used to stain against the macrophage cell marker F4/80.

Quantitative data were graphed, analyzed and presented as mean standard deviation (SD). Outlier data points were identified using the ROUT's test with a false discovery rate of 1%. Data were analyzed using the GraphPad Prism software (v8.0.1 GraphPad Software. USA). Internal projection (VIP) SD-OCT B-scan data are presented. Histological analysis results are pending. The SD-OCT B-scans were taken from a VIP centered on the optic nerve head (ONH) and located at −0.4 mm inferior to the ONH, at the ONH and at +0.4 mm superior to the ONH. A 25-point grid was placed on the SD-OCT VIP scans centered on the ONH and the thickness of the various retinal layers (inner retina outer nuclear layer-IS/OS/RPE) was measured for each point using the software.

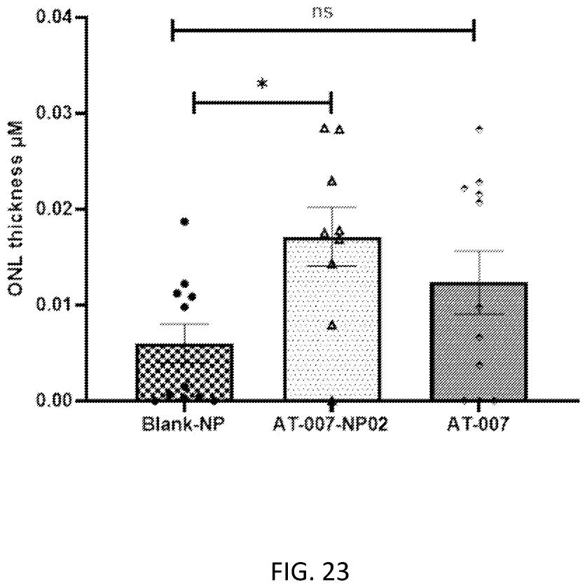

The preservation of the ONL in the ligand alone group with an equivalent amount of AT-007 did not reach statistical significance versus the Blank-NP-treated mice. This suggests that the ligand alone was not sufficient to protect the retina under light damaging conditions. Heat map correlations (not shown) indicate relative changes in the thickness of retinal layers as measured by OCT centered on 3 different regions of the eye. As expected, some regions of the retina are more sensitive to light damage than others. The darker intensity of dark blue squares at the superior nasal orientation (SN) in the AT-007-NP02 treated mice show significant maintenance of the outer nuclear layer (ONL) in that region compared to mice treated with AT-007 only or Blank-NP. The results, shown in FIG. 23, are suggestive that under these extreme conditions of intense bright light, AT-007-NP02 prophylaxis may contribute to the maintenance of the ONL and protect the retina from thinning compared to prophylactic treatment with either AT-007 or Blank-NP alone. This data strongly supports the requirement for the presence of the nanoparticle scaffold to enhance the activity of AT-007 ligand to target and engage the Siglec receptor binding more efficiently in order to elicit an anti-inflammatory/protective response.

FIG. 23 shows Outer nuclear layer thickness (μM) from animals injected with blank-NP, AT-007 and AT-007-NP02. Data is presented as mean+SEM from 11 mice per group Data was analyzed by Sidak multiple comparison Significance Blank-NP vs.AT-007-NP02 * p=0.0318.

The Spectral Domain Optical Coherence Tomography (SD-OCT) retinal scans were used to assess retinal structure, and to analyze outer nuclear layer (ONL). ONL thickness was significantly greater in all analyzed grid points in eyes that received AT-007-NP02 when compared to eyes that were treated with blank-NPs. This difference was also clearly reflected when the data were divided into several anatomical regions of the eye Superior nasal regions had higher protection compared to the temporal regions.

### 4. Effect of AT-007-NP04 on Differentiation of Fibrocytes

PBMC were obtained from healthy donors, CD14+ monocyte cells were isolated and (2×10\hat{ }5/ml) resuspended in serum free fibrocyte differentiating medium and cultured for 5 days. At day 4 the non-adherent cells are washed out and half the medium is removed and replaced with fresh medium. Cells were plated in 24 well plate with and without AT-007-NP04/SAP for bright field imaging and cell counts. Cells were imaged at 10× magnification using Olympus using the FLUOVIEW FV3000 setting. Scale—100 sM. The number of fibrocytes was determined by counting triplicate wells with three fields of view per well. Serum album protein (1 μg/ml) (SAP) was used as a positive control. AT-007-NP06 were treated at a concentration of 50 μg/ml. FIG. 24 depicts the schematics for the experimental setup. Representative bright field images indicated a trend of reducing fibrocyte differentiation post AT-007-NP04 treatment.

The microscopic images clearly showed evidence that upon treatment with AT-007-NP06 we can reduce the differentiation of monocytes to fibocytes. Changes in fibrocyte numbers could correlate to either preventing further pathological fibrosis or may even contribute to its resolution

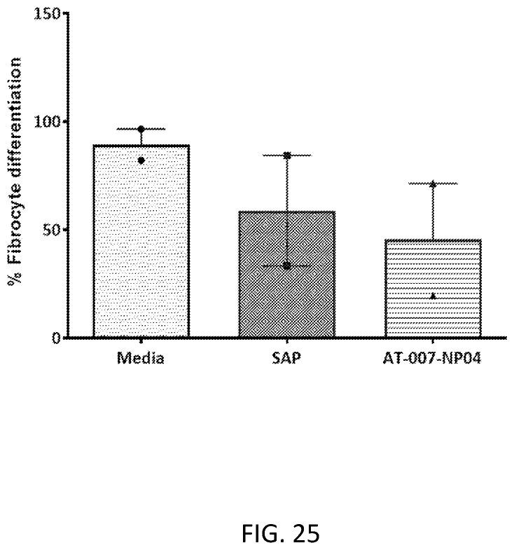

The results are graphical represented in FIG. 25 as % fibrocyte differentiation showing trend in reduction of fibrocyte differentiation based on the counts from the above images.

### 5. Effect of AT-007-NP06 in Delaying NET Formation and Delaying Cell Death Measured Using Sytox Orange Dye

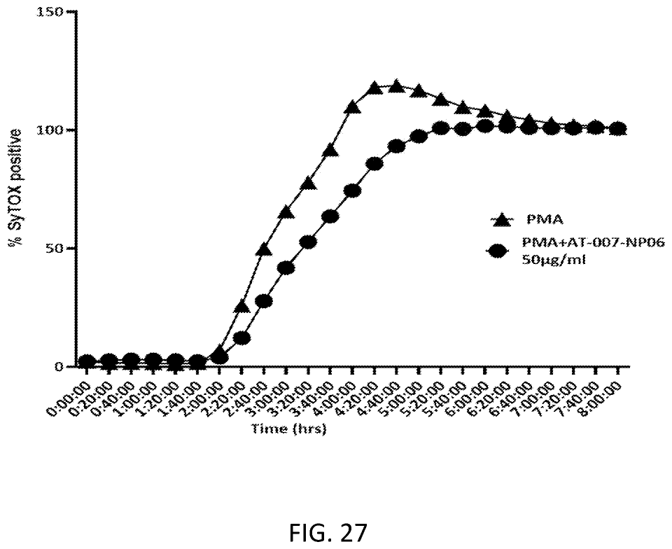

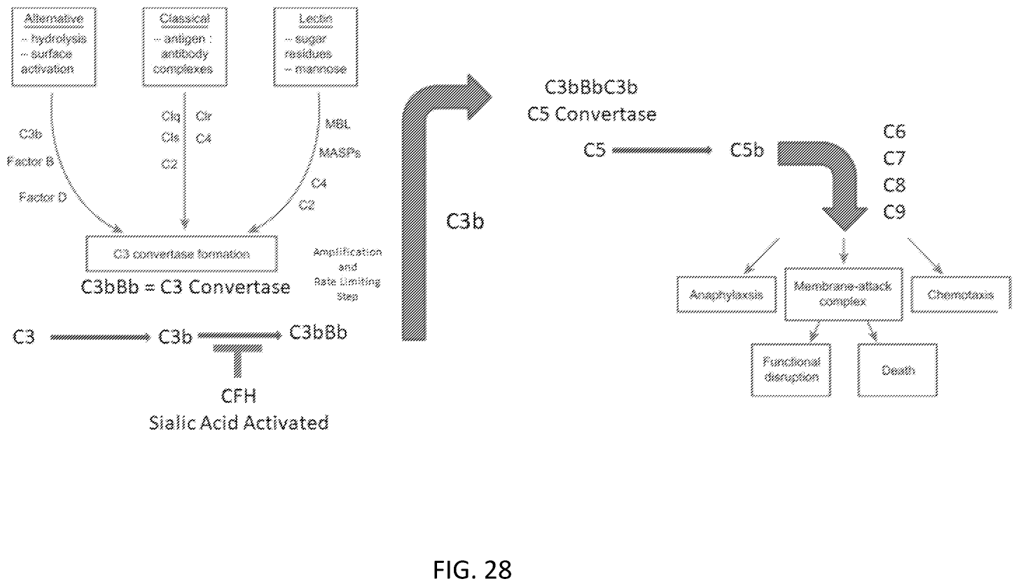

Neutrophils were isolated from the blood of healthy controls using EasySep Human Neutrophil Isolation Kit (StemCell Technologies Catalog #19359. Cells. human PMNs were rested for 1 hr prior to treatment with phorbol myristate acetate (PMA) (100 nM) and 50 μg/ml of AT-007-NP06 along with the presence of the membrane-impermeable DNA-binding dye SYTOX Orange Sytox Orange dye (Thermo Fisher Catalog #—S11368) following the manufacturer's instructions. We observed the extracellular DNA and NET formation over time. Mean SYTOX Orange intensity values derived from these images were used to quantify total extracellular DNA release in wells where PMNs were stimulated with 100 nM PMA as a positive control and nanoparticles. These data demonstrate that in the presence of AT-007-NP06 there was a delay in NET formation compared to PMA controls. The schematic of the experimental set-up is shown in FIG. 26. The results are graphically represented in FIG. 27, which shows the kinetic curve overtime.

Sytox positive is indicative of neutrophil cell death. The higher percentage of Sytox positive staining upon PMA suggests that the cells undergo apoptosis after treatment. Our data suggests that AT-007-NP06 along with PMA treatment delays the neutrophil cell death with time. There is a shift in the curve around 2 hr-4 hr post treatment showing close to 30-50% decrease in neutrophil cell death. This delay in cell death is a strong evidence that our AT-007-NP06 can prevent NETosis.

### XI. Example 17: Binding of Columinic Acid (PSA) to Siglec 7,9. And 11

Binding of polysialyc acid (PSA) molecules of different degrees of polymerization. DP, to Siglec 7, 9, and 11 was investigated using Surface Plasmon Resonance effect (BIACore™ instrument).

It will be understood that the value of DP refers to an average length of polymer molecules in an ensemble of molecules. Thus, PSA with DP 13 refers to an ensemble of PSA molecules having an average length of 13 sialic acid residues. In this experiment, a commercially available dimer of sialic acid (PSA DP 2) was used as a control. This composition comprising this dimer was homogenous and did not have a distribution of lengths.

Results indicate that PSA having DP 13 binds strongly to Siglec 7, 9 and 11, whereas no binding was observed for the dimer of sialic acid (PSA having DP 2).

Briefly, colominic acid was partially depolymerized by heating in water at 80° C. for 2.5 h and fractionated by ultra filtration to obtain a PSA fraction that has an average DP of 13. The compound was modified by oxime ligation to give compound 3

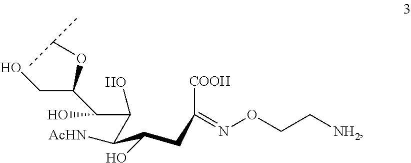

which was modified by biotin (as described below). The wavy line represents the point of attachment to the PSA molecule

### Biotimylation

Biotinylation of the oxime-modified PSA was performed according to the following synthetic scheme:

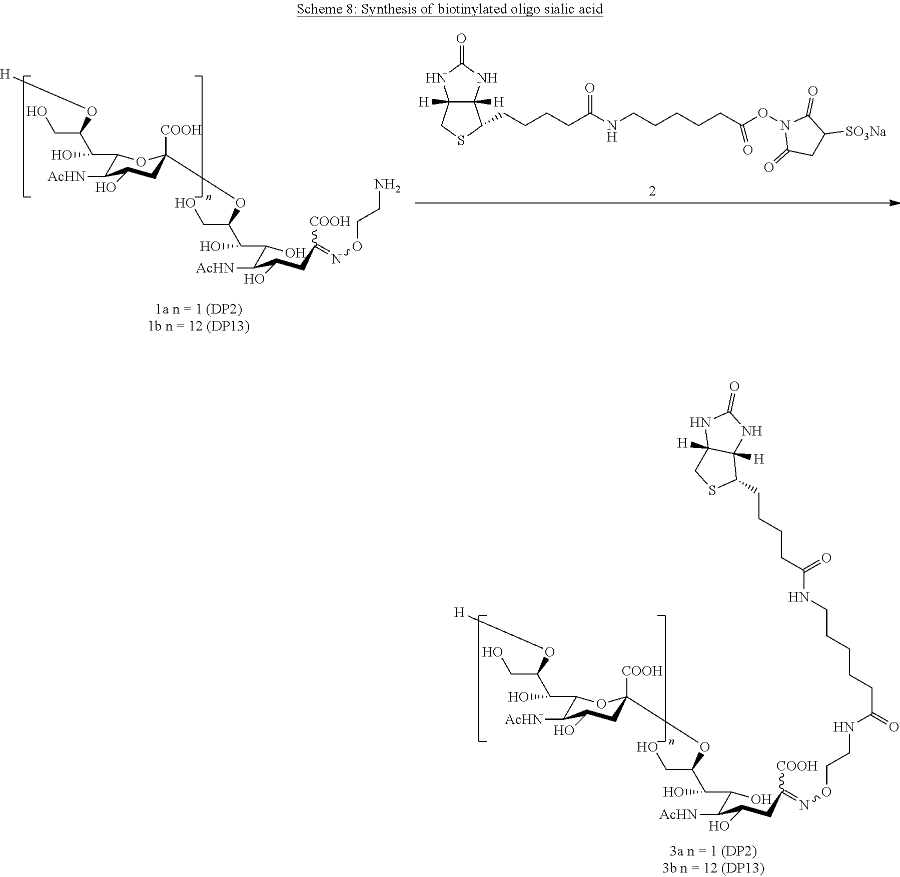

Briefly, to a reaction tube, 1a/1b (1 eq.), H2O, NaH CO3 (1.5 eq.) and compound 2 (1.2 eq.) were added and stirred at room temperature for overnight. The reaction was purified by P2 bio-gel chromatography (3a) or ultrafiltration using 10 K spin filter (3b) to get compound 3a or 3b.

The modified PSA was immobilised to an BIAcore SPR chip modified by Streptavidin and tested for binding to Siglecs.

### Surface Plasmon Resonance (SPR) Analysis of Di- and Polysialosides

### Surface/Sample Conjugation

All analyses were performed on a GE Biacore T100 machine. To a Series S CM5 chip (Cytiva No. 29149603) is coated streptavidin (2,000 response units, Thermo Fisher Scientific No. 434301) using standard EDC/NHS amide conjugation chemistry (Flow cell 1, 2, 3, and 4). All flow cells are subsequently blocked using ethanolamine and the chip surface is stabilized by flowing (10 μL/min) a degassed solution of PBS—P (10 mM Na₂HPO₄, 1.8 mM KH₂PO₄, 137 mM NaCl. 2.7 mM KCl, and 0.5% surfactant P20 (CytivaNo. BR100054), pH 7.4) overnight.

To the stabilized baseline is introduced a biotinylated disialoside and a biotinylated polysialoside with an average degree of polymerization of 13 (DP13). The disiaoloside is dissolved in Dl water to a concentration of 50 μg/mL (Volume total=200 μL). Using the T100 software in manual mode, the 50 μg/mL solution of biotinylated disialoside is introduced to flow cell 2 at a flow rate of 30 μL/min for 90 seconds followed by PBS—P for 90 seconds at a flow rate of 30 μL/min. This process is repeated three times, after which, no additional biotin conjugation is observed. The stabilization of biotinylated polysialoside (DP13) follows the same protocol as the disialoside with the exception that the biotinylated DP13 structure is introduced into flow cell 4.

### Siglec-11 Screening

Lyophylized hFc-Siglec-11 (R&D Systems No. 3258-SL-050) is reconstituted in PBS—P buffer to a concentration of 200 μg/mL and allowed to equilibrate on ice for 30 mins prior to analysis. Parallel binding assays are set up to measure the interaction between siglec-11 and biotinylated disialoside and polysialoside by subtracting the response units measured from flow cell 2 and 1, and flow cell 4 and 3, respectively. This analysis is accomplished by introducing siglec-11 at three different concentrations: 200 μg/mL, 100 μg/mL, and 50 μg/mL using the following parameters:
a. Flow rate=30 μL/min
b. Contact time=60 s
c. Dissociation time 90 s
d. Stabilization time=30 s

### Siglec-9 Screening

Lyophylized hFc-Siglec-9 (R&D Systems No. 1139-SL-050) is reconstituted in PBS—P buffer to a concentration of 200 μg/mL and allowed to equilibrate on ice for 30 mins prior to analysis. Parallel binding assays are set up to measure the interaction between siglec-9 and biotinylated disialoside and polysialoside by subtracting the response units measured from flow cell 2 and 1, and flow cell 4 and 3, respectively. This analysis is accomplished by introducing siglec-9 at a concentration of 100 μg/mL using the following parameters:
a. Flow rate=30 μL/min
b. Contact time=60 s
c. Dissociation time 90 s
d. Stabilization time=30 s

### Siglec-7 Screening

Lyophylized hFc-Siglec-7 (R&D Systems No. 1138-SL-050) is reconstituted in PBS—P buffer to a concentration of 200 μg/mL and allowed to equilibrate on ice for 30 mins prior to analysis. Parallel binding assays are set up to measure the interaction between siglec-7 and biotinylated disialoside and polysialoside by subtracting the response units measured from flow cell 2 and 1, and flow cell 4 and 3, respectively. This analysis is accomplished by introducing siglec-7 at a concentration of 100 μg/mL using the following parameters:
a. Flow rate=30 μL/min
b. Contact time=60 s
c. Dissociation time 90 s
d. Stabilization time=30 s

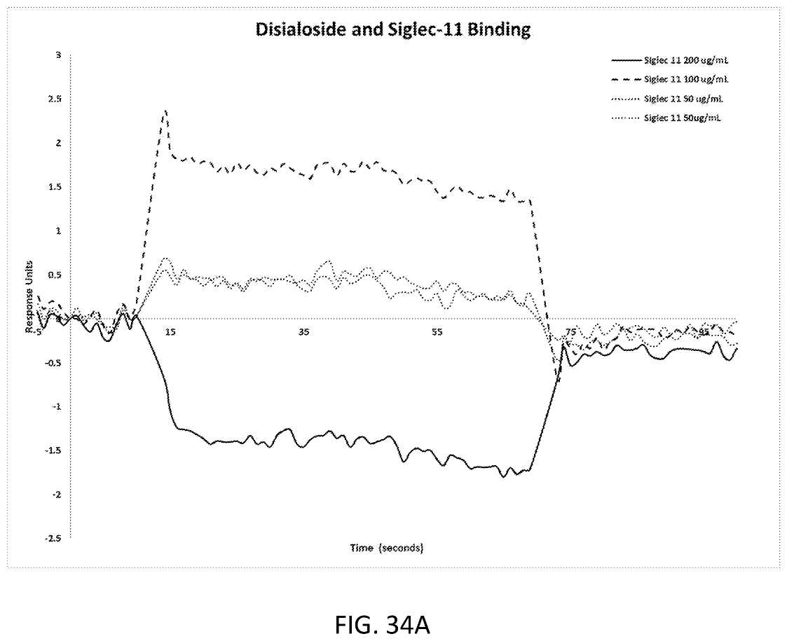

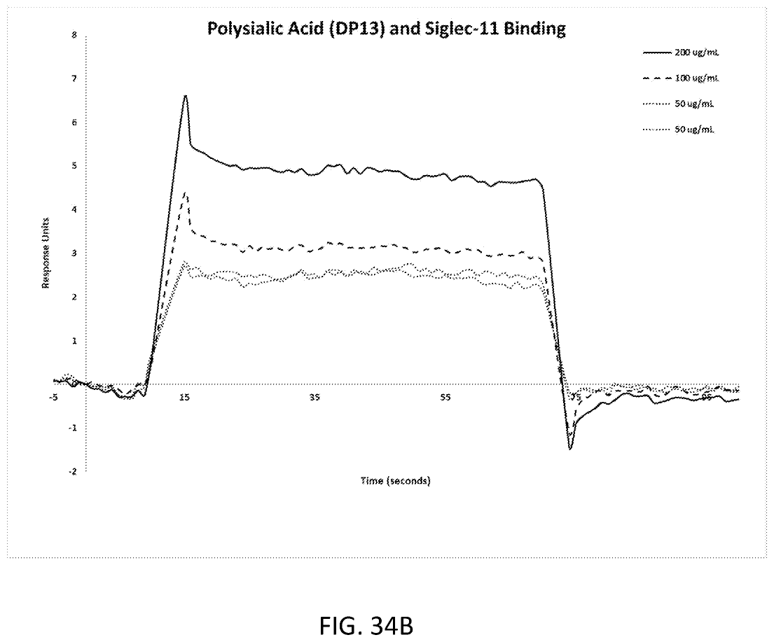

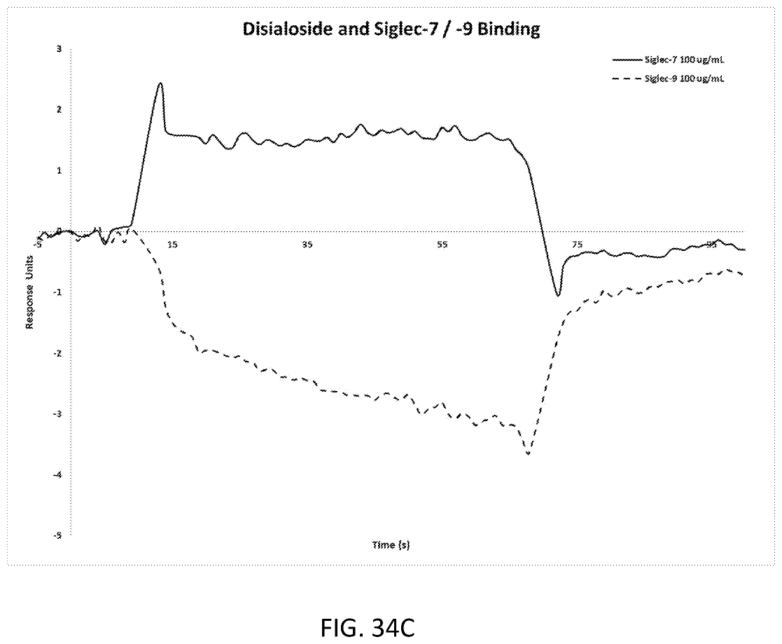

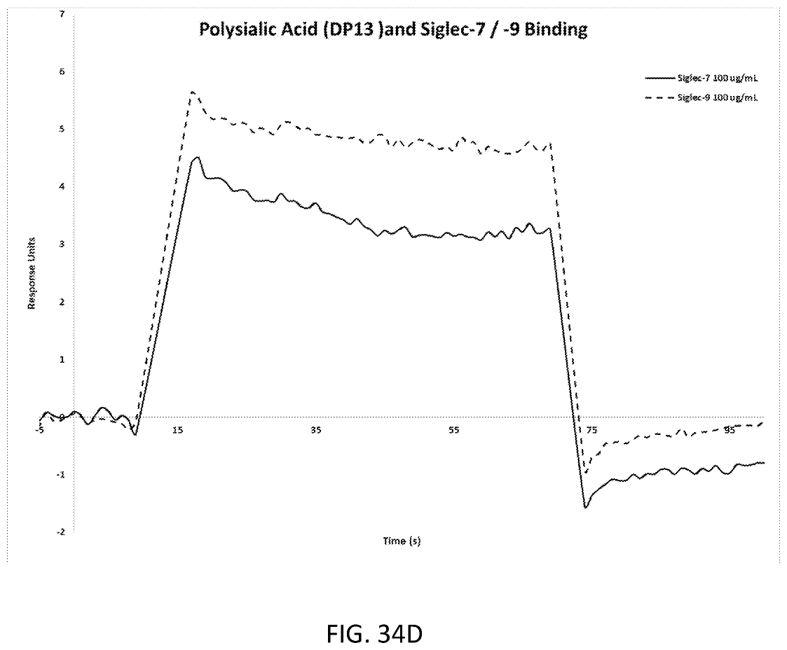

The results are presented in the sensograms shown in FIG. 34A through FIG. 34D. As can be seen, whereas PSA having DP 13 binds to Siglecs 7, 9 and 13, no binding is detected for the disilanyl dimer. These results suggest that the functionality of biding to Siglec depends on the PSA length

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A particle, comprising a molecule represented by the following structural formula:
P-L-G,
wherein:
P is a biocompatible polymer scaffold comprising a block copolymer PLGA-PEG;
G is a polysialic acid (PSA) comprising from 5 to 200 repeat units of sialic acid; and
L is a covalent linker,
or a pharmaceutically acceptable salt thereof.

2. (canceled)

3. The particle of claim 1, wherein P is represented by the following structural formula: 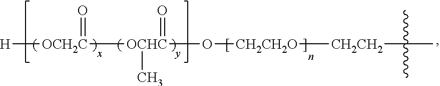
wherein the symbol [symbol] represents the point of attachment of the polymer to the linker L, and further wherein:
x is an integer from 0 to 20, y is an integer from 0 to 20, m is an integer from 1 to 1000, and n is an integer from 5 to 450, provided that x and y are not simultaneously 0.

4. The particle of claim 3, wherein x is an integer from 0 to 10, y is an integer from 0 to 10, m is an integer from 1 to 500, n is an integer from 5 to 450.

5. The particle of claim 1, wherein G is represented by any one of the following structural formulas: 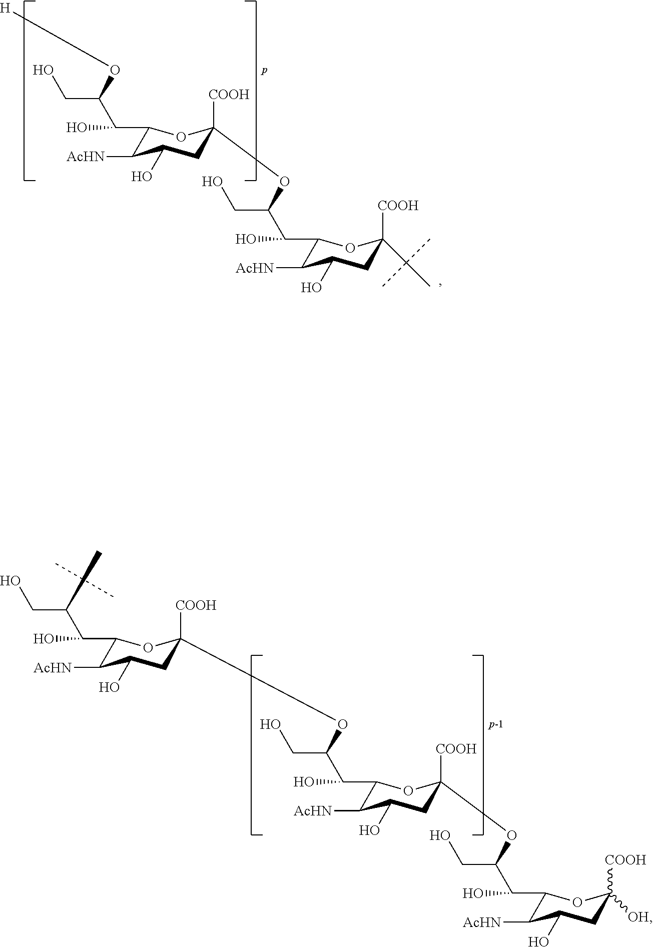
or a pharmaceutically acceptable salt thereof,
wherein the symbol [symbol] represents the points of attachment of G to the linker L, and further wherein p is an integer from 4 to 198.

6. The particle of claim 5, wherein the value of p is selected from any one of the following ranges: from 10 to 20, from 20 to 30, from 30 to 40, from 40 to 50, and from 50 to 60.

7. The particle of claim 5, wherein p is from 5 to 25.

8. The particle of claim 5, wherein p is from 10 to 20.

9. The particle of claim 1, wherein the linker L is represented by any one of the following structural formulas, wherein the symbol [symbol] represents the point of attachment of the linker L to G, and the symbol [symbol] represents the point of attachment of the linker L to P: 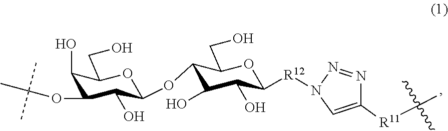
wherein:
R¹¹ is —C(O)NH— or —CH₂—NH—C(O)—CH₂—O—; and
R¹² is absent or is any one of —O—(CH₂)₁₋₁₀—, —(O—CH₂CH₂)₁₋₁₀—, —N(X¹¹)—(CH₂)₁₋₁₀—, —N(X¹²)—O—(CH₂)₁₋₁₀—, or —NHNH—(CH₂)₁₋₁₀—, wherein X¹¹ is H or acetyl, and X¹² is H or methyl; 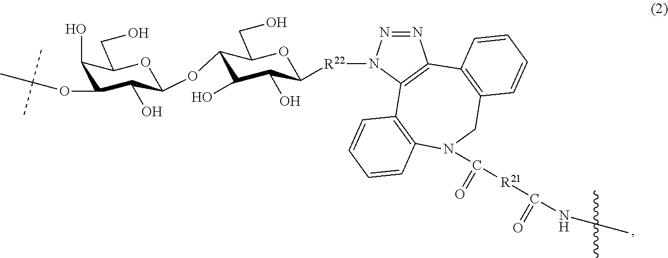
wherein:
R²¹ is —(CH₂)₁₋₁₀— or —(CH₂CH₂—O)₁₋₁₀—(CH₂)₁₋₁₀—; and
R²² is absent or is any one of —O—(CH₂)₁₋₁₀—, —(O—CH₂CH₂)₁₋₁₀—, —N(X²¹)—(CH₂)₁₋₁₀—, —N(X²)—O—(CH₂)₁₋₁₀—, or —NHNH—(CH₂)₁₋₁₀—, wherein X²¹ is H or acetyl, and X²² is H or methyl; 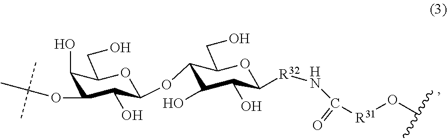
wherein:
R³¹ is —(CH₂)₁₋₁₀—; and
R³² is absent or is any one of —O—(CH₂)₁₋₁₀—, —(O—CH₂CH₂)₁₋₁₀—, —N(X³¹)—(CH₂)₁₋₁₀—, —N(X³²)—O—(CH₂)₁₋₁₀—, or —NHNH—(CH₂)₁₋₁₀—, wherein X³¹ is H or acetyl, and X³² is H or methyl; 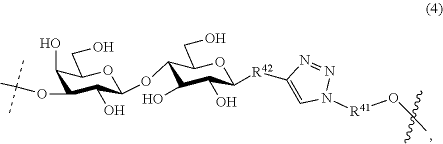
wherein:
R⁴¹ is —(CH₂)₁₋₁₀—; and
R⁴² is absent or is any one of —O—(CH₂)₁₋₁₀—, —(O—CH₂CH₂)₁₋₁₀—, —N(X⁴¹)—(CH₂)₁₋₁₀—, —N(X⁴²)—O—(CH₂)₁₋₁₀—, or —NHNH—(CH₂)₁₋₁₀—, wherein X⁴¹ is H or acetyl, and X⁴² is H or methyl; 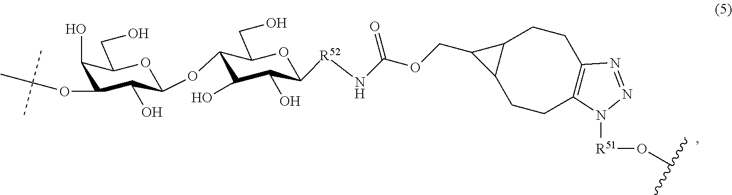
wherein:
R⁵¹ is —(CH₂)₁₋₁₀—; and
R⁵² is absent or is any one of —O—(CH₂)₁₋₁₀—, —(O—CH₂CH₂)₁₋₁₀—, —N(X⁵¹)—(CH₂)₁₋₁₀—, —N(X⁵²)—O—(CH₂)₁₋₁₀—, or —NHNH—(CH₂)₁₋₁₀—, wherein X⁵¹ is H or acetyl, and X⁵² is H or methyl; 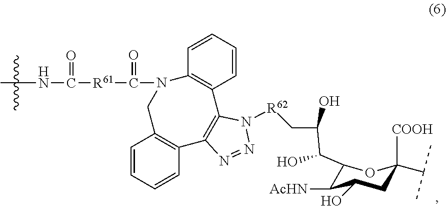
wherein:
R⁶¹ is —(CH₂)₁₋₁₀— or —(CH₂CH₂—O)₁₋₁₀—(CH₂)₁₋₁₀—;
R⁶² is absent, or is —(CH₂)₁₋₁₀— or —(CH₂)₁₋₁₀—O—(CH₂)₁₋₁₀—NH—; 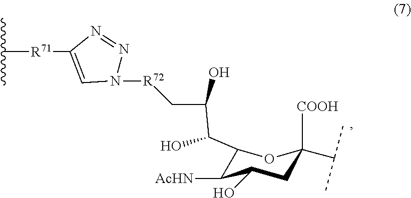
wherein:
R⁷¹ is —NHC(O)— or —OCH₂—C(O)NH—CH₂—; and
R⁷² is —(CH₂)₁₋₁₀—O—(CH₂)₁₋₁₀—NH—; 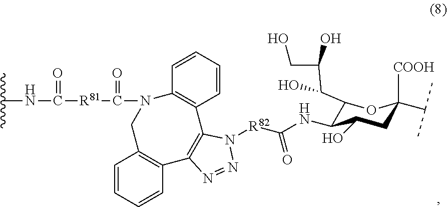
wherein:
R⁸¹ and R⁸², each independently is —(CH₂)₁₋₁₀— or —(CH₂CH₂—O)₁₋₁₀—(CH₂)₁₋₁₀—; 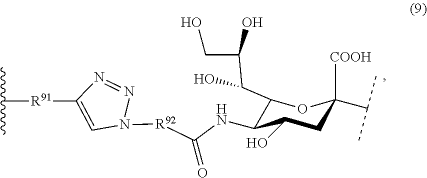
wherein:
R⁹¹ is —NHC(O)— or —OCH₂—C(O)NH—CH₂—; and
R⁹² is —(CH₂)₁₋₁₀— or —(CH₂CH₂—O)₁₋₁₀—(CH₂)₁₋₁₀—; 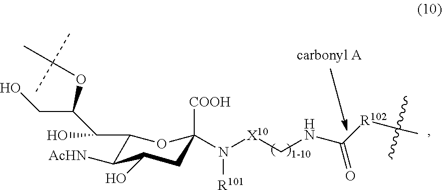
wherein:
R¹⁰¹ is H or methyl;
X¹⁰ is O or NH; and
R¹⁰² is —CH₂O—, or a moiety represented by any one of the following structural formulas: 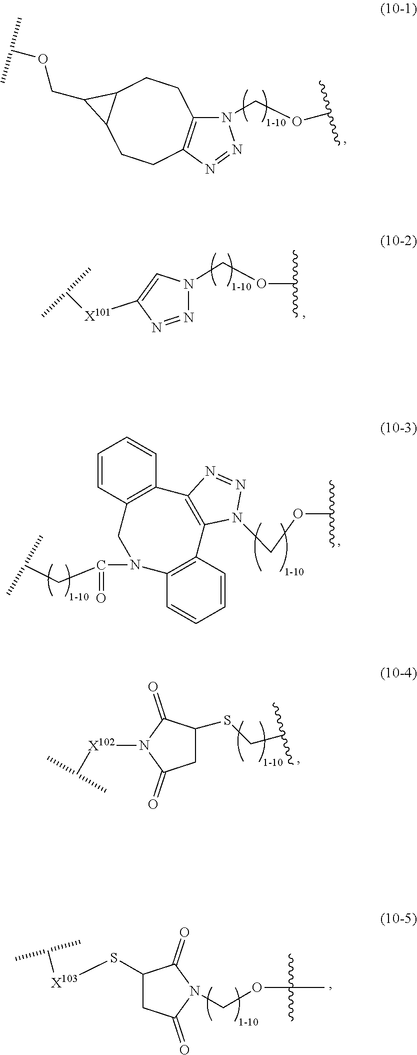
wherein X¹⁰¹, X¹⁰², and X¹⁰³, each independently is —(CH₂)₁₋₁₀— or —CH₂CH₂—(OCH₂CH₂)₁₋₁₀, and wherein the symbol [symbol] represents the point of attachment to the carbonyl A; 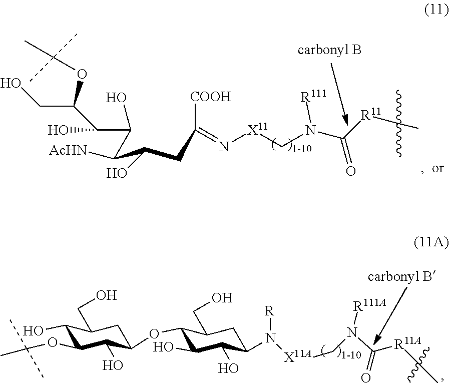
wherein:
R¹¹¹ and R^{111A}, each independently, is H or a C1-C3 alkyl;
X¹¹ and X^{11A}, each independently, is O or NH; and
R¹¹ and R^{11A}, is independently, is —CH₂O—, or a moiety represented by any one of the following structural formulas: 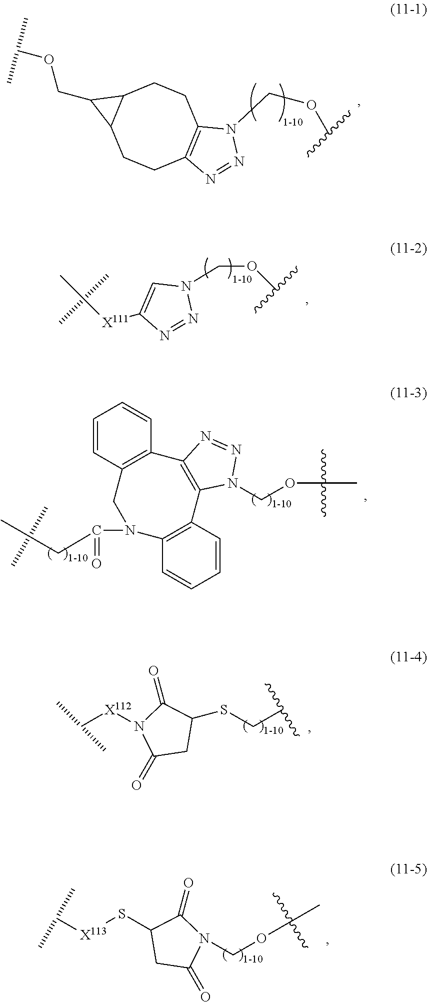
wherein X¹¹¹, X¹¹², and X¹¹³, each independently is —(CH₂)₁₋₁₀— or —CH₂CH₂—(OCH₂CH₂)₁₋₁₀, and wherein the symbol [symbol] represents the point of attachment to the carbonyl B or the carbonyl B′; 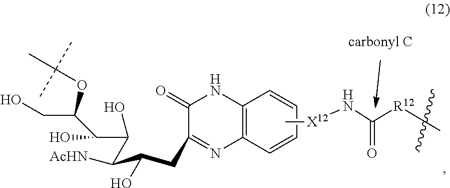
wherein:
X¹² is —(CH₂)₁₋₁₀— or —C(O)—(CH₂)₁₋₁₀—; and
R¹² is —CH₂O—, or a moiety represented by any one of the following structural formulas: 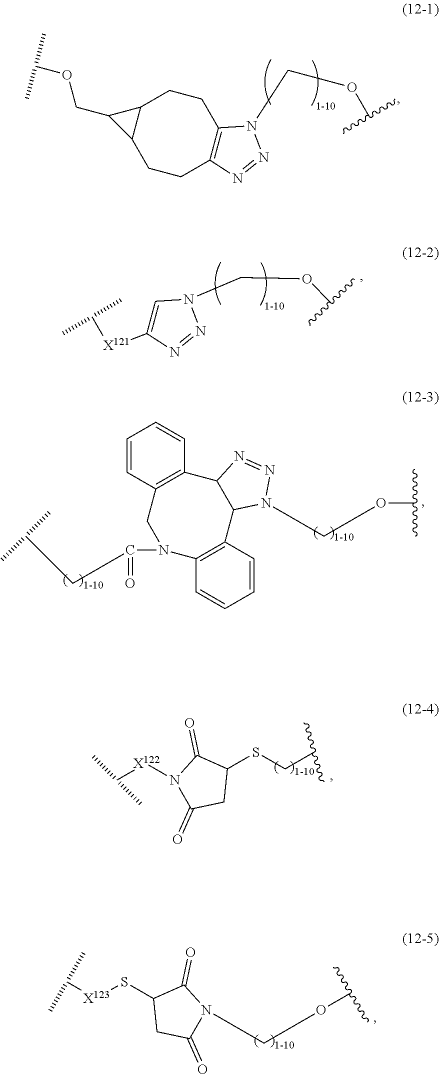
wherein X¹²¹, X¹², and X¹²³, each independently is —(CH₂)₁₋₁₀— or —CH₂CH₂—(OCH₂CH₂)₁₋₁₀, and wherein the symbol [symbol] represents the point of attachment to the carbonyl C; and 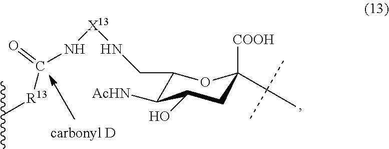
wherein:
X¹³ is -Ph- or —CH₂-Ph-CH₂—, wherein Ph is phenyl; and
R¹³ is is —CH₂O—, or a moiety represented by any one of the following structural formulas: 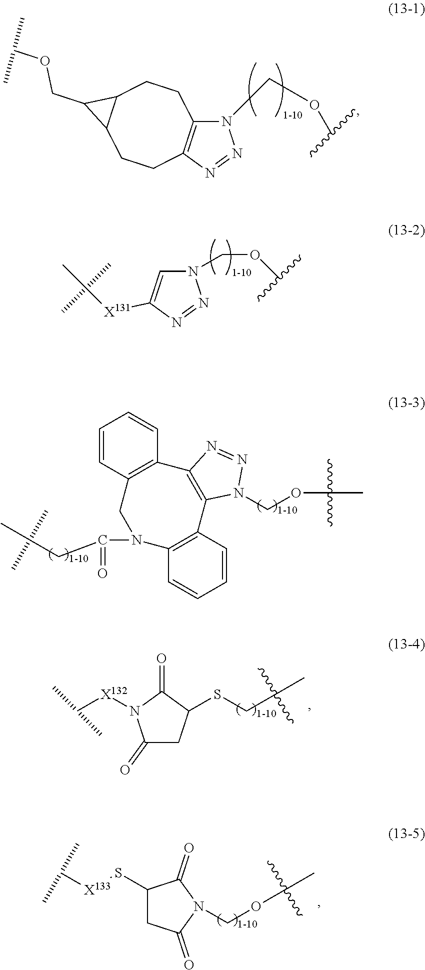
wherein X¹³¹, X¹³², and X¹³³, each independently is —(CH₂)₁₋₁₀— or —CH₂CH₂—(OCH₂CH₂)₁₋₁₀—, and wherein the symbol [symbol] represents the point of attachment to the carbonyl D; 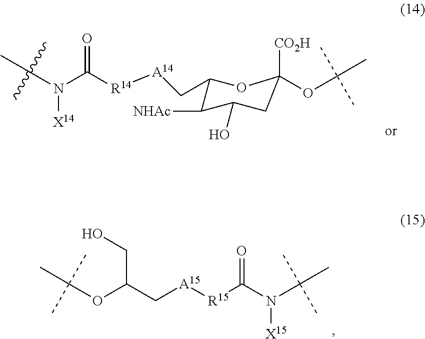
wherein:
X¹⁴ and X¹⁵ each independently is H or methyl, A¹⁴ and A¹⁵ each independently is NR^{A}, N^{A}N^{B}, O or S, wherein R^{A} and R^{B} for each occurrence independently is selected from H, C1-4 alkyl and C6-C18 aryl; and R¹⁴ and R¹⁵ each independently is —(CH₂)₁₋₁₀— or —CH₂CH₂—(OCH₂CH₂)₁₋₁₀—.

10. The particle of claim 9, wherein the linker L is represented by the following structural formula: 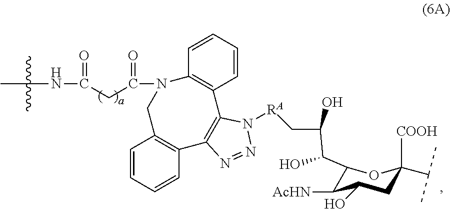
wherein:
a is an integer from 2 to 6; and
R^{A} is absent or is —(CH₂)₁₋₂—O—(CH₂)₁₋₂—NH—.

11. The particles of claim 1, wherein the P is a PLGA(10 k)-PEG(5 k).

12. The particle of claim 1, wherein the weight of G per unit weight of P (ligand density) is from 10 to 75 μg/mg.

13. A method of treating a subject suffering from an ophthalmic disease, comprising:
administering to the subject a therapeutically effective amount of claim 1.

14. The method of claim 13, wherein the ophthalmic disease is a dry age-related macular degeneration, a wet age-related macular degeneration, non-proliferative diabetic retinopathy, proliferative diabetic retinopathy, macular edema, uveitis, dry eyes, conjunctivitis, thyroid ophthalmopathy, endophthalmitis, retinal degeneration, glaucoma, retinal vein occlusions, blepharitis, keratitis, an ocular infection, or a cataract.

15-48. (canceled)

49. A pharmaceutical composition comprising a particle of claim 1, and a pharmaceutically acceptable carrier.

50. The pharmaceutical composition of claim 49, wherein the pharmaceutically acceptable carrier includes a PBS buffer or a saline solution.

51. The pharmaceutical composition of claim 49, wherein the concentration the particles in the carrier is from 0.01 mg/ml to 100 mg/ml.

52. A composition comprising the lyophilized or freeze-dried particle of claim 1.

53-63. (canceled)

64. The particle of claim 5, wherein P is PLGA(10 k)-PEG(5 k) and p is from 5 to 25.

65. The method of claim 13, wherein the ophthalmic disease is geographic atrophy.

66. The particle of claim 5, wherein p is from 20 to 30.

67. The method of claim 65, wherein the route of administration if intravitreal.

68. A particle, comprising a molecule represented by the following structural formula:
P-L-G,
wherein:
P is wherein P is PLGA(10 k)-PEG(5 k);
G is a polysialic acid (PSA) comprising from 20 to 30 repeat units of sialic acid; and
L is a covalent linker,
or a pharmaceutically acceptable salt thereof.

69. A method of treating geographic atrophy in a human patient, the method comprising intravitreally administering to the human patient the particle of claim 68.

70. A compound represented by the following structural formula: 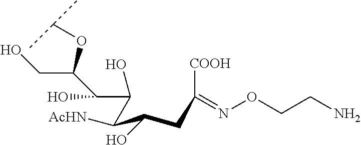
or a pharmaceutically acceptable salt thereof,
wherein symbol [symbol] a denotes a point of attachment to a polysialic acid.
